SEQUENCE LISTING (67 sequences)

SEQ ID NO: 1 - protein, 20 aa, Rattus norvegicus
  REGION 1..20: note = Dimer Blocker
MEPVPSARAELQFSLLANVS

SEQ ID NO: 2 - protein, 8 aa, Rattus norvegicus
  REGION 1..8: note = Dimer Blocker
SDTFPSAF

SEQ ID NO: 3 - protein, 9 aa, Rattus norvegicus
  REGION 1..9: note = Dimer Blocker
SDTFPSAFD

SEQ ID NO: 4 - protein, 11 aa, Rattus norvegicus
  REGION 1..11: note = Dimer Blocker
SDTFPSAFSSS

SEQ ID NO: 5 - protein, 18 aa, Rattus norvegicus
  REGION 1..18: note = Dimer Blocker
SDTFPSAFPSASANASGS

SEQ ID NO: 6 - protein, 19 aa, Rattus norvegicus
  REGION 1..19: note = Dimer Blocker
SDTFPSAFPSASANASGSD

SEQ ID NO: 7 - protein, 10 aa, Rattus norvegicus
  REGION 1..10: note = Dimer Blocker
NASGSPGARS

SEQ ID NO: 8 - protein, 23 aa, Rattus norvegicus
  REGION 1..23: note = Dimer Blocker
SDTFPSAFPSASANASGSPGARS

SEQ ID NO: 9 - protein, 23 aa, synthetic construct
  REGION 1..23: note = Dimer blocker
SPSSFAPGSNGATADFSPASASR

SEQ ID NO: 10 - protein, 31 aa, Rattus norvegicus
  REGION 1..31: note = Dimer Blocker
MDSSTGPGNTSDCSDPLAQASCSPAPGSWLN

SEQ ID NO: 11 - protein, 10 aa, Rattus norvegicus
  REGION 1..10: note = Dimer Blocker
LSHVDGNQSD

SEQ ID NO: 12 - protein, 10 aa, Rattus norvegicus
  REGION 1..10: note = Dimer Blocker
PCGLNRTGLG

SEQ ID NO: 13 - protein, 10 aa, Rattus norvegicus
  REGION 1..10: note = Dimer Blocker
GNDSLCPQTG

SEQ ID NO: 14 - protein, 9 aa, Rattus norvegicus
  REGION 1..9: note = Dimer Blocker
SPSMVTAIT

SEQ ID NO: 15 - protein, 30 aa, Rattus norvegicus
  REGION 1..30: note = Dimer Blocker
LSHVDGNQSDPCGLNRTGLGGNDSLCPQTG

SEQ ID NO: 16 - protein, 30 aa, synthetic construct
  REGION 1..30: note = Dimer Blocker
CVPRCQGSNGQHDGGTSGGNPDDNLLSLTL

SEQ ID NO: 17 - protein, 10 aa, Rattus norvegicus
  REGION 1..10: note = Dimer Blocker
RRDPLVVAAL

SEQ ID NO: 18 - protein, 17 aa, Rattus norvegicus
  REGION 1..17: note = Dimer Blocker
WTLVDINRRDPLVVAAL

SEQ ID NO: 19 - protein, 16 aa, Rattus norvegicus
  REGION 1..16: note = Dimer Blocker
EALGSTSHSTAVLSSY

SEQ ID NO: 20 - protein, 8 aa, Homo sapiens
  REGION 1..8: note = Dimer Blocker
SDAYPSAC

SEQ ID NO: 21 - protein, 18 aa, Homo sapiens
  REGION 1..18: note = Dimer Blocker
SDAYPSACPSAGANASGP

SEQ ID NO: 22 - protein, 10 aa, Homo sapiens
  REGION 1..10: note = Dimer Blocker
NASGPPGARS

SEQ ID NO: 23 - protein, 23 aa, Homo sapiens
  REGION 1..23: note = Dimer Blocker
SDAYPSACPSAGANASGPPGARS

SEQ ID NO: 24 - protein, 10 aa, Homo sapiens
  REGION 1..10: note = Dimer Blocker
LSHLDGNLSD

SEQ ID NO: 25 - protein, 10 aa, Homo sapiens
  REGION 1..10: note = Dimer Blocker
PCGPNRTDLG

SEQ ID NO: 26 - protein, 10 aa, Homo sapiens
  REGION 1..10: note = Dimer Blocker
GRDSLCPPTG

SEQ ID NO: 27 - protein, 30 aa, Homo sapiens
  REGION 1..30: note = Dimer Blocker
LSHLDGNLSDPCGPNRTDLGGRDSLCPPTG

SEQ ID NO: 28 - protein, 10 aa, Homo sapiens
  REGION 1..10: note = Dimer Blocker
RRDPLVVAAL

SEQ ID NO: 29 - protein, 17 aa, Homo sapiens
  REGION 1..17: note = Dimer Blocker
WTLVDIDRRDPLVVAAL

SEQ ID NO: 30 - protein, 16 aa, Homo sapiens
  REGION 1..16: note = Dimer Blocker
EALGSTSHSTAALSSY

SEQ ID NO: 31 - protein, 45 aa, Rattus norvegicus
  REGION 1..45: note = Dimer Blocker
EQQNSTRVRQNTREHPSTANTVDRGGGGGGGGGGYGRKKRRQRRR

SEQ ID NO: 32 - protein, 37 aa, Rattus norvegicus
  REGION 1..37: note = Dimer Blocker
QDPASMRDVGGMNKPVGGGGGGGGGGYGRKKRRQRRR

SEQ ID NO: 33 - protein, 30 aa, Rattus norvegicus
  REGION 1..30: note = Dimer Blocker
DVGGMNKPVGGGGGGGGGGYGRKKRRQRRR

SEQ ID NO: 34 - protein, 37 aa, Rattus norvegicus
  REGION 1..37: note = Dimer Blocker
VTACTPSDGPGGGAAAGGGGGGGGGGYGRKKRRQRRR

SEQ ID NO: 35 - protein, 55 aa, Rattus norvegicus
  REGION 1..55: note = Dimer Blocker
GQEPGSLRRPRQATARERVTACTPSDGPGGGAAAGGGGGGGGGGYGRKKRRQRRR

SEQ ID NO: 36 - protein, 46 aa, Homo sapiens
  REGION 1..46: note = Dimer Blocker
IEQQNSTRIRQNTRDHPSTANTVDRGGGGGGGGGGYGRKKRRQRRR

SEQ ID NO: 37 - protein, 38 aa, Homo sapiens
  REGION 1..38: note = Dimer Blocker
VQDPAYLRDIDGMNKPVGGGGGGGGGGYGRKKRRQRRR

SEQ ID NO: 38 - protein, 30 aa, Homo sapiens
  REGION 1..30: note = Dimer Blocker
DIDGMNKPVGGGGGGGGGGYGRKKRRQRRR

SEQ ID NO: 39 - protein, 37 aa, Homo sapiens; identical to SEQ ID NO: 34
  REGION 1..37: note = Dimer Blocker

SEQ ID NO: 40 - protein, 55 aa, Homo sapiens
  REGION 1..55: note = Dimer Blocker
RPDPSSFSRAREATARERVTACTPSDGPGGGAAAGGGGGGGGGGYGRKKRRQRRR

SEQ ID NO: 41 - protein, 23 aa, Rattus norvegicus
  REGION 1..23: note = Dimer Blocker
  SITE 1: note = D-Serine
  SITE 2: note = D-Aspartic Acid
  SITE 3: note = D-Threonine
  SITE 21: note = D-Alanine
  SITE 22: note = D-Arginine
  SITE 23: note = D-Serine
SDTFPSAFPSASANASGSPGARS

SEQ ID NO: 42 - protein, 44 aa, Rattus norvegicus
  REGION 1..44: note = Dimer Blocker
  SITE 1: note = D-Serine
  SITE 2: note = D-Aspartic Acid
  SITE 3: note = D-Threonine
  SITE 42..44: note = D-Arginine
SDTFPSAFPSASANASGSPGARSGGGGGGGGGGYGRKKRRQRRR

SEQ ID NO: 43 - protein, 8 aa, Homo sapiens
  REGION 1..8: note = Dimer Blocker
  SITE 1: note = D-Serine
  SITE 2: note = D-Aspartic Acid
  SITE 3: note = D-Alanine
  SITE 6: note = D-Serine
  SITE 7: note = D-Alanine
  SITE 8: note = D-Cysteine
SDAYPSAC

SEQ ID NO: 44 - protein, 29 aa, Homo sapiens
  REGION 1..29: note = Dimer Blocker
  SITE 1: note = D-Serine
  SITE 2: note = D-Aspartic Acid
  SITE 3: note = D-Alanine
  SITE 27..29: note = D-Arginine
SDAYPSACGGGGGGGGGGYGRKKRRQRRR

SEQ ID NO: 45 - protein, 18 aa, Homo sapiens
  REGION 1..18: note = Dimer Blocker
  SITE 1: note = D-Serine
  SITE 2: note = D-Aspartic Acid
  SITE 3: note = D-Alanine
  SITE 16: note = D-Serine
  SITE 17: note = D-Glycine
  SITE 18: note = D-Proline
SDAYPSACPSAGANASGP

SEQ ID NO: 46 - protein, 39 aa, Homo sapiens
  REGION 1..39: note = Dimer Blocker
  SITE 1: note = D-Serine
  SITE 2: note = D-Aspartic Acid
  SITE 3: note = D-Alanine
  SITE 37..39: note = D-Arginine
SDAYPSACPSAGANASGPGGGGGGGGGGYGRKKRRQRRR

SEQ ID NO: 47 - protein, 23 aa, Homo sapiens
  REGION 1..23: note = Dimer Blocker
  SITE 1: note = D-Serine
  SITE 2: note = D-Aspartic Acid
  SITE 3: note = D-Alanine
  SITE 21: note = D-Alanine
  SITE 22: note = D-Arginine
  SITE 23: note = D-Serine
SDAYPSACPSAGANASGPPGARS

SEQ ID NO: 48 - protein, 44 aa, Homo sapiens
  REGION 1..44: note = Dimer Blocker
  SITE 1: note = D-Serine
  SITE 2: note = D-Aspartic Acid
  SITE 3: note = D-Alanine
  SITE 42..44: note = D-Arginine
SDAYPSACPSAGANASGPPGARSGGGGGGGGGGYGRKKRRQRRR

SEQ ID NO: 49 - protein, 10 aa, Homo sapiens
  REGION 1..10: note = Dimer Blocker
  SITE 1: note = D-Leucine
  SITE 2: note = D-Serine
  SITE 3: note = D-Histidine
  SITE 8: note = D-Leucine
  SITE 9: note = D-Serine
  SITE 10: note = D-Aspartic Acid
LSHLDGNLSD

SEQ ID NO: 50 - protein, 31 aa, Homo sapiens
  REGION 1..31: note = Dimer Blocker
  SITE 1: note = D-Leucine
  SITE 2: note = D-Serine
  SITE 3: note = D-Histidine
  SITE 29..31: note = D-Arginine
LSHLDGNLSDGGGGGGGGGGYGRKKRRQRRR

SEQ ID NO: 51 - protein, 10 aa, Homo sapiens
  REGION 1..10: note = Dimer Blocker
  SITE 1: note = D-Proline
  SITE 2: note = D-Cysteine
  SITE 3: note = D-Glycine
  SITE 8: note = D-Aspartic Acid
  SITE 9: note = D-Leucine
  SITE 10: note = D-Glycine
PCGPNRTDLG

SEQ ID NO: 52 - protein, 31 aa, Homo sapiens
  REGION 1..31: note = Dimer Blocker
  SITE 1: note = D-Proline
  SITE 2: note = D-Cysteine
  SITE 3: note = D-Glycine
  SITE 29..31: note = D-Arginine
PCGPNRTDLGGGGGGGGGGGYGRKKRRQRRR

SEQ ID NO: 53 - protein, 10 aa, Homo sapiens
  REGION 1..10: note = Dimer Blocker
  SITE 1: note = D-Glycine
  SITE 2: note = D-Arginine
  SITE 3: note = D-Aspartic Acid
  SITE 8: note = D-Proline
  SITE 9: note = D-Threonine
  SITE 10: note = D-Glycine
GRDSLCPPTG

SEQ ID NO: 54 - protein, 31 aa, Homo sapiens
  REGION 1..31: note = Dimer Blocker
  SITE 1: note = D-Glycine
  SITE 2: note = D-Arginine
  SITE 3: note = D-Aspartic Acid
  SITE 29..31: note = D-Arginine
GRDSLCPPTGGGGGGGGGGGYGRKKRRQRRR

SEQ ID NO: 55 - protein, 30 aa, Homo sapiens; identical to SEQ ID NO: 27
  REGION 1..30: note = Dimer Blocker
  SITE 1: note = D-Leucine
  SITE 2: note = D-Serine
  SITE 3: note = D-Histidine
  SITE 28: note = D-Proline
  SITE 29: note = D-Threonine
  SITE 30: note = D-Glycine

SEQ ID NO: 56 - protein, 51 aa, Homo sapiens
  REGION 1..51: note = Dimer Blocker
  SITE 1: note = D-Leucine
  SITE 2: note = D-Serine
  SITE 3: note = D-Histidine
  SITE 49..51: note = D-Arginine
LSHLDGNLSDPCGPNRTDLGGRDSLCPPTGGGGGGGGGGGYGRKKRRQRRR

SEQ ID NO: 57 - protein, 10 aa, Homo sapiens
  REGION 1..10: note = Dimer Blocker
  SITE 1..2: note = D-Arginine
  SITE 3: note = D-Aspartic Acid
  SITE 8..9: note = D-Alanine
  SITE 10: note = D-Leucine
RRDPLVVAAL

SEQ ID NO: 58 - protein, 31 aa, Homo sapiens
  REGION 1..31: note = Dimer Blocker
  SITE 1..2: note = D-Arginine
  SITE 3: note = D-Aspartic Acid
  SITE 29..31: note = D-Arginine
RRDPLVVAALGGGGGGGGGGYGRKKRRQRRR

SEQ ID NO: 59 - protein, 17 aa, Homo sapiens
  REGION 1..17: note = Dimer Blocker
  SITE 1: note = D-Tryptophan
  SITE 2: note = D-Threonine
  SITE 3: note = D-Leucine
  SITE 15..16: note = D-Alanine
  SITE 17: note = D-Leucine
WTLVDIDRRDPLVVAAL

SEQ ID NO: 60 - protein, 38 aa, Homo sapiens
  REGION 1..38: note = Dimer Blocker
  SITE 1: note = D-Tryptophan
  SITE 2: note = D-Threonine
  SITE 3: note = D-Leucine
  SITE 36..38: note = D-Arginine
WTLVDIDRRDPLVVAALGGGGGGGGGGYGRKKRRQRRR

SEQ ID NO: 61 - protein, 16 aa, Homo sapiens
  REGION 1..16: note = Dimer Blocker
  SITE 1: note = D-Glutamic Acid
  SITE 2: note = D-Alanine
  SITE 3: note = D-Leucine
  SITE 14..15: note = D-Serine
  SITE 16: note = D-Tyrosine
EALGSTSHSTAALSSY

SEQ ID NO: 62 - protein, 37 aa, Homo sapiens
  REGION 1..37: note = Dimer Blocker
  SITE 1: note = D-Glutamic Acid
  SITE 2: note = D-Alanine
  SITE 3: note = D-Leucine
  SITE 35..37: note = D-Arginine
EALGSTSHSTAALSSYGGGGGGGGGGYGRKKRRQRRR

SEQ ID NO: 63 - protein, 46 aa, Homo sapiens; identical to SEQ ID NO: 36
  REGION 1..46: note = Dimer Blocker
  SITE 1: note = D-Isoleucine
  SITE 2: note = D-Glutamic Acid
  SITE 3: note = D-Glutamine
  SITE 44..46: note = D-Arginine

SEQ ID NO: 64 - protein, 38 aa, Homo sapiens; identical to SEQ ID NO: 37
  REGION 1..38: note = Dimer Blocker
  SITE 1: note = D-Valine
  SITE 2: note = D-Glutamine
  SITE 3: note = D-Aspartic Acid
  SITE 36..38: note = D-Arginine

SEQ ID NO: 65 - protein, 30 aa, Homo sapiens; identical to SEQ ID NO: 38
  REGION 1..30: note = Dimer Blocker
  SITE 1: note = D-Aspartic Acid
  SITE 2: note = D-Isoleucine
  SITE 3: note = D-Aspartic Acid
  SITE 28..30: note = D-Arginine

SEQ ID NO: 66 - protein, 37 aa, Homo sapiens; identical to SEQ ID NO: 34
  REGION 1..37: note = Dimer Blocker
  SITE 1: note = D-Valine
  SITE 2: note = D-Threonine
  SITE 3: note = D-Alanine
  SITE 35..37: note = D-Arginine

SEQ ID NO: 67 - protein, 55 aa, Homo sapiens; identical to SEQ ID NO: 40
  REGION 1..55: note = Dimer Blocker
  SITE 1: note = D-Arginine
  SITE 2: note = D-Proline
  SITE 3: note = D-Aspartic Acid
  SITE 53..55: note = D-Arginine

(19) United States
(12) Patent Application Publication
(10) Pub. No.: US 2025/0368686 A1
(43) Pub. Date: Dec. 4, 2025

(54) PEPTIDE DRUGS FOR SUPPRESSING TOLERANCE DEVELOPMENT BY BLOCKING HOMO- AND HETERO-DIMERIZATION OF OPIOID RECEPTORS

(71) Applicant: OKINAWA INSTITUTE OF SCIENCE AND TECHNOLOGY SCHOOL CORPORATION, Okinawa (JP)

(72) Inventors: Akihiro KUSUMI, Okinawa (JP); Peng ZHOU, Okinawa (JP)

(73) Assignee: OKINAWA INSTITUTE OF SCIENCE AND TECHNOLOGY SCHOOL CORPORATION, Okinawa (JP)

(21) Appl. No.: 18/874,361

(22) PCT Filed: Jun. 15, 2023
(86) PCT No.: PCT/JP2023/022333

§ 371 Date: Dec. 12, 2024

(30) Foreign Application Priority Data
Jun. 15, 2022 (JP) 2022-096238

(51) Int. Cl. C07K 14/00; A61K 38/00; A61P 25/00

(52) U.S. Cl. CPC C07K 14/001; A61P 25/00; A61K 38/00

(57) ABSTRACT

Problem The purpose of the present invention is to provide a novel method for prevention and/or treatment of opioid tolerance or opioid dependence. Specifically, the purpose of the present invention is to develop peptide-based blockers to block MOR-DOR and KOR-DOR heterodimerization. In addition, the purpose of the present invention is to develop homodimer blockers for MOR, DOR, and KOR which modulate their downstream signaling without affecting their internalization. Solution The present invention is a prophylactic and/or therapeutic agent for the prevention and/or treatment of opioid tolerance or opioid dependence comprising the peptide which inhibits the dimer formation of the opioid receptor, wherein the above opioid receptor dimer is a heterodimer or a homodimer formed from one or two opioid receptors selected from the group consisting of the μ type (MOR), the κ type (KOR), and the δ type (DOR).

### TECHNICAL FIELD

This patent application claims priority to Japanese Patent Application No. 2022-096238, filed on Jun. 15, 2022, which is incorporated herein by reference in its entirety.

The present invention relates to the peptide drugs for suppressing tolerance development by blocking homo- and hetero-dimerization of opioid receptors.

### BACKGROUND ART

The opioids are important for clinical pain management, but repeated use reduces their analgesic effect, called “tolerance”, and thus, long-term opioid therapy is associated with increased risk of abuse, fatal overdose, dependence, and addiction, leading to serious clinical and social problems. Therefore, methods to suppress tolerance development to opioids are badly needed.

Three classical opioid receptors (OPRs), μ-, κ-, and δ-OPRs (MOR, KOR, and DOR), distribute throughout the central and peripheral nervous system and play important roles in regulating pain perception, hedonic homeostasis, mood, and well-being (NPL 1, 2). OPRs are prototypical class A G-protein-coupled receptors (GPCRs), and as the key receptors for a variety of endogenous and synthetic analgesics, these classical OPRs are among one of the most important druggable GPCRs. These OPR subtypes play critical roles in the development of tolerance and dependence by signaling through inhibitory G proteins and arrestin, leading to different levels of desirable and adverse drug responses (NPL 3, 4).

One approach to solve the problems such as opioid misuse/abuse, tolerance, addiction, and dependence is to increase treatment diversity (Schmid et al., cell 2017; NPL 23), including the regulation of OPR heterodimerisation (Ong and Cahill, 2014; NPL 27; Gaborit and Massotte B J P 2021; NPL 28); however, the characteristics of OPR heterodimers remain enigmatic.

DOR and MOR (DM) heterodimers (George et al., JBC 2000; NPL 29; Gomes and Devi, PNAS 2004; NPL 30; Xic and Wang, J N 2009; NPL 31; Chefer and Shippenberg, Neuropsychopharm 2009; NPL 32; Wang et al., Neuron 2018; NPL 33) and DOR and KOR (DK) heterodimers (Jordan and Devi, Nature 1999; NPL 34; Waldhoer et al., PNAS 2005; NPL 35; Ansonoff et al., Psychopharmacology 2010; NPL 39; Jacobs et al., Mol Pharm. 2018; NPL 37; Jacobs et al., Neuropharm. 2019; NPL 38) have been found both in vitro and in vivo, whereas MOR and KOR (MK) heterodimers have not been detected (Jordan and Devi, Nature 1999; NPL 34). DOR and MOR are co-expressed in dorsal horn projection neurons and the ventral horn (Wang et al., Neuron 2018; NPL 33). The DOR-KOR heteromers were detected by coimmunoprecipitation assays in rat pain-sensing neurons (Berg et al., Mol. Pharm. 2012; NPL 36).

The pharmacological importance of DM and DK heterodimers has been demonstrated. In the mouse central nervous system, chronic morphine treatment increased DM heteromers in pain processing (Gupta et al., 2010; NPL 40). In addition, the suppression of DM heteromer formation by delivering the first transmembrane domain of MOR (MOR-TM1) reduced the antinociceptive tolerance to morphine, probably due to the blockage of DM co-degradation (He et al., Neuron 2011; NPL 41). The MOR-mediated spinal analgesia is negatively regulated by DOR activation, and the tolerance to opioids is reduced by the pharmacological blocking or genetic deletion of DOR (Gomes and Devi, PNAS 2004; NPL 30; Xic and Wang, J N 2009; NPL 31; Chefer and Shippenberg, Neuropsychopharm 2009; NPL 32). Notably, the DOR and KOR signals were affected by the DOR-KOR heterodimer interactions (Berg et al., Mol. Pharm. 2012; NPL 36; Jacobs et al., Mol Pharm. 2018; NPL 37; Jacobs et al., Neuropharm. 2019; NPL 38).

Despite these extensive studies, the fundamental characteristics of DM and DK heterodimers, including their lifetimes, sites responsible for heterodimerisation, and effects on signaling and internalisation, have remained enigmatic.

Another approach to solve the problems such as opioid misuse/abuse, tolerance, addiction, and dependence is to increase treatment diversity, including the regulation of OPR homodimerisation (NPL 5-8).

The monomeric OPRs may be able to simply activate signaling cascades, but for example, in the case of MOR, the changes of monomer-homodimer equilibrium induced by different agonists were proposed to play important roles in regulating the relative strengths of downstream signals (NPL 9).

Meanwhile, the homodimerization of OPRs (and class-A GPCRs) remains controversial, and the functions of homodimers are mostly unknown. The first report about the OPR homodimerization was made by Cvejic and Devi in 1997 for DOR (NPL 10), which employed a biochemical assay. A number of reports using biochemical assays followed that indicate the homodimerization of DOR (NPL 11), MOR (NPL 7,11,13,14) and KOR (NPL 7,15). Furthermore, the optical measurements of the cells overexpressing OPRs found homodimerization (NPL 7, 12, 16, 17), except for one report (NPL 18).

These conclusions were challenged by five single-molecule imaging studies of OPRs expressed at lower physiological concentrations in the plasma membrane (PM) of living cells. They found that MOR (NPL 9, 19, 20, 21), DOR (NPL 21) and KOR (NPL 22) are monomeric or homodimer affinities are quite low. For example, KOR is monomeric at densities <10 copies/μm² and the dimers are detectable at densities >25 copies/μm² (the authors claimed that this expression level is within the physiological range) (NPL 21). These studies cast strong doubts about the interpretation of the previous OPR homodimerization data obtained by biochemical methods in vitro or under overexpression conditions in the cellular PM.

### CITATION LIST

### Non Patent Literature

NPL 1: Darcq, E. & Kieffer, B. L. Opioid receptors: drivers to addiction? Nat. Rev. Neurosci. 19, 499-514 (2018).
NPL 2: Valentino, R. J. & Volkow, N. D. Untangling the complexity of opioid receptor function. Neuropsychopharmacology 43, 2514-2520 (2018).
NPL 3: Schmid, C. L. et al. Bias factor and therapeutic window correlate to predict safer opioid analgesics. Cell 171, 1165-1175.e1113 (2017).
NPL 4: Sungkaworn, T. et al. Single-molecule imaging reveals receptor-G protein interactions at cell surface hot spots. Nature 550, 543-547 (2017).
NPL 5: Rios, C. D., Jordan, B. A., Gomes, I. & Devi, L. A. G-protein-coupled receptor dimerization: modulation of receptor function. Pharmacol. Ther. 92, 71-87 (2001).
NPL 6: Terrillon, S. & Bouvier, M. Roles of G-protein-coupled receptor dimerization. EMBO reports 5, 30-34 (2004).
NPL 7: Ferre, S. et al. G protein-coupled receptor oligomerization revisited: functional and pharmacological perspectives. Pharmacol. Rev. 66, 413 (2014).
NPL 8: Milligan, G., Ward, R. J. & Marsango, S. GPCR homo-oligomerization. Curr. Opin. Cell Biol. 57, 40-47 (2019).
NPL 9: Moller, J. et al. Single-molecule analysis reveals agonist-specific dimer formation of μ-opioid receptors. Nat. Chem. Biol. 16, 946-954 (2020).
NPL 10: Cvejic, S. & Devi, L. A. Dimerization of the delta opioid receptor: implication for a role in receptor internalization. J. Biol. Chem. 272, 26959-26964 (1997).
NPL 11: George, S. R. et al. Oligomerization of mu- and delta-opioid receptors: Generation of novel functional properties. J. Biol. Chem. 275, 26128-26135 (2000).
NPL 12: McVey, M. et al. Monitoring receptor oligomerization using time-resolved fluorescence resonance energy transfer and bioluminescence resonance energy transfer: The human δ-opioid receptor displays constitutive oligomerization at the cell surface, which is not regulated by receptor occupancy. J. Biol. Chem. 276, 14092-14099 (2001).
NPL 13: He, L., Fong, J., von Zastrow, M. & Whistler, J. L. Regulation of opioid receptor trafficking and morphine tolerance by receptor oligomerization. Cell 108, 271-282 (2002).
NPL 14: Zheng, H. et al. Palmitoylation and membrane cholesterol stabilize μ-opioid receptor homodimerization and G protein coupling. BMC Cell Biol. 13, 6 (2012).
NPL 15: Jordan, B. A. & Devi, L. A. G-protein-coupled receptor heterodimerization modulates receptor function. Nature 399, 697-700 (1999).
NPL 16: Gomes, I., Filipovska, J., Jordan, B. A. & Devi, L. A. Oligomerization of opioid receptors. Methods 27, 358-365 (2002).
NPL 17: Wang, D., Sun, X., Bohn, L. M. & Sadee, W. Opioid receptor homo- and heterodimerization in living cells by quantitative bioluminescence resonance energy transfer. Mol. Pharmacol. 67, 2173-2184 (2005).
NPL 18: Meral, D. et al. Molecular details of dimerization kinetics reveal negligible populations of transient μ-opioid receptor homodimers at physiological concentrations. Sci. Rep. 8, 7705 (2018).
NPL 19: Gentzsch, C. et al. Selective and wash-resistant fluorescent dihydrocodeinone derivatives allow single-molecule imaging of μ-opioid receptor dimerization. Angew. Chem. Int. Ed. 59, 5958-5964 (2020).
NPL 20: Asher, W. B. et al. Single-molecule fret imaging of GPCR dimers in living cells. Nat. Methods 18, 397-405 (2021).
NPL 21: Cechova, K. et al. Kappa but not delta or mu opioid receptors form homodimers at low membrane densities. Cell. Mol. Life Sci. 78, 7557-7568 (2021).
NPL 22: Drakopoulos, A. et al. Investigation of inactive-state κ opioid receptor homodimerization via single-molecule microscopy using new antagonistic fluorescent probes. J. Med. Chem. 63, 3596-3609 (2020).
NPL 23: Schmid, C. L. et al. Bias factor and therapeutic window correlate to predict safer opioid analgesics. Cell 171, 1165-1175.e1113, doi:10.1016/j.cell.2017.10.035 (2017).
NPL 24: Sungkaworn, T. et al. Single-molecule imaging reveals receptor-G protein interactions at cell surface hot spots. Nature 550, 543-547, doi:10.1038/nature24264 (2017).
NPL 25: Darcq, E. & Kieffer, B. L. Opioid receptors: drivers to addiction? Nat. Rev. Neurosci. 19, 499-514, doi:10.1038/s41583-018-0028-x (2018).
NPL 26: Valentino, R. J. & Volkow, N. D. Untangling the complexity of opioid receptor function. Neuropsychopharmacology 43, 2514-2520, doi:10.1038/s41386-018-0225-3 (2018).
NPL 27: Ong, E. W. & Cahill, C. M. Molecular Perspectives for mu/delta Opioid Receptor Heteromers as Distinct, Functional Receptors. Cells 3, 152-179 (2014).
NPL 28: Gaborit, M. & Massotte, D. Therapeutic potential of opioid receptor heteromers in chronic pain and associated comorbidities. British Journal of Pharmacology n/a, doi:https://doi.org/10.1111/bph.15772.
NPL 29: George, S. R. et al. Oligomerization of mu- and delta-opioid receptors.
Generation of novel functional properties. J. Biol. Chem. 275, 26128-26135, doi:10.1074/jbc.M000345200 (2000).
NPL 30: Gomes, I. et al. A role for heterodimerization of μ and δ opiate receptors in enhancing morphine analgesia. Proc. Natl. Acad. Sci. U.S.A. 101, 5135, doi:10.1073/pnas.0307601101 (2004).
NPL 31: Xie, W.-Y. et al. Disruption of Cdk5-associated phosphorylation of residue threonine-161 of the 8-Opioid Receptor: impaired receptor function and attenuated morphine antinociceptive tolerance. J. Neurosci. 29, 3551, doi:10.1523/JNEUROSCI.0415-09.2009 (2009).
NPL 32: Chefer, V. I. & Shippenberg, T. S. Augmentation of morphine-induced sensitization but reduction in morphine tolerance and reward in delta-opioid receptor knockout mice. Neuropsychopharmacology 34, 887-898, doi:10.1038/npp.2008.128 (2009).
NPL 33: Wang, D. et al. Functional divergence of delta and mu opioid receptor organization in CNS Pain Circuits. Neuron 98, 90-108.c105, doi:https://doi.org/10.1016/j.neuron.2018.03.002 (2018).
NPL 34: Jordan, B. A. & Devi, L. A. G-protein-coupled receptor heterodimerization modulates receptor function. Nature 399, 697-700, doi:10.1038/21441 (1999).
NPL 35: Waldhoer, M. et al. A heterodimer-selective agonist shows in vivo relevance of G protein-coupled receptor dimers. Proc. Natl. Acad. Sci. U.S.A. 102, 9050, doi:10.1073/pnas.0501112102 (2005).
NPL 36: Berg, K. A. et al. Allosteric interactions between δ and κ opioid receptors in peripheral sensory neurons. Mol. Pharmacol. 81, 264, doi:10.1124/mol.111.072702 (2012).
NPL 37: Jacobs, B. A. et al. Allosterism within δ opioid-κ Opioid Receptor heteromers in peripheral sensory neurons: regulation of κ opioid agonist efficacy. Mol. Pharmacol. 93, 376-386, doi:10.1124/mol.117.109975 (2018).
NPL 38: Jacobs, B. A. et al. Signaling characteristics and functional regulation of delta opioid-kappa opioid receptor (DOP-KOP) heteromers in peripheral sensory neurons. Neuropharmacology 151, 208-218, doi:https://doi.org/10.1016/j.neuropharm.2019.02.019 (2019).
NPL 39: Ansonoff, M. A., Portoghese, P. S. & Pintar, J. E. Consequences of opioid receptor mutation on actions of univalent and bivalent kappa and delta ligands. Psychopharmacology 210, 161-168, doi:10.1007/s00213-010-1826-7 (2010).
NPL 40: Gupta, A. et al. Increased abundance of opioid receptor heteromers after chronic morphine administration. Sci. Signal. 3, ra54-ra54, doi:10.1126/scisignal.2000807 (2010).
NPL 41: He, S.-Q. et al. Facilitation of μ-opioid receptor activity by preventing δ-opioid receptor-mediated codegradation. Neuron 69, 120-131, doi:https://doi.org/10.1016/j.neuron.2010.12.001 (2011).

### SUMMARY OF INVENTION

### Technical Problem

The opioids are important for clinical pain management, but repeated use can produce physiological tolerance and dependence. The clinical manifestations are mainly that the analgesic effect is reduced, and the analgesic effect is gradually weakened or even disappears after the opioid is continuously given, and the same analgesic effect can be obtained only by increasing the dosage of the opioid. Meanwhile, long-term opioid therapy is associated with increased risk of abuse, dependence, and dose-related fatal overdose.

The first purpose of the present invention is to suppress tolerance development to morphine. Currently, morphine stands out among all analgesics and its receptor MOR is most important in opioid-induced analgesia and reward processing. Meanwhile, it is well known that the MOR-mediated analgesia is negatively regulated by the heterodimerization with DOR, followed by co-internalization of MOR and DOR. Therefore, the first purpose of the present invention is to develop peptide-based blockers to block MOR-DOR heterodimerization.

The second purpose of the present invention is to develop blockers for KOR-DOR heterodimerization. It has been known that KOR and DOR form heterodimers although how it could affect the analgesic effects of various chemicals binding to KOR and DOR is unknown. Considering the strong effect of MOR-DOR heterodimer blockers on the MOR and DOR signals and internalization, KOR-DOR heterodimer blockers are potentially quite useful.

The third purpose of the present invention is to develop homodimer blockers for MOR, DOR, and KOR, which modulate their downstream signaling without affecting their internalization.

### Solution to Problem

The inventors invented peptide drugs for enhancing analgesia and suppressing tolerance development to morphine, the gold standard of opioid-based analgesia, and possibly to other analgesics. These peptides have not been known so far. They work by modulating the opioid receptor functions. One of the peptides was found to suppress the tolerance development to morphine in mouse. The opioids work by binding to opioid receptors (OPRs) located on the surface of neurons in the neuronal circuits that regulate pain perception, hedonic homeostasis, mood, and well-being. Three classical OPRs, called μ-, κ-, and δ-OPRs (MOR, KOR, and DOR, respectively) exist in our body.

The inventors found homo- and hetero-dimerization of OPRs, the methods to block their dimerization using peptide drugs, and that such inhibitions modulate the OPRs' downstream signals and OPR internalization, leading to this invention.

The present invention includes the following embodiments:
(1) A prophylactic and/or therapeutic agent for the prevention and/or treatment of opioid tolerance or opioid dependence comprising a peptide which inhibits the formation of a dimer, wherein the dimer is a heterodimer or a homodimer formed from one or two opioid receptors selected from the group consisting of the μ type (MOR), the κ type (KOR) and the δ type (DOR).
(2) The prophylactic and/or therapeutic agent for the prevention and/or treatment of opioid tolerance or opioid dependence according to (1), wherein the peptide comprises any one amino acid sequence selected from the group consisting of the amino acid sequences of Sequence ID Numbers 1 to 40, amino acid sequences having one or more conservative amino acid substitutions in the amino acid sequences of Sequence ID Numbers 1 to 40, the amino acid sequences of Sequence ID Numbers 41 to 67 listed in Table B, amino acid sequences having one or more conservative amino acid substitutions in the amino acid sequences of Sequence ID Numbers 41 to 67 listed in Table B and amino acid sequences having at least 80% amino acid sequence identity with any of these amino acid sequences.

TABLE 1
Table B. Dimer blockers (comprising
D-type-amino acid)

| SQ | | | |
|---|---|---|---|
| ID | | | Amino acid |
| No. | No. | | sequence |
| 41 | D1 | DpepDM | S[[D]D[D]T[D] |
| | | (20-42) | FPSAFPSASANA |
| | | (D-type | SGSPGA |
| | | aa at | [D]R[D]S[D] |
| | | N/C- | |
| | | termini) | |
| 42 | D2 | DpepDM | S[D]D[D]T[D] |
| | | (20-42)- | FPSAFPSASANA |
| | | TAT | SGSPGARSGGGGG |
| | | (D-type | GGGGGYGRKKRRQR |
| | | aa at | [D]R[D]R[D] |
| | | N/C- | |
| | | termini) | |
| 43 | D3 | DpepDM | S[D]D[D]A[D] |
| | | (20-27) | YPS |
| | | (D-type | [D]A[D]C[D] |
| | | aa at | |
| | | N/C- | |
| | | termini)- | |
| | | Human | |
| 44 | D4 | DpepDM | S[D]D[D]A[D] |
| | | (20-27)- | YPSACGGGGGGG |
| | | TAT | GGGYGRKKRRQR |
| | | (D-type | [D]R[D]R[D] |
| | | aa at | |
| | | N/C- | |
| | | termini)- | |
| | | Human | |
| 45 | D5 | DpepDM | S[D]D[D]A[D] |
| | | (20-37) | YPSACPSAGANAS |
| | | (D-type | [D]G[D]P[D] |
| | | aa at | |
| | | N/C- | |
| | | termini)- | |
| | | Human | |
| 46 | D6 | DpepDM | S[D]D[D]A[D] |
| | | (20-37)- | YPSACPSAGANA |
| | | TAT | SGPGGGGGGGGG |
| | | (D-type | GYGRKKRRQR |
| | | aa at | [D]R[D]R[D] |
| | | N/C- | |
| | | termini)- | |
| | | Human | |
| 47 | D7 | DpepDM | S[D]D[D]A[D] |
| | | (20-42) | YPSACPSAGANA |
| | | (D-type | SGPPGA |
| | | aa at | [D]R[D]S[D] |
| | | N/C- | |
| | | termini)- | |
| | | Human | |
| 48 | D8 | DpepDM | S[D]D[D]A[D] |
| | | (20-42)- | YPSACPSAGANA |
| | | TAT | SGPPGARSGGGGGG |
| | | (D-type | GGGGYGRKKRRQR |
| | | aa at | [D]R[D]R[D] |
| | | N/C- | |
| | | termini)- | |
| | | Human | |
| 49 | D9 | MpepMD | L[D]S[D]H[D] |
| | | (32-41) | LDGNL |
| | | (D-type | [D]S[D]D[D] |
| | | aa at | |
| | | N/C- | |
| | | termini)- | |
| | | Human | |
| 50 | D10 | MpepMD | L[D]S[D]H[D] |
| | | (32-41)- | LDGNLSDGGGGG |
| | | TAT | GGGGGYGRKKRRQ |
| | | (D-type | R[D]R[D]R[D] |
| | | aa at | |
| | | N/C- | |
| | | termini)- | |
| | | Human | |
| 51 | D11 | MpepMD | P[D]C[D]G[D] |
| | | (42-51) | PNRTD |
| | | (D-type | [D]L[D]G[D] |
| | | aa at | |
| | | N/C- | |
| | | termini)- | |
| | | Human | |
| 52 | D12 | MpepMD | P[D]C[D]G[D] |
| | | (42-51)- | PNRTDLGGGGGG |
| | | TAT | GGGGGYGRKKRRQ |
| | | (D-type | R[D]R[D]R[D] |
| | | aa at | |
| | | N/C- | |
| | | termini)- | |
| | | Human | |
| 53 | D13 | MpepMD | G[D]R[D]D[D] |
| | | (52-61) | SLCPP |
| | | (D-type | [D]T[D]G[D] |
| | | aa at | |
| | | N/C- | |
| | | termini)- | |
| | | Human | |
| 54 | D14 | MpepMD | G[D]R[D]D[D] |
| | | (52-61)- | SLCPPTGGGGGG |
| | | TAT | GGGGGGRKKRRQ |
| | | (D-type | R[D]R[D]R[D] |
| | | aa at | |
| | | N/C- | |
| | | termini)- | |
| | | Human | |
| 55 | D15 | MpepMD | L[D]S[D]H[D] |
| | | (32-61) | LDGNLSDPCGPN |
| | | (D-type | RTDLGGRDSLCPP |
| | | aa at | [D]T[D]G[D] |
| | | N/C- | |
| | | termini)- | |
| | | Human | |
| 56 | D16 | MpepMD | L[D]S[D]H[D] |
| | | (32-61)- | LDGNLSDPCGPN |
| | | TAT | RTDLGGRDSLCP |
| | | (D-type | PTGGGGGGGGGG |
| | | aa at | GYGRKKRRQR |
| | | N/C- | [D]R[D]R[D] |
| | | termini)- | |
| | | Human | |
| 57 | D17 | DpepDK | R[D]R[D]D[D] |
| | | (291-300) | PLVVA |
| | | (D-type | [D]A[D]L[D] |
| | | aa at | |
| | | N/C- | |
| | | termini)- | |
| | | Human | |
| 58 | D18 | DpepDK | R[D]R[D]D[D] |
| | | (291-300)- | PLVVAALGGGGGG |
| | | TAT | GGGGYGRKKRRQR |
| | | (D-type | [D]R[D]R[D] |
| | | aa at | |
| | | N/C- | |
| | | termini)- | |
| | | Human | |
| 59 | D19 | DpepDK | W[D]T[D]L[D] |
| | | (284-300) | VDIDRRDPLVVA |
| | | (D-type | [D]A[D]L[D] |
| | | aa at | |
| | | N/C- | |
| | | termini)- | |
| | | Human | |
| 60 | D20 | DpepDK | W[D]T[D]L[D] |
| | | (284-300)- | VDIDRRDPLVVA |
| | | TAT | ALGGGGGGGGGG |
| | | (D-type | YGRKKRRQR |
| | | aa at | [D]R[D]R[D] |
| | | N/C- | |
| | | termini)- | |
| | | Human | |
| 61 | D21 | KpepKD | E[D]A[D]L[D] |
| | | (297-312) | GSTSHSTAALS |
| | | (D-type | [D]S[D]Y[D] |
| | | aa at | |
| | | N/C- | |
| | | termini)- | |
| | | Human | |
| 62 | D22 | KpepKD | E[D]A[D]L[D] |
| | | (297-312)- | GSTSHSTAALSS |
| | | TAT | YGGGGGGGGGGY |
| | | (D-type | GRKKRRQR |
| | | aa at | [D]R[D]R[D] |
| | | N/C- | |
| | | termini)- | |
| | | Human | |
| 63 | D23 | Mpep | I[D]E[D]Q[D] |
| | | (358-382) | QNSTRIRQNTRD |
| | | Homo | HPSTANTVDRGG |
| | | (D-type | GGGGGGGGYGRK |
| | | aa at | KRRQR |
| | | N/C- | [D]R[D]R[D] |
| | | termini)- | |
| | | Human | |
| 64 | D24 | Kpep | V[D]Q[D]D[D] |
| | | (365-380) | PAYLRDIDGMNK |
| | | Homo | PVGGGGGGGGGG |
| | | (D-type | YGRKKRRQR |
| | | aa at | [D]R[D]R[D] |
| | | N/C- | |
| | | termini)- | |
| | | Human | |
| 65 | D25 | Kpep | D[D]I[D]D[D] |
| | | (372-380) | GMNKPVGGGGGG |
| | | Homo | GGGGYGRKKRRQR |
| | | (D-type | [D]R[D]R[D] |
| | | aa at | |
| | | N/C- | |
| | | termini)- | |
| | | Human | |
| 66 | D26 | Dpep | V[D]T[D]A[D] |
| | | (357-372) | CTPSDGPGGGAA |
| | | Homo | AGGGGGGGGGGY |
| | | (D-type | GRKKRRQR[ |
| | | aa at | D]R[D]R[D] |
| | | N/C- | |
| | | termini)- | |
| | | Human | |
| 67 | D27 | Dpep | R[D]P[D]D[D] |
| | | (339-372) | PSSFSRAREATA |
| | | Homo | RERVTACTPSDG |
| | | (D-type | PGGGAAAGGGGG |
| | | aa at | GGGGGYGRKKRR |
| | | N/C- | QR |
| | | termini)- | [D]R[D]R[D] |
| | | Human | |

(3) The prophylactic and/or therapeutic agent for the prevention and/or treatment of opioid tolerance or opioid dependence according to (1), wherein the dimer is a MOR and DOR heterodimer, or a KOR and DOR heterodimer.
(4) The prophylactic and/or therapeutic agent for the prevention and/or treatment of opioid tolerance or opioid dependence according to (3), wherein the peptide comprises any one amino acid sequence selected from the group consisting of the amino acid sequences of Sequence ID Numbers 1 to 30, amino acid sequences having one or more conservative amino acid substitutions in the amino acid sequence represented by sequence numbers 1 to 30, the amino acid sequences of Sequence ID Numbers 41 to 67 listed in Table B, amino acid sequences having one or more conservative amino acid substitutions in the amino acid sequences of Sequence ID Numbers 41 to 67 listed in Table B and amino acid sequences having at least 80% amino acid sequence identity with any of these amino acid sequences.
(5) The prophylactic and/or therapeutic agent for the prevention and/or treatment of opioid tolerance or opioid dependence according to (1), wherein the dimer is a homodimer of MOR, KOR or DOR.
(6) The prophylactic and/or therapeutic agent for the prevention and/or treatment of opioid tolerance or opioid dependence according to (5), wherein the peptide comprises any one amino acid sequence selected from the group consisting of the amino acid sequences of Sequence ID Numbers 31 to 40, amino acid sequences having one or more conservative amino acid substitutions in the amino acid sequences of Sequence ID Numbers 31 to 40 and amino acid sequences having at least 80% amino acid sequence identity with any of these amino acid sequences.
(7) A method of prevention and/or treatment of opioid tolerance or opioid dependence comprising administering a peptide which inhibits the formation of heterodimers or homodimers formed from one or two types of opioid receptor selected from the group consisting of the μ-type (MOR), the κ-type (KOR) and the δ-type (DOR) to a subject.
(8) The method of prevention and/or treatment of opioid tolerance or opioid dependence according to (7), wherein the peptide comprises any one amino acid sequence selected from the group consisting of the amino acid sequences of Sequence ID Numbers 1 to 40, amino acid sequences having one or more conservative amino acid substitutions in the amino acid sequences of Sequence ID Numbers 1 to 40, the amino acid sequences of Sequence ID Numbers 41 to 67 listed in Table B, amino acid sequences having one or more conservative amino acid substitutions in the amino acid sequences of Sequence ID Numbers 41 to 67 listed in Table B and amino acid sequences having at least 80% amino acid sequence identity with any of these amino acid sequences.
(9) The method of prevention and/or treatment of opioid tolerance or opioid dependence according to (7), wherein the peptide inhibits the formation of MOR and DOR heterodimers or KOR and DOR heterodimers.
(10) The method of prevention and/or treatment of opioid tolerance or opioid dependence according to (9), wherein the peptide comprises any one amino acid sequence selected from the group consisting of the amino acid sequences of Sequence ID Numbers 1 to 30, amino acid sequences having one or more conservative amino acid substitutions in the amino acid sequence represented by sequence numbers 1 to 30, the amino acid sequences of Sequence ID Numbers 41 to 67 listed in Table B, amino acid sequences having one or more conservative amino acid substitutions in the amino acid sequences of Sequence ID Numbers 41 to 67 listed in Table B and amino acid sequences having at least 80% amino acid sequence identity with any of these amino acid sequences.
(11) The method of prevention and/or treatment of opioid tolerance or opioid dependence according to (7), wherein the peptide inhibits the formation of homodimers of MOR, KOR or DOR.
(12) The method of prevention and/or treatment of opioid tolerance or opioid dependence according to (11), wherein the peptide comprises any one amino acid sequence selected from the group consisting of the amino acid sequences of Sequence ID Numbers 31 to 40, amino acid sequences having one or more conservative amino acid substitutions in the amino acid sequences of Sequence ID Numbers 31 to 40 and amino acid sequences having at least 80% amino acid sequence identity with any of these amino acid sequences.
(13) A peptide comprising any one amino acid sequence selected from the group consisting of the amino acid sequences of Sequence ID Numbers 1 to 40, amino acid sequences having one or more conservative amino acid substitutions in the amino acid sequences of Sequence ID Numbers 1 to 40, the amino acid sequences of Sequence ID Numbers 41 to 67 listed in Table B, amino acid sequences having one or more conservative amino acid substitutions in the amino acid sequences of Sequence ID Numbers 41 to 67 listed in Table B and amino acid sequences having at least 80% amino acid sequence identity with any of these amino acid sequences, wherein the peptide inhibits the formation of heterodimers or homodimers formed from one or two types of opioid receptor selected from the group consisting of the μ-type (MOR), κ-type (KOR), and δ-type (DOR).
(14) A peptide comprising any one amino acid sequence selected from the group consisting of the amino acid sequences of Sequence ID Numbers 1 to 30, amino acid sequences having one or more conservative amino acid substitutions in the amino acid sequences of Sequence ID Numbers 1 to 30, the amino acid sequences of Sequence ID Numbers 41 to 67 listed in Table B, amino acid sequences having one or more conservative amino acid substitutions in the amino acid sequences of Sequence ID Numbers 41 to 67 listed in Table B and amino acid sequences having at least 80% amino acid sequence identity with any of these amino acid sequences, wherein the peptide inhibits the formation of MOR and DOR heterodimers, or KOR and DOR heterodimers.
(15) A peptide comprising any one amino acid sequence selected from the group consisting of the amino acid sequences of Sequence ID Numbers 31 to 40, amino acid sequences having one or more conservative amino acid substitutions in the amino acid sequences of Sequence ID Numbers 31 to 40 and amino acid sequences having at least 80% amino acid sequence identity with any of these amino acid sequences, wherein the peptide inhibits the formation of homodimers of MOR, KOR or DOR.
(16) An agent for enhancing opioid analgesia comprising the peptide which inhibits the dimer formation of the opioid receptor, wherein the dimer is a heterodimer or a homodimer formed from one or two opioid receptors selected from the group consisting of the μ type (MOR), the κ type (KOR), and the δ type (DOR).
(17) A method of enhancing opioid analgesia comprising administering a peptide which inhibits the formation of heterodimers or homodimers formed from one or two types of opioid receptor selected from the group consisting of the μ-type (MOR), the κ-type (KOR) and the δ-type (DOR) in combination with opioids to a subject who needs to use opioids, who uses opioids, or who plans to use opioids.

### Advantageous Effects of Invention

The present inventions provide a method to treat opioid tolerance by using a soluble peptide-based inhibitor for dimerization of OPRs. Unlike the ligand- or kinase-based inhibitors, these peptide-based inhibitors suppress the morphine tolerance.

### BRIEF DESCRIPTION OF DRAWINGS

FIG. 1-1a OPRs in the PM form transient heterodimers with lifetimes of ˜250 ms. A typical image sequence of simultaneous two-color single fluorescent molecule observations, showing transient hetero-colocalisation and codiffusion of single molecules of DOR-SNAPf and MOR-Halo tagged with TMR-Star (green) and SaraFluor650T (magenta), respectively (see Methods).

FIG. 1-1b Colocalisation indices for correct and rotated overlays based on the pair cross-correlation functions (Extended Data FIG. 2), showing that DM and KD heterodimers form, whereas MK heterodimers do not. Throughout this study, we employed the following conventions: in box plots, horizontal bars, crosses, boxes, and whiskers indicate the median values, mean values, interquartile ranges (25-75%), and the 10-90% ranges, respectively; p values were obtained by using the Welch's two-tailed t test, except those for colocalisation lifetimes, for which the Brunner-Munzel test was employed; * and ns represent significant (p<0.05) and non-significant (p≥0.05) differences, respectively. Statistical parameters are summarised in Table C.

FIG. 1-1c Histograms showing the distributions of hetero (green and magenta)-colocalisation durations for correct and rotated overlays. The control histograms for rotated overlays (grey) were fitted by single exponential functions (black), providing the lifetime of the incidental overlap events between the magenta and green spots (t). The histograms for correct overlays (colors) were fitted by the sum of two exponential functions: The faster decay time (T1) was close to the lifetime of incidental overlaps (t), and the slower decay time provided the heterodimer lifetime (T2). The heterodimer lifetime after the correction for the photobleaching lifetime is shown in each box.

FIG. 1-1d Summary of the results shown in c and d.

FIG. 1-1e Comparison of the amino acid sequences among the three classical OPRs (rat) in the domains where the amino acid homologies are lower. Asterisks mark the identical amino acids among the three OPRs.

FIG. 1-2a EL3 domains are involved in the DK heterodimerisation. DK colocalisation indices (mean±SEM) for the pairs of WT-DOR (KOR) and the N/C-terminal deletion KOR (DOR) mutants.

FIG. 1-2b Colocalisation indices (mean±SEM) for the pairs of WT-KOR and the MOR mutants with the replacements of several domains by the corresponding DOR domains (testing whether the replacement enhances heterodimerisation; left), and for the pairs of WT-KOR and the DOR mutants with the replacements of several domains by the corresponding MOR domains (testing whether the replacement reduces heterodimerisation; right).

FIG. 1-2c Histograms showing the duration distributions for the pair of WT-KOR with the MOR mutant, in which its EL3 domain was replaced by DOR's EL3 domain (bottom). Histograms for the pairs of KD and MK are for the positive and negative controls (top and middle, respectively) and are the same as those shown in FIG. 1c.

FIG. 1-2d Effect of the addition of 1 μM soluble peptides with the sequences of the EL3 domains of three OPRs on DK heterodimerisation (colocalisation indices; mean±SEM).

FIG. 1-2c Colocalisation indices (mean±SEM) for the pairs of WT-KOR with TM1 and TM4 domains of DOR, showing that DOR's TM1 and TM4 domains moderately (TM1>TM4) interact with KOR.

FIG. 1-3a The N-terminal domains of DOR's aa22-42 and MOR's aa32-61 are involved in the DM heterodimerisation. DK colocalisation indices (mean±SEM) for the pairs of WT-MOR (DOR) and the systematically varied N-terminal deletion (and the C-terminal deletion) DOR (MOR) mutants.

FIG. 1-3b Histograms showing the duration distributions for the pair of WT-MOR and DOR's A22-42 deletion mutant (top, blue), and the pair of WT-DOR and MOR's A32-61 deletion mutant (bottom, magenta). Control histograms are the same as those shown in FIG. 1c.

FIG. 1-3c Colocalisation indices (mean±SEM) for the pairs of WT-MOR and the DOR mutants with the replacements of its TM domains and N-terminal domain by the corresponding KOR domains (testing whether the replacement reduces heterodimerisation; left), and for the pairs of WT-DOR and the MOR mutants with the replacements of TM1 and the N-terminal domain by the corresponding MOR domains (testing whether the replacement reduces heterodimerisation because the major DK interaction occurs at the EL3 domains; right).

FIG. 1-3d Effect of the addition of soluble peptides with the sequences of various parts of the N-terminal domain sequences of DOR and MOR (DpepDM and MpepDM, respectively) on DM heterodimerisation. The colocalisation indices at final concentrations of 10 μM (mean±SEM; left), those for Dpep(20-42)DM at various concentrations (the grey keys at 0 μM represent the data points obtained by using rotated overlays [control incidental colocalisation]), and the histogram showing the duration distribution for DM heterodimerisation in the presence of 10 PM Dpep(20-42)DM (blue). Control histograms are the same as those shown in FIG. 1c.

FIG. 1-3c The experimental design for examining the binding of SNAPf-tagged MOR's N-term+TM1 and TM1 to DOR and that of SNAPf-tagged DOR's TM4 (linked to the TM domain of LDL receptor to orient the TM4 correctly in the PM) to MOR.

FIG. 1-3f Colocalisation among TMs and OPRs. Colocalisation indices [mean±SEM].

FIG. 1-3g The abilities of the peptides to induce competitive dissociation of the DM heterodimers, using CHO-K1 cells expressing these molecules. Representative single-molecule images of DOR-mGFP (green), MOR-Halo (magenta), and MOR-TM1 (cyan) at similar expression levels of ˜0.7 spots/μm² are shown. DM colocalisation indices are plotted as a function of the protomer density ratio of the TM-peptide vs MOR.

FIG. 1-4a Effect of DM heterodimer blocker Dpep(20-42)DM on agonist-induced enhanced DM heterodimers, MOR signalling and trafficking, and morphine's analgesic effect and tolerance development in mouse. Schematic figure showing the basic mechanisms for the DOR-enhanced agonist-bound MOR signal and desensitisation, and the objectives of the experiments.

FIG. 1-4b he additions of the MOR agonist DAMGO and morphine (0.5 μM) enhanced and suppressed DM heterodimerisation, respectively (opposite effect). The presence of 10 μM Dpep(20-42)DM reduced DM heterodimers significantly for both DAMGO and morphine. The figure shows DM colocalisation indices in cells co-expressing MOR and DOR (mean±SEM).

FIG. 1-4c The additions of the MOR agonist DAMGO and morphine (0.5 μM) prolonged and shortened DM heterodimer lifetimes, respectively (opposite effect, but consistent with the colocalisation indices data in b). The presence of 10 μM Dpep(20-42)DM reduced DM heterodimer lifetimes significantly for both DAMGO and morphine.

FIG. 1-4d The time courses of MOR internalisation (mean±SEM; ≥20 cells for each condition), before and after the addition of 0.5 μM agonists (DAMGO, morphine, and SNC-80) in the presence and absence of 10 PM DpepDM.

FIG. 1-4c Experimental design for observing Ca2+ mobilisation after agonist addition (top). The Ca2+ mobilisation was monitored by the Fluo-4 fluorescence intensity, using CHO-K1 cells stably expressing Gqi5 (bottom). The cells with similar expression levels of OPRs (1.5±0.5 spots/μm²) were selected.

FIG. 1-4f The Ca²⁺ mobilisation was parametrised by using [FMax−Fb]/Fb.

FIG. 1-4g Schematic figure showing the subcutaneous implantation of an osmotic mini pump in mouse and intracerebroventricular administrations of Dpep (20-42) and morphine solutions.

FIG. 1-4h The tail flick test results on day 11, showing that morphine-induced analgesia was enhanced by the continuous administration of Dpep(20-42)DM (10 μg/day). The enhancing effect lasted for at least 120 min. Data are the mean±SEM: aCSF (n=7), peptide (n=9).

FIG. 1-4i Tolerance to morphine was demonstrated by the loss of the analgesic response on days 8 and 11 in the control aCSF-treated mice (AUC=area under the morphine concentration vs time curve). In contrast, the antinociceptive effect of morphine in DpepDM-treated mice was maintained at ˜70% of the initial effectiveness for 8-11 days. All experiments were randomised, performed by a blinded researcher, and then unblinded before statistical analysis. Data are the mean±SEM: aCSF (n=7), peptide (n=9).

FIG. 1-4j Cytotoxicity of DpepDM and DpepDM-TAT was not detectable when they were applied to Human bladder carcinoma cell line T24. The cytotoxicity of DpepDM and DpepDM-TAT was assessed in T24 cells after a 48-h exposure at concentrations (50 μM, 100 μM, and 200 μM) administered to the cells, using the lactate dehydrogenase (LDH) toxicity assay kit from Abcam according to the manufacturer's specification. Cytotoxicity was measured by subtracting LDH content in remaining viable cells from total LDH in untreated controls.

FIG. 1-4k Cytotoxicity of DpepDM and DpepDM-TAT was quite limited when they were applied to primary culture of hippocampal neurons. DpepDM and DpepDM-TAT toxicity was assessed using hippocampal neurons in culture (days in vitro [DIV] of 8 [DIV8], DIV12, and DIV14) after 48, 96, and 144-hour exposures at a concentration of 100 μM administered to the cells, using the LDH toxicity assay kit from Abcam according to the manufacturer's specification. Cytotoxicity was measured by subtracting LDH content in remaining viable cells from total LDH in untreated controls.

FIG. 2-1a OPRs in the PM form transient homodimers with lifetimes of 120-150 ms, without involving the N-terminal extracellular domain. Typical image sequence of simultaneous two-color single fluorescent molecule observations, showing transient homo-colocalization and co-diffusion of single molecules of SNAPf-KOR tagged with a mixture of two dyes, SNAP-CF 660R (magenta) and SNAP-Surface 549 (green).

FIG. 2-1b The pair cross-correlation functions (PCCFs), which are the distributions of the number of pairwise distances for all pairs of magenta and green SNAPf-KOR spots (after area normalization), plotted against the interparticle distance (mean±SEM; 18 movies). Green and grey bars indicate the results of the correct and control (180°-rotated) overlays of the simultaneously observed magenta and green images. The colocalization index is defined as (the mean of PCCF values for 0-50 and 50-100 nm)/(the mean of PCCF values for 400-450 and 450-500 nm) (see FIG. 2-s4).

FIG. 2-1c Colocalization indices for correct and rotated overlays, showing that the three OPRs undergo homo-colocalizations, that KOR exhibited more homo-colocalizations than MOR and DOR, and that the homodimerizations of all three OPRs were not affected by the deletions of the N-terminal extracellular domains. Throughout this study, we employed the following conventions: in box plots, horizontal bars, crosses, boxes, and whiskers indicate median values, mean values, interquartile ranges (25-75%), and 10-90% ranges, respectively; p values were obtained by Welch's two-tailed t test except for the Brunner-Munzel test used for colocalization lifetimes; * and ns represent significant (p<0.05) and non-significant (p≥0.05) differences, respectively; and statistical parameters are summarized in Supplementary Tables.

FIG. 2-1d Histograms showing the distributions of colocalization durations for correct and rotated overlays. The control histograms for rotated overlays (grey) were fitted by single exponential functions (black), providing the lifetime of the incidental overlap events between the magenta and green spots (t). The histograms for correct overlays (colors) were fitted by the sum of two exponential functions: The faster decay time (T1) was close to the lifetime of incidental overlaps (t), and the slower decay time provided the true colocalization lifetime or homodimer lifetime. The homodimer lifetimes after the corrections for the photobleaching lifetimes are shown in each box (summarized in Supplementary Table 2).

FIG. 2-2a C-terminal cytoplasmic domains are predominantly responsible for OPR homodimerization. Schematic figure showing the amino-acid sequences of the C-terminal cytoplasmic domains of three OPRs. The sequences surrounded by rectangles were extensively examined for their involvement in the OPR homodimerization.

FIG. 2-2b Colocalization indices for WT and various C-terminal cytoplasmic deletion mutants.

FIG. 2-2c Histograms showing the distributions of colocalization durations for various C-terminal cytoplasmic deletion mutants (only the results of correct overlays), as well as WT OPRs (the same as those shown in FIG. 11d; for both correct and rotated overlays). The exponential decay lifetimes of homodimers after the corrections for the photobleaching lifetime (2) are shown in each box and summarized in Supplementary Table 2.

FIG. 2-2d he colocalization indices of various C-terminal point mutants, indicating that the charged groups and prolines in the C-terminal cytoplasmic domains play important roles in OPR homodimerization.

FIG. 2-3a Specific C-terminal-domain peptides suppress OPR homodimerization both before and after agonist addition. Schematic figure showing the experimental design using mGFP-Xpeps expressed in the cell.

FIG. 2-3b Representative confocal image of CHO-K1 cells transfected with the cDNAs encoding SNAPf-KOR (not visible here) and mGFP-Kpep (green), costained with Live 650 Nuclear Stain (magenta).

FIG. 2-3c The OPR homo-colocalization index in each cell tends to decrease with an increase of the mGFP-Xpep concentration in the cytoplasm. The + keys at x=0 μM indicate the mean value of the colocalization index without mGFP-Xpep expression. The + keys at x=6 μM indicate the mean values of the colocalization indices averaged over all points in the range of 3.8-7.8 μM. Statistical parameters for these results are summarized in FIG. 2-s8a.

FIG. 2-3d Histograms showing the duration distributions of transient homodimers of WT SNAPf-OPRs in cells co-expressing 3.8-7.8 μM mGFP-Xpeps (only the results of correct overlays; the value for each bin is the mean of 18 cells), compared with those without co-expression (the same as those shown in FIG. 1d; for both correct and rotated overlays). The exponential decay lifetimes of homodimers after the corrections for the photobleaching lifetimes are shown in each box (t)) and summarized in Supplementary Table 3.

FIG. 2-4a Permeable Xpep-TAT suppress OPR homodimerization both before and after agonist addition. Schematic figure showing the experimental design, in which 20 μM FAM-Xpep-TATs were added to the cells (cytoplasmic concentrations in the range of 2.9-3.4 μM).

FIG. 2-4b A representative confocal image of CHO-K1 cells containing 3.4 μM FAM-Kpep-TAT.

FIG. 2-4c Agonist addition (0.2 μM) suppressed and enhanced the homodimerization of KOR and MOR/DOR, respectively (colocalization indices in c). The presence of ˜3 HM FAM-Xpep-TAT in the cytoplasm suppressed homodimerization both before and after the agonist addition. Agonist treatments: U-50488 for KOR, DAMGO for MOR, and SNC-80 for DOR, and all observations were performed between 0.5-3 min after the agonist addition. Statistical parameters and exponential homodimer lifetimes after corrections for photobleaching are summarized in FIG. 2-s8b and Table D, respectively.

FIG. 2-4d Agonist addition (0.2 μM) suppressed and enhanced the homodimerization of KOR and MOR/DOR, respectively (durations of colocalization events in d).

FIG. 2-4e Dependence of the D/M ratios (ratios of the numbers of protomers that exist as dimers vs. monomers) on the different expression levels of OPRs.

FIG. 2-4f Summary of KD, koff and kon for all three OPRs before and after the addition (3-5 min) of agonists and the D/M ratios (ratios of the numbers of protomers that exist as dimers vs. monomers) at different expression levels.

FIG. 2-5a OPR internalizations were greatly enhanced by the agonist addition, whereas the homodimerization-blocking peptides (FAM-Xpep-TATs and mGFP-Xpeps) hardly affected OPR internalizations in both the presence and absence of the agonist. Schematic figure showing the experimental design for observing the internalization of OPR (with possible recycling). Also see FIG. 2-s9.

FIG. 2-5b Time courses of SNAPf-OPR internalization in CHO-K1 cells (top) and T24 cells (bottom) (mean±SEM; 10 cells for each condition), before and after the addition of 0.2 μM agonist in the presence and absence of 2.9-3.4 μM FAM-Xpep-TATs and 3.8-7.8 μM mGFP-Xpeps. The time courses could be operationally fitted by single exponential functions, y=C*exp(−t/τ₀)+ (1−C), providing the fraction of OPR molecules with PM residency lifetimes longer than the detection limit in this experimental design (1-C; for observations up to 35 min), as well as the fraction of OPR molecules, for which the residency time (τ₀) is measurable (C). These parameters are summarized in Table D.

FIG. 2-6a Xpeps enhanced, did not affect, or reduced the agonist-induced Ca²⁺ mobilization by KOR, MOR, and DOR, respectively. Schematic figure showing the experimental design for observing the Ca²⁺ mobilization after the agonist stimulation. The Ca²⁺ mobilization was monitored by the Rhod-3 fluorescence intensity, using CHO-K1 cells stably expressing Gqi5 (see the caption to FIG. 2-s1b and Methods). The bottom table shows typical fluorescence images of Rhod-3 in cells (orange rectangles=ROI) The graph on the right shows the time-dependent changes of the Rhod-3 signal intensity in the ROI, showing the changes in the cytosolic Ca²⁺ concentration after the agonist stimulation. Cells with similar expression levels of OPRs (0.5-1.5 spots/μm²) were selected.

FIG. 2-6b Ca²⁺ mobilization parametrized by using [FMax−Fb]/Fb (see c) within 75 s after the addition of the stimulants.

FIG. 2-s1a Sequence comparison among the three classical OPRs (rat), the test for the function of the SNAPf-tagged OPRs, and the IUPred2 score plots suggesting the intrinsically disordered conformations of the C-terminal cytoplasmic domains of OPR.

FIG. 2-s1b Schematic figure showing the experimental design for testing whether the OPR conjugated with the tag protein SNAPf at the N-terminal ectodomain can function as well as the wild-type OPR (Methods). The CHO-K1 cells stably expressing Gqi5, the Gaq with the final five amino-acid sequence (ECGLY) replaced by that of Gαi2 (DCGLF)¹⁻³, were generated and then transfected with the cDNAs encoding the SNAPf-linked OPR or the wild-type OPR conjugated to mCherry by way of the 2A-peptide linker (mCherry-2A-[WT]OPR). Cells expressing the SNAPf-OPR in the range of 0.5-1.5 spots/μm² and mCherry at detectable levels using epi-illumination were employed. The Ca²⁺ mobilization was monitored by the fluorescence intensity of the Ca²⁺-sensitive fluorescent dye Fluo-4. For the agonist stimulation, 0.2 μM U-50488, DAMGO, and SNC-80 were used for KOR, MOR, and DOR, respectively.

FIG. 2-s1c Ca²⁺ mobilization parametrized by using [F_{Max}−F_{b}]/F_{b}, where F_{Max} is the maximal Fluo-4 signal intensity within 75 s after the addition of the stimulants and F_{b} is the baseline intensity⁴.

FIG. 2-s1d The IUPred2 score, a parameter indicating the level of intrinsic conformational disorder, plotted against the full amino-acid sequence of the OPR. The sequences with IUPred scores greater than 0.5 for some lengths are often considered intrinsically disordered. The C-terminal cytoplasmic domains exhibited propensities of being intrinsically disordered.

FIG. 2-s1e The IUPred2 scores plotted against the sequences in the C-terminal cytoplasmic region of the WT- and mutant-OPRs. The IUPred2 scores of the mutants suggested less disordered conformations.

FIG. 2-s2a SNAPf bound to the N-terminus of a monomeric TM protein, CD47, expressed in the PM of CHO-K1 cells could be labeled with the fluorescent membrane-impermeable SNAP ligands, SNAP-Surface 549 and SNAP-CF660R, with ≥70% efficiencies. Schematic figure showing the monomeric 5-pass TM protein CD47⁵ linked to both the SNAPf-tag and mGFP-tag at its N- and C-termini, respectively (SNAPf-CD47-mGFP), expressed in the PM. Since OPRs form homodimers, their labeling efficiency was difficult to evaluate, and therefore CD47 was employed.

FIG. 2-s2b Typical simultaneous two-color, single-molecule images of SNAPf-CD47-mGFP in the PM after the incubation with 0.6 μM SNAP-Surface 549-conjugated ligand at 37° C. for 30 min (among 20 cells).

FIG. 2-s2c Plot showing the percentages of fluorescent mGFP spots (of SNAPf-CD47-mGFP) that were colocalized by the SNAP-dye spots (mean±SEM for n=20 cells) as a function of time after the addition of the SNAP-Surface 549-conjugated ligand. The plot could be fitted by a single exponential function, providing the labeling efficiency of the SNAPf protein tag on CD47 with SNAP-Surface 549 (74±9%). Similar results were obtained with SNAP-CF660R (70±8%). Note that not every mGFP molecule is fluorescent, but we identify the existence of a SNAPf-CD47-mGFP molecule if the mGFP is fluorescent, and then we evaluate how many percentages of the identified mGFP molecules were colocalized by the fluorescence spots of SNAP-ligands. This way, the labeling efficiencies of the SNAP-tag can be evaluated.

FIG. 2-s3 Transient homodimerization events of the OPR in the PM are detectable by single-color, single-molecule imaging⁷⁻⁸. The homodimerization lifetimes are on the order of ˜0.1 s, consistent with the simultaneous two-color results. Typical image sequence (every 33 ms) (a) and trajectories (b) of three diffusing SNAPf-KOR molecules (tagged with SNAP-Surface 549) undergoing two transient homodimerization events. In b, circles with A, B, and C indicate the location of three molecules in the first video frame (see the fluorescent spots in a). After the first colocalization of molecules A and B lasting for 133 ms, the second colocalization of the [A or B] molecule with the C molecule started after 333 ms (11th frame), and lasted for 66 ms. Due to the single-color labeling, after the first colocalization event, we cannot tell which molecules are A and B, and thus we state the molecule [A or B].

FIG. 2-s4a Definition of the colocalization index based on the spatial pair cross-correlation function (PCCF) in the form of the histogram. From simultaneously recorded single-molecule movies with different colors (say green and magenta), the spatial pair cross-correlation function (PCCF) was determined. First, a region of interest (ROI) with a rectangular shape greater than 10 μm on each side is selected (to avoid the effect of edges, employing the ROI of this size or greater would be useful). The distances between all pairs of green and magenta spots in the ROI in the simultaneously obtained video frames are measured, and this is repeated for all of the video frames (100 frames×18 movies [18 cells]; Supplementary Table 1). Using this set of interparticle distances, the distribution of the number of distances normalized for the unit area (i.e., the number densities at a given distance) for all pairs (mean±SEM for all movies used for the analysis) is plotted against the distance. A bin size of 50 nm and a maximal distance of 500 nm were used throughout this study (see c).

FIG. 2-s4b As a control, the green image is superimposed on the 180 degree-rotated magenta image (green and magenta are interchangeable), which is called the “180°-rotated overlay” (vs. the correct overlay).

FIG. 2-s4c Definition of the colocalization index. The histogram shows a schematic example of the PCCF, determined by the method described in (a). The colocalization index is defined as [the mean of the PCCF value for 0-50 and that for 50-100 nm; shown by blue highlighting] divided by [the mean of the PCCF value for 400-450 and that for 450-500 nm; shown by orange highlighting]. When no significant colocalization exists, the colocalization index is 1, and with an increase of colocalizations, the index increases. For the actual functional form describing the PCCF based on the number densities of spots in the images and the dimer-monomer dissociation equilibrium constant K_{D}.

FIG. 2-s5a The extent to which the number density of the fluorescent spots in the images (expression levels of molecules) influences the PCCF and colocalization index, as well as K_{D}. (a) Typical distributions of the fluorescent spots in the square ROI (10 μm×10 μm) in the two-color images, generated by Monte-Carlo simulations. The numbers of magenta and green spots are approximately the same, and the number densities of the spots shown are the total numbers of the magenta and green spots. For the detailed method of the simulations, see the end of this caption. This figure represents a snapshot, showing the spatial distributions of the fluorescent spots, at the end of step 6 in the simulation steps in the method part at the end of this caption.

FIG. 2-s5b (b) Comparison of the simulation results (+) with theoretically obtained PCCFs (solid curves), to find the applicability limitations of the theoretically obtained PCCF. Four primary parameters, K_{D}, σ (error for single-molecule localization and overlay of two-color images), the total spot density (of green and magenta molecules), and the labeling efficiency, were systematically varied, and the Monte-Carlo simulations were performed. From the spatial distributions of the spots, the experimental PCCF (obtained by simulation) was directly calculated (+; see step 8-1 in the method at the end of this caption). Meanwhile, the theoretical PCCFs (solid curves) and NAB were calculated with the given sN_{TA}, sN_{TB}, and K_{D} using Eqs. The theoretical PCCFs agree well with the simulated experimental PCCFs in broad ranges of the parameters, except for the case in which the spot density is 5 spots/μm² (bottom-left). Under these conditions, the incidental colocalizations of more than three molecules, which we neglected in our theory, would become important. However, spot densities of ≥5 spots/μm² are unrealistic for single-molecule imaging (individual single molecules are not distinctly detectable).

FIG. 2-s5c (c) The dependence of the colocalization index on the number density of the spots in the image and K_{D} obtained by Monte-Carlo simulations. The range of the total spot densities employed in the present research (0.5-1.5 spots/μm²) is highlighted in yellow (top). In the K_{D} ranges found for GPCRs; i.e., 3.6 copies/μm² for formyl-peptide receptor⁶, 1.6 copies/μm² for β₂-adrenergic receptor⁸, as well as 2.68, 7.31, and 7.91 copies/μm² for KOR, MOR, and DOR, respectively, found in (g), the colocalization index weakly depends on the spot number density in the experimental range employed here (0.5-1.5 spots/μm²). Also see f.

FIG. 2-s5d (d) Experimental data showing the dependence of the colocalization index of MOR on the spot number density in the image (spots/μm²). Each magenta key represents the colocalization index and the spot number density for each examined cell (n=120 cells). The plus marks placed at spot number densities of 0.475, 0.95, and 1.425 spots/μm² indicate the mean colocalization index values for the cells with spot number densities in the ranges of 0.25-0.7, 0.7-1.2, and 1.2-1.65 spots/μm², respectively. This result shows that the variations of the spot number density in the range of 0.5˜1.5 spots/μm² would not be the limiting factor for the accuracy in determining the colocalization index of MOR.

FIG. 2-s5e (c) Dependence of the fraction of incidentally colocalized molecules on the number density of molecules, randomly distributed in the ROI (10 μm×10 μm), using the threshold distance R_{Th} of 200 nm (diffraction limit). In our experimental range (0.5˜1.5 spots/μm²), less than 11% of the molecules were colocalized incidentally.

FIG. 2-s5f (f) Comparison of the true K_{D} given for the simulation and the K_{D} estimated to the simulated spot distributions (with the localization+overlay error of 140 nm and labeling efficiency of 0.7). The dependence of the K_{D} estimated by the theory on the total spot number density (simulation) is shown. The range of the total spot densities employed in the present research (0.5-1.5 spots/μm²) is highlighted in yellow. In this range, the estimated K_{D} using the method described here is underestimated (higher affinities) by 6.4, 5.2, and 3.6% for the K_{D} values of 1, 3, and 6 copies/μm², respectively. The underestimation is worse with an increase in the spot number density, suggesting that this is caused by neglecting the incidental colocalization of more than three molecules in our theory, consistent with the results shown in c.

FIG. 2-s5g (g) Experimental PCCFs for KOR, MOR, and DOR (the PCCF for KOR is the same as that shown in FIG. 2-1b) fitted with theoretical PCCFs (the total numbers of fluorescent spots, sN_{TA} and sN_{TB}, were determined from experiments, and a labeling efficiency of 0.7 was obtained by independent experiments; see FIG. 2-s2c), using K_{D} and σ as fitting parameters. The magenta and purple graphs indicate the best-fit functions, and their K_{D} values are shown (these colors are matched with those in c). The PCCFs with K_{D} values of 1 and 10 (other fit parameters are fixed at the same values for the best-fit functions) are also shown for comparison.

FIG. 2-s6 Distributions of photobleaching lifetimes of single dye molecules, SNAP-Surface 549 and SNAP-CF660R, adsorbed on the coverslip of the glass-base dish. These histograms could be fitted by single exponential functions, and the decay time constants provided the photobleaching lifetimes of SNAP-Surface 549 and SNAP-CF660R of 16.3±1.2 and 7.8±0.6 s, respectively.

FIG. 2-s7 Calibration curves (straight lines) for evaluating the concentrations of mGFP-Xpeps and FAM-Xpep-TATs in the cytoplasm. Purified mGFP and FAM-Kpep-TAT were dissolved in Ham's F12 observation medium at various concentrations. The solutions were placed in glass-base dishes, and then confocal images were captured with a focus at 5 μm above the cover-glass surface. The confocal microscopy was performed with the same microscope station used for single-molecule imaging at 37° C., equipped with an Olympus SR10 spinning-disc confocal super-resolution unit (see Methods). The fluorescence intensities in the ROI of a 10 μm×10 μm square were plotted as a function of the concentration of mGFP (a) or FAM-Kpep-TAT (b) (n=three dishes). The ranges of the fluorescence intensities and the peptide concentrations in the cells used for the actual observations are shown by grey areas.

FIG. 2-s8a Statistical analysis results of the data shown in FIG. 2-3c (a) and FIG. 2-4c (b).

FIG. 2-s8b Statistical analysis results of the data shown in FIG. 2-3c (a) and FIG. 2-4c (b).

FIG. 2-s9 Images of SNAPf-DOR labeled with SNAP-Surface 549 before and after the addition of the membrane-impermeable fluorescence quencher Mn³⁺-TSP, in the absence of the agonist SNC-80 and 30 min after the agonist addition. The fluorescent spots after the addition of the quencher represent the internalized SNAPf-DOR molecules (labeled with SNAP-Surface 549).

FIG. 2-s10a Schematic structures of the cDNA constructs used in this study, showing the multiple cloning sites, inserted tag proteins, linkers, and target proteins.

FIG. 2-s10b Schematic structures of the cDNA constructs used in this study, showing the multiple cloning sites, inserted tag proteins, linkers, and target proteins.

FIG. 2-s10c Schematic structures of the cDNA constructs used in this study, showing the multiple cloning sites, inserted tag proteins, linkers, and target proteins.

### DESCRIPTION OF EMBODIMENTS

Unless otherwise noted, all terms in the present invention have the same meaning as commonly understood by one with ordinary skill in the art to which this disclosure belongs. The singular terms “a,” “an,” and “the” include plural referents unless context indicates otherwise. Similarly, the word “or” is intended to include “and” unless the context indicates otherwise. In this specification, molecular biological techniques can be performed by methods described in general experimental manuals known to those skilled in the art or by methods similar thereto, unless otherwise specified.

The opioids are important for clinical pain management, but repeated use reduces their analgesic effect, called “tolerance”, and thus, long-term opioid therapy is associated with increased risk of abuse, fatal overdose, dependence, and addiction, leading to serious clinical and social problems. Therefore, methods to suppress tolerance development to opioids are badly needed.

The present invention is to solve these problems. Inventors invented peptide drugs for suppressing tolerance development to morphine, the gold standard of opioid-based analgesia, and possibly to other analgesics. These peptides have not been known so far. They work by modulating the opioid receptor functions. One of the peptides was found to suppress the tolerance development to morphine in mouse.

The opioids work by binding to opioid receptors (OPRs) located on the surface of neurons in the neuronal circuits that regulate pain perception, hedonic homeostasis, mood, and well-being. Three classical OPRs, called μ-type, κ-type, and δ-type-OPRs (MOR, KOR, and DOR, respectively) exist in our body.

Inventors found homo- and hetero-dimerization of OPRs, the methods to block their dimerization using peptide drugs, and that such inhibitions modulate the OPRs' downstream signals and OPR internalization, leading to this invention.

(Prophylactic and/or Therapeutic Agent for the Prevention and/or Treatment of Opioid Tolerance or Opioid Dependence)

In one embodiment, the present application includes a prophylactic and/or therapeutic agent for the prevention and/or treatment of opioid tolerance or opioid dependence comprising a peptide which inhibits the formation of a dimer, wherein the dimer is a heterodimer or a homodimer formed from one or two opioid receptors selected from the group consisting of the μ type (MOR), the κ type (KOR), and the δ type (DOR).

The peptide preferably comprises any one amino acid sequence selected from the group consisting of the amino acid sequences of Sequence ID Numbers 1 to 40 and the amino acid sequences of Sequence ID Numbers 41 to 67 listed in Table B. The peptide more preferably comprises any one amino acid sequence selected from the group consisting of the amino acid sequences of Sequence ID Numbers 2 to 8, 11 to 13, 15, 17 to 40 and the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 1 to 40 and in the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 2 to 8, 11 to 13, 15 and 17 to 40 and in the amino acid sequences of Sequence ID Numbers 41 to 67 listed in Table B. The peptide may comprise any one amino acid sequence having at least 80%, 90%, 95%, or 99% amino acid sequence identity with any of these amino acid sequences.

TABLE 2
Table A. Dimer blockers

| SQ | | |
|---|---|---|
| ID | | Amino Acid |
| No. | | Sequence |
| 1 | DpepDM(1-20) | MEPVPSARAELQ |
| | | FSLLANVS |
| 2 | DpepDM(20-27) | SDTFPSAF |
| 3 | DpepDM(20-27) + | SDTFPSAFD |
| | Asp (D) | |
| 4 | DpepDM(20-27) + | SDTFPSAFSSS |
| | SerX3 (SSS) | |
| 5 | DpepDM(20-37) | SDTFPSAFPSA |
| | | SANASGS |
| 6 | DpepDM(20-37) + | SDTFPSAFPSA |
| | Asp (D) | SANASGSD |
| 7 | DpepDM(33-42) | NASGSPGARS |
| 8 | DpepDM(20-42) | SDTFPSAFPSA |
| | | SANASGSPGAR |
| | | S |
| 9 | DpepDM(20-42) | SPSSFAPGSNG |
| | Scrambled seq. | ATADFSPASAS |
| | | R |
| 10 | MpepMD(1-31) | MDSSTGPGNTS |
| | | DCSDPLAQASC |
| | | SPAPGSWLN |
| 11 | MpepMD(32-41) | LSHVDGNQSD |
| 12 | MpepMD(42-51) | PCGLNRTGLG |
| 13 | MpepMD(52-61) | GNDSLCPQTG |
| 14 | MpepMD(62-70) | SPSMVTAIT |
| 15 | MpepMD(32-61) | LSHVDGNQSDP |
| | | CGLNRTGLGGN |
| | | DSLCPQTG |
| 16 | MpepMD(32-61) | CVPRCQGSNGQ |
| | Scrambled seq. | HDGGTSGGNPD |
| | | DNLLSLTL |
| 17 | DpepDK(291-300) | RRDPLVVAAL |
| 18 | DpepDK(284-300) | WTLVDINRRDP |
| | | LVVAAL |
| 19 | DpepKD (297-312) | EALGSTSHSTA |
| | | VLSSY |
| 20 | DpepDM(20-27)- | SDAYPSAC |
| | Human | |
| 21 | DpepDM(20-37)- | SDAYPSACPSA |
| | Human | GANASGP |
| 22 | DpepDM(33-42)- | NASGPPGARS |
| | Human | |
| 23 | DpepDM(20-42)- | SDAYPSACPSA |
| | Human | GANASGPPGAR |
| | | S |
| 24 | MpepMD(32-41)- | LSHLDGNLSD |
| | Human | |
| 25 | MpepMD(42-51)- | PCGPNRTDLG |
| | Human | |
| 26 | MpepMD(52-61)- | GRDSLCPPTG |
| | Human | |
| 27 | MpepMD(32-61)- | LSHLDGNLSDP |
| | Human | CGPNRTDLGGR |
| | | DSLCPPTG |
| 28 | DpepDK(291-300)- | RRDPLVVAAL |
| | Human | |
| 29 | DpepDK(284-300)- | WTLVDIDRRDP |
| | Human | LVVAAL |
| 30 | KpepKD (297-312)- | EALGSTSHSTA |
| | Human | ALSSY |
| 31 | Mpep(358-382) | EQQNSTRVRQN |
| | Homo | TREHPSTANTV |
| | | DRGGGGGGGGG |
| | | GYGRKKRRQRR |
| | | R |
| 32 | Kpep(365-380) | QDPASMRDVGG |
| | Homo | MNKPVGGGGGG |
| | | GGGGYGRKKRR |
| | | QRRR |
| 33 | Kpep(372-380) | DVGGMNKPVGG |
| | Homo | GGGGGGGGYGR |
| | | KKRRQRRR |
| 34 | Dpep(357-372) | VTACTPSDGPG |
| | Homo | GGAAAGGGGGG |
| | | GGGGYGRKKRR |
| | | QRRR |
| 35 | Dpep(339-372) | GQEPGSLRRPR |
| | Homo | QATARERVTAC |
| | | TPSDGPGGGAA |
| | | AGGGGGGGGGG |
| | | YGRKKRRQRRR |
| 36 | Mpep(358-382) | IEQQNSTRIRQ |
| | Homo-Human | NTRDHPSTANT |
| | | VDRGGGGGGGG |
| | | GGYGRKKRRQR |
| | | RR |
| 37 | Kpep(365-380) | VQDPAYLRDID |
| | Homo-Human | GMNKPVGGGGG |
| | | GGGGGYGRKKR |
| | | RQRRR |
| 38 | Kpep(372-380) | DIDGMNKPVGG |
| | Homo-Human | GGGGGGGGYGR |
| | | KKRRQRRR |
| 39 | Dpep(365-372) | VTACTPSDGPG |
| | Homo-Human | GGAAAGGGGGG |
| | | GGGGYGRKKRR |
| | | QRRR |
| 40 | Dpep(339-372) | RPDPSSFSRAR |
| | Homo-Human | EATARERVTAC |
| | | TPSDGPGGGAA |
| | | AGGGGGGGGGG |
| | | YGRKKRRQRRR |

TABLE 3
Table B. Dimer blockers (comprising
D-type-amino acid)

| SQ | | | |
|---|---|---|---|
| ID | | | Amino acid |
| No. | No. | | sequence |
| 41 | D1 | DpepDM | S[D]D[D]T[D] |
| | | (20-42) | FPSAFPSASANA |
| | | (D-type | SGSPGA |
| | | aa at | [D]R[D]S[D] |
| | | N/C- | |
| | | termini) | |
| 42 | D2 | DpepDM | S[D]D[D]T[D] |
| | | (20-42)- | FPSAFPSASANA |
| | | TAT | SGSPGARSGGGGG |
| | | (D-type | GGGGGYGRKKRRQR |
| | | aa at | [D]R[D]R[D] |
| | | N/C- | |
| | | termini) | |
| 43 | D3 | DpepDM | S[D]D[D]A[D] |
| | | (20-27) | YPS |
| | | (D-type | [D]A[D]C[D] |
| | | aa at | |
| | | N/C- | |
| | | termini)- | |
| | | Human | |
| 44 | D4 | DpepDM | S[D]D[D]A[D] |
| | | (20-27)- | YPSACGGGGGGG |
| | | TAT | GGGYGRKKRRQR |
| | | (D-type | [D]R[D]R[D] |
| | | aa at | |
| | | N/C- | |
| | | termini)- | |
| | | Human | |
| 45 | D5 | DpepDM | S[D]D[D]A[D] |
| | | (20-37) | YPSACPSAGANAS |
| | | (D-type | [D]G[D]P[D] |
| | | aa at | |
| | | N/C- | |
| | | termini)- | |
| | | Human | |
| 46 | D6 | DpepDM | S[D]D[D]A[D] |
| | | (20-37)- | YPSACPSAGANA |
| | | TAT | SGPGGGGGGGGG |
| | | (D-type | GYGRKKRRQR |
| | | aa at | [D]R[D]R[D] |
| | | N/C- | |
| | | termini)- | |
| | | Human | |
| 47 | D7 | DpepDM | S[D]D[D]A[D] |
| | | (20-42) | YPSACPSAGANA |
| | | (D-type | SGPPGA |
| | | aa at | [D]R[D]S[D] |
| | | N/C- | |
| | | termini)- | |
| | | Human | |
| 48 | D8 | DpepDM | S[D]D[D]A[D] |
| | | (20-42)- | YPSACPSAGANA |
| | | TAT | SGPPGARSGGGGGG |
| | | (D-type | GGGGYGRKKRRQR |
| | | aa at | [D]R[D]R[D] |
| | | N/C- | |
| | | termini)- | |
| | | Human | |
| 49 | D9 | MpepMD | L[D]S[D]H[D] |
| | | (32-41) | LDGNL |
| | | (D-type | [D]S[D]D[D] |
| | | aa at | |
| | | N/C- | |
| | | termini)- | |
| | | Human | |
| 50 | D10 | MpepMD | L[D]S[D]H[D] |
| | | (32-41)- | LDGNLSDGGGGG |
| | | TAT | GGGGGYGRKKRRQ |
| | | (D-type | R[D]R[D]R[D] |
| | | aa at | |
| | | N/C- | |
| | | termini)- | |
| | | Human | |
| 51 | D11 | MpepMD | P[D]C[D]G[D] |
| | | (42-51) | PNRTD |
| | | (D-type | [D]L[D]G[D] |
| | | aa at | |
| | | N/C- | |
| | | termini)- | |
| | | Human | |
| 52 | D12 | MpepMD | P[D]C[D]G[D] |
| | | (42-51)- | PNRTDLGGGGGG |
| | | TAT | GGGGGYGRKKRRQ |
| | | (D-type | R[D]R[D]R[D] |
| | | aa at | |
| | | N/C- | |
| | | termini)- | |
| | | Human | |
| 53 | D13 | MpepMD | G[D]R[D]D[D] |
| | | (52-61) | SLCPP |
| | | (D-type | [D]T[D]G[D] |
| | | aa at | |
| | | N/C- | |
| | | termini)- | |
| | | Human | |
| 54 | D14 | MpepMD | G[D]R[D]D[D] |
| | | (52-61)- | SLCPPTGGGGGG |
| | | TAT | GGGGGYGRKKRRQ |
| | | (D-type | R[D]R[D]R[D] |
| | | aa at | |
| | | N/C- | |
| | | termini)- | |
| | | Human | |
| 55 | D15 | MpepMD | L[D]S[D]H[D] |
| | | (32-61) | LDGNLSDPCGPN |
| | | (D-type | RTDLGGRDSLCPP |
| | | aa at | [D]T[D]G[D] |
| | | N/C- | |
| | | termini)- | |
| | | Human | |
| 56 | D16 | MpepMD | L[D]S[D]H[D] |
| | | (32-61)- | LDGNLSDPCGPN |
| | | TAT | RTDLGGRDSLCPPT |
| | | (D-type | GGGGGGGGGGGYG |
| | | aa at | RKKRRQR |
| | | N/C- | [D]R[D]R[D] |
| | | termini)- | |
| | | Human | |
| 57 | D17 | DpepDK | R[D]R[D]D[D] |
| | | (291-300) | PLVVA[ |
| | | (D-type | D]A[D]L[D] |
| | | aa at | |
| | | N/C- | |
| | | termini)- | |
| | | Human | |
| 58 | D18 | DpepDK | R[D]R[D]D[D] |
| | | (291-300)- | PLVVAALGGGGGG |
| | | TAT | GGGGYGRKKRRQR |
| | | (D-type | [D]R[D]R[D] |
| | | aa at | |
| | | N/C- | |
| | | termini)- | |
| | | Human | |
| 59 | D19 | DpepDK | W[D]T[D]L[D] |
| | | (284-300) | VDIDRRDPLVVA |
| | | (D-type | [D]A[D]L[D] |
| | | aa at | |
| | | N/C- | |
| | | termini)- | |
| | | Human | |
| 60 | D20 | DpepDK | W[D]T[D]L[D] |
| | | (284-300)- | VDIDRRDPLVVA |
| | | TAT | ALGGGGGGGGGG |
| | | (D-type | YGRKKRRQR |
| | | aa at | [D]R[D]R[D] |
| | | N/C- | |
| | | termini)- | |
| | | Human | |
| 61 | D21 | KpepKD | E[D]A[D]L[D] |
| | | (297-312) | GSTSHSTAALS |
| | | (D-type | [D]S[D]Y[D] |
| | | aa at | |
| | | N/C- | |
| | | termini)- | |
| | | Human | |
| 62 | D22 | KpepKD | E[D]A[D]L[D] |
| | | (297-312)- | GSTSHSTAALSS |
| | | TAT | YGGGGGGGGGGY |
| | | (D-type | GRKKRRQR |
| | | aa at | [D]R[D]R[D] |
| | | N/C- | |
| | | termini)- | |
| | | Human | |
| 63 | D23 | Mpep | I[D]E[D]Q[D] |
| | | (358-382) | QNSTRIRQNTRD |
| | | Homo | HPSTANTVDRGG |
| | | (D-type | GGGGGGGGYGRK |
| | | aa at | KRRQR |
| | | N/C- | [D]R[D]R[D] |
| | | termini)- | |
| | | Human | |
| 64 | D24 | Kpep | V[D]Q[D]D[D] |
| | | (365-380) | PAYLRDIDGMNK |
| | | Homo | PVGGGGGGGGGG |
| | | (D-type | YGRKKRRQR |
| | | aa at | [D]R[D]R[D] |
| | | N/C- | |
| | | termini)- | |
| | | Human | |
| 65 | D25 | Kpep | D[D]I[D]D[D] |
| | | (372-380) | GMNKPVGGGGGG |
| | | Homo | GGGGYGRKKRRQR |
| | | (D-type | [D]R[D]R[D] |
| | | aa at | |
| | | N/C- | |
| | | termini)- | |
| | | Human | |
| 66 | D26 | Dpep | V[D]T[D]A[D] |
| | | (357-372) | CTPSDGPGGGAA |
| | | Homo | AGGGGGGGGGGY |
| | | (D-type | GRKKRRQR |
| | | aa at | [D]R[D]R[D] |
| | | N/C- | |
| | | termini)- | |
| | | Human | |
| 67 | D27 | Dpep | R[D]P[D]D[D] |
| | | (339-372) | PSSFSRAREATA |
| | | Homo | RERVTACTPSDG |
| | | (D-type | PGGGAAAGGGGG |
| | | aa at | GGGGGYGRKKRR |
| | | N/C- | QR |
| | | termini)- | [D]R[D]R[D] |
| | | Human | |

In the present application. “conservative amino acid substitutions” means as follows;
(i) Replacing the positively charged amino acids with other positively charged amino acids (K, R, H) and with the amino acids with polar uncharged amino acids (S, T, N, Q) and vice versa
(ii) Replacing the negatively charged amino acids with other negatively charged amino acids (D, E) and with the amino acids with polar uncharged amino acids (S, T, N, Q) and vice versa.
(iii) Replacing the amino acids with polar uncharged amino acids (S, T, N, Q) among them and replacing them with C, G, and P (containing side chains with slight hydrophilicity) and vice versa.
(iv) Replacing the amino acids C, G, and P among them.
(v) Replacing the non-polar (hydrophobic) amino acids with other non-polar amino acids (A, V, I, L, M, F, Y, W) and with amino acids containing side chains with slight hydrophilicity (C, G, P) and vice versa.

In the present application, amino acids may be either natural L-type or artificial D-type. D-type amino acids can be used at the N- and C-termini of the peptides to reduce the decomposition rate of the peptide in the body. In the present application, the amino acid sequence of the chain peptide is described according to the conventional manner of peptide indication with the N-terminal side on the left side and the C-terminal side on the right side. In addition, each amino acid symbol with symbol [D] immediately following the amino acid sequence indicates a D form of the amino acid, and each amino acid symbol without symbol [D] immediately following the amino acid sequence indicates an L form of the amino acid, unless contrary to the context.

In the present invention, the peptide may be modified at a part or all of the amino acid residues in its amino acid sequence. Such modified peptides may be prepared by any method known in the art. For example, the modified peptide may be prepared by modification such as esterification, alkylation, halogenation, phosphorylation, sulfonation, or amidation of the functional group of the side chain of the amino acid residue(s) constituting the peptide.

In the present invention, the peptide may be fused with, conjugated with or added to another substance. The peptide may be conjugated with or bound to a certain substance via a chemical such as a cross-linking agent, or via an agent suitable for linking to a side chain of an amino acid, or by a synthetic chemical or genetic engineering technique to the N-terminus and/or C-terminus of the peptide. Examples of such a substance for improving blood half-life can include polyalkylene glycol molecules such as polyethylene glycol (PEG); a fatty acid molecule such as hydroxyethyl starch or palmitic acid; an Fc region of immunoglobulin; a CH3 domain of immunoglobulin; a CH4 domain of immunoglobulin; albumin or a fragment thereof; an albumin-binding peptide; an albumin-binding protein such as streptococcal protein G; and transferrin. The substance regulates the solubility of the peptide; improves the stability of the peptide such as protease resistance or delivers the peptide to a specific tissue or organ.

In the present invention, delivery of a peptide into the brain can be accomplished by several methods such as, inter alia, neurosurgical implants, blood-brain barrier disruption, lipid mediated transport, carrier mediated influx or efflux, plasma protein-mediated transport, receptor-mediated transcytosis, absorptive-mediated transcytosis, neuropeptide transport at the blood-brain barrier, and genetically engineering “Trojan horses” for drug targeting. The above methods are performed for example as described in “Brain Drug Targeting: the future of brain drug development”, W. M. Pardridge, Cambridge University Press, Cambridge, UK (2001).

A known technique for allowing peptide and other products to cross the blood-brain barrier is to bind a well-known class of relatively short peptides. These well-known peptides are also known as plasma membrane transducing peptides, protein transducing domains, brain shuttles or cell-permeable peptides, and can have, for example, 5 to 30 amino acids. Such peptides typically have a cationic charge from an above normal representation (relative to proteins in general) of arginine and/or lysine residues that is believed to facilitate their passage across membranes. Some such peptides have at least 5, 6, 7 or 8 arginine and/or lysine residues. Examples include the antennapedia protein (Bonfanti, Cancer Res. 57, 1442-6 (1997)) (and variants thereof), the tat protein of human immunodeficiency virus, the protein VP22, the product of the UL49 gene of herpes simplex virus type 1, Penetratin, SynB1 and 3, Transportan, Amphipathic, gp41NLS, polyArg, and several plant and bacterial protein toxins, such as ricin, abrin, modeccin, diphtheria toxin, cholera toxin, anthrax toxin, heat labile toxins, and Pseudomonas aeruginosa exotoxin A (ETA). Other examples are described in the following references (Temsamani, Drug Discovery Today, 9 (23): 1012-1019, 2004; De Coupade, Biochem J., 390:407-418, 2005; Saalik Bioconjugate Chem. 15:1246-1253, 2004; Zhao, Medicinal Research Reviews 24 (1): 1-12, 2004; Deshayes, Cellular and Molecular Life Sciences 62:1839-49, 2005); Gao, ACS Chem. Biol. 2011, 6, 484-491, SG3), Stalmans PLOS ONE 2013, 8 (8) c71752, 1-11 and supplement; Figueiredo et a I., IUBMB Life 66, 182-194 (2014); Copolovici et a L, ACS Nano, 8, 1972-94 (2014); Lukanowski, Biotech J. 8, 918-930 (2013); Stockwell, Chem. Biol. Drug Des. 83, 507-520 (2014); Stanzl et a L, Accounts. Chem. Res/46, 2944-2954 (2013); Oiler-Salvia et a L, Chemical Society Reviews 45:10.1039/c6cs00076b (2016); Behzad Jafari et al., (2019) Expert Opinion on Drug Delivery, 16:6, 583-605 (2019) (all incorporated by reference). Still other strategies use additional methods or compositions to enhance delivery of cargo molecules such as the PSD-95 inhibitors to the brain (Dong, Theranostics 8 (6): 1481-1493 (2018).

In one embodiment, the present application includes a prophylactic and/or therapeutic agent for the prevention and/or treatment of opioid tolerance or opioid dependence comprising the peptide which inhibits the dimer formation of the opioid receptor, wherein the dimer is a heterodimer formed from two opioid receptors selected from the group consisting of the μ type (MOR), the κ type (KOR), and the δ type (DOR), wherein the dimer is a MOR and DOR heterodimer or a KOR and DOR heterodimer.

The peptide preferably comprises any one amino acid sequence selected from the group consisting of the amino acid sequences of Sequence ID Numbers 1 to 30 and the amino acid sequences of Sequence ID Numbers 41 to 67 listed in Table B. The peptide more preferably comprises any one amino acid sequence selected from the group consisting of the amino acid sequences of Sequence ID Numbers 2 to 8, 11 to 13, 15, 17 to 30 and the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 1 to 30 and in the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 2 to 8, 11 to 13, 15, 17 to 30 and in the amino acid sequences of Sequence ID Numbers 41 to 67 listed in Table B. The peptide may comprise any one amino acid sequence having at least 80%, 90%, 95%, or 99% amino acid sequence identity with any of these amino acid sequences.

In one embodiment, the present application includes a prophylactic and/or therapeutic agent for the prevention and/or treatment of opioid tolerance or opioid dependence comprising the peptide which inhibits the dimer formation of the opioid receptor, wherein the dimer is a homodimer of MOR, KOR or DOR.

The peptide preferably comprises any one amino acid sequence selected from the group consisting of the amino acid sequences of Sequence ID Numbers 31 to 40, amino acid sequences having one or more conservative amino acid substitutions in the amino acid sequences of Sequence ID Numbers 31 to 40 or amino acid sequences having at least 80%, 90%, 95%, or 99% amino acid sequence identity with any of these amino acid sequences.

Examples of forms of the prophylactic and/or therapeutic agent for the prevention and/or treatment of opioid tolerance or opioid dependence include injectables (including intravenous preparations and lyophilized preparations), sublingual preparations, nasal absorbents, transdermal absorbents, capsules, tablets, suppositories, ointments, granules, aerosols, rounds, dispersions, suspensions, emulsions, and bioimplantable preparations.

The dosage of a preparation containing the peptide of the invention is not limited to a pharmacologically effective amount and can be determined according to the species of the individual, type of disease, symptoms, sex, age, pre-existing disease, and other factors, but is usually 0.01 to 1000 mg/kg, preferably 0.1 to 100 mg/kg. The dose can be administered once a day, twice a day, or three or more times a day.

(Agent that Enhance Opioid Analgesia)

In one embodiment, the present application includes an agent that enhances opioid analgesia comprising the peptide which inhibits the dimer formation of the opioid receptor, wherein the dimer is a heterodimer or a homodimer formed from one or two opioid receptors selected from the group consisting of the μ type (MOR), the κ type (KOR), and the 8 type (DOR). The above dimer is preferably a heterodimer, and it is more preferred to be a heterodimer of MOR and DOR. A morphine-induced analgesia can be enhanced by the continuous administration of the peptide which inhibits the dimer formation of the opioid receptor. The peptide which inhibits the dimer formation of the opioid receptor can be used as a potentiator of opioid analgesia.

The use of the agent that enhances opioid analgesia can reduce the dose of opioids taken. This can prevent, ameliorate, or deter the development of opioid dependence and opioid tolerance. In one embodiment, the dose of the opioid may be reduced by 10%, 20%, 30%, 50%, or 80% by weight as compared to the standard amount administered to a patient.

The agent that enhances opioid analgesia of the present invention is used in combination with opioids. The agent that enhances opioid analgesia of the invention may be administered before, at the same time as, or after opioids are administered.

The agent that enhances opioid analgesia may be administered to subjects who need to use opioids, who use opioids, who plan to use opioids, who are at risk of acquiring opioid tolerance, who have opioid tolerance, who are at risk of opioid dependence, or who are opioid dependent.

The description of the peptides, related matters and other in the previous section regarding prophylactic and/or therapeutic agent for the prevention and/or treatment of opioid tolerance or opioid dependence is directly applicable.

### (Combination Drug of Opioids and the Peptide of the Present Invention)

The present invention includes a combination drug of opioids and the peptide of the present invention. This combination drug may be a pharmaceutical composition comprising the peptide of the present invention and an opioid. The pharmaceutical composition may be for sublingual administration.

The combination drug of the present invention can prevent and/or treat opioid tolerance and opioid dependence in addition to the analgesic effect obtained with opioids alone, and can also enhance the effect of opioids. The use of the combination drug of opioids and the peptide of the present invention can reduce the dose of opioids taken. This can prevent, ameliorate, or deter the development of opioid dependence and opioid tolerance. In one embodiment, the dose of the opioid may be reduced by 10%, 20%, 30%, 50%, or 80% by weight as compared to the standard amount administered to a patient.

The peptide of the present invention may be administered before, at the same time as, or after opioids are administered in the combination drug of the present invention.

The combination drug of the present invention may be administered to the subjects suffering from pain as well as the subjects who are at risk of acquiring opioid tolerance, who have opioid tolerance, who are at risk of opioid dependence, or who are opioid dependent.

(Method of Prevention and/or Treatment of Opioid Tolerance or Opioid Dependence)

In one embodiment, the present application includes a method of prevention and/or treatment of opioid tolerance or opioid dependence comprising administering a peptide which inhibits the formation of heterodimers or homodimers formed from one or two types of opioid receptor selected from the group consisting of the μ-type (MOR), the κ-type (KOR) and the δ-type (DOR) to a subject.

The peptide preferably comprises any one amino acid sequence selected from the group consisting of the amino acid sequences of Sequence ID Numbers 1 to 40 and the amino acid sequences of Sequence ID Numbers 41 to 67 listed in Table B. The peptide more preferably comprises any one amino acid sequence selected from the group consisting of the amino acid sequences of Sequence ID Numbers 2 to 8, 11 to 13, 15, 17 to 40 and the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 1 to 40 and in the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 2 to 8, 11 to 13, 15 and 17 to 40 and in the amino acid sequences of Sequence ID Numbers 41 to 67 listed in Table B. The peptide may comprise any one amino acid sequence having at least 80%, 90%, 95%, or 99% amino acid sequence identity with any of these amino acid sequences.

In one embodiment, the present application includes a method of prevention and/or treatment of opioid tolerance or opioid dependence comprising administering a peptide which inhibits the formation of heterodimers or homodimers formed from one or two types of opioid receptor selected from the group consisting of the μ-type (MOR), the κ-type (KOR) and the δ-type (DOR) to a subject, wherein the peptide inhibits the formation of MOR and DOR or KOR and DOR heterodimers.

The peptide preferably comprises any one amino acid sequence selected from the group consisting of the amino acid sequences of Sequence ID Numbers 1 to 30 and the amino acid sequences of Sequence ID Numbers 41 to 67 listed in Table B. The peptide more preferably comprises any one amino acid sequence selected from the group consisting of the amino acid sequences of Sequence ID Numbers 2 to 8, 11 to 13, 15, 17 to 30 and the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 1 to 30 and in the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 2 to 8, 11 to 13, 15, 17 to 30 and in the amino acid sequences of Sequence ID Numbers 41 to 67 listed in Table B. The peptide may comprise any one amino acid sequence having at least 80%, 90%, 95%, or 99% amino acid sequence identity with any of these amino acid sequences.

In one embodiment, the present application includes a method of prevention and/or treatment of opioid tolerance or opioid dependence comprising administering a peptide which inhibits the formation of heterodimers or homodimers formed from one or two types of opioid receptor selected from the group consisting of the μ-type (MOR), the κ-type (KOR) and the δ-type (DOR) to a subject, wherein the peptide inhibits the formation of homodimers of MOR, KOR or DOR.

The peptide preferably comprises any one amino acid sequence selected from the group consisting of the amino acid sequences of Sequence ID Numbers 31 to 40, amino acid sequences having one or more conservative amino acid substitutions in the amino acid sequences of Sequence ID Numbers 31 to 40 and amino acid sequences having at least 80%, 90%, 95%, or 99% amino acid sequence identity with any of these amino acid sequences.

### (Method of Enhancing Analgesic Action of Opioids)

In one embodiment, the present application includes a method of enhancing analgesic action of opioids comprising administering a peptide which inhibits the formation of heterodimers or homodimers formed from one or two types of opioid receptor selected from the group consisting of the μ-type (MOR), the κ-type (KOR) and the δ-type (DOR) of the opioid receptor to a subject.

The method of enhancing opioid analgesia can reduce the dose of opioids taken. This can prevent, ameliorate, or deter the development of opioid dependence and opioid tolerance. In one embodiment, the dose of the opioid may be reduced by 10%, 20%, 30%, 50%, or 80% by weight as compared to the standard amount administered to a patient.

The peptide which inhibits the formation of heterodimers or homodimers formed from one or two types of opioid receptor selected from the group consisting of the μ-type (MOR), the κ-type (KOR) and the δ-type (DOR) of the opioid receptor may be administered before, at the same time as, or after opioids are administered in the method of enhancing analgesic action of opioids.

The peptide which inhibits the formation of heterodimers or homodimers formed from one or two types of opioid receptor selected from the group consisting of the μ-type (MOR), the κ-type (KOR) and the δ-type (DOR) of the opioid receptor may be administered to subjects who need to use opioids, who use opioids, who plan to use opioids, who are at risk of acquiring opioid tolerance, who have opioid tolerance, who are at risk of opioid dependence, or who are opioid dependent in the method of enhancing analgesic action of opioids.

The description of the peptides in the previous section regarding method of prevention and/or treatment of opioid tolerance or opioid dependence is directly applicable.

### (Peptide)

In one embodiment, the present application includes a peptide comprising any one amino acid sequence selected from the group consisting of the amino acid sequences of Sequence ID Numbers 1 to 40 and the amino acid sequences of Sequence ID Numbers 41 to 67 listed in Table B. The peptide more preferably comprises any one amino acid sequence selected from the group consisting of the amino acid sequences of Sequence ID Numbers 2 to 8, 11 to 13, 15, 17 to 40 and the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 1 to 40 and in the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 2 to 8, 11 to 13, 15 and 17 to 40 and in the amino acid sequences of Sequence ID Numbers 41 to 67 listed in Table B. The peptide may comprise any one amino acid sequence having at least 80%, 90%, 95%, or 99% amino acid sequence identity with any of these amino acid sequences. The peptide of the present invention inhibits the formation of heterodimers or homodimers formed from one or two types of opioid receptor selected from the group consisting of the μ-type (MOR), κ-type (KOR), and δ-type (DOR) and they are effective in prevention and/or treatment of opioid tolerance or opioid dependence. In addition, the peptides of the present invention are effective in enhancing analgesic action of opioids.

In one embodiment, the present application includes a peptide preferably comprises any one amino acid sequence selected from the group consisting of the amino acid sequences of Sequence ID Numbers 1 to 30 and the amino acid sequences of Sequence ID Numbers 41 to 67 listed in Table B. The peptide more preferably comprises any one amino acid sequence selected from the group consisting of the amino acid sequences of Sequence ID Numbers 2 to 8, 11 to 13, 15, 17 to 30 and the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 1 to 30 and in the amino acid sequences of Sequence ID Numbers 41 to 67 listed in Table B. The peptide may comprise any one amino acid sequence selected from the group consisting of the amino acid sequences having one or more conservative amino acid substitutions in the amino acid sequences of Sequence ID Numbers 2 to 8, 11 to 13, 15, 17 to 30 and in the amino acid sequences of Sequence ID Numbers 41 to 67 listed in Table B. The peptide may comprise any one amino acid sequence having at least 80%, 90%, 95%, or 99% amino acid sequence identity with any of these amino acid sequences. The peptide of the present invention inhibits the formation of MOR and DOR heterodimers or KOR and DOR heterodimers.

In one embodiment, the present application includes a peptide preferably comprises any one amino acid sequence selected from the group consisting of the amino acid sequences of Sequence ID Numbers 31 to 40, amino acid sequences having one or more conservative amino acid substitutions in the amino acid sequences of Sequence ID Numbers 31 to 40 and amino acid sequences having at least 80%, 90%, 95%, or 99% amino acid sequence identity with any of these amino acid sequences. The peptide of the present invention inhibits the formation of homodimers of MOR, KOR or DOR.

### EXAMPLES

Hereinafter, the present invention will be described in more detail with reference to examples, but the present invention is not limited by these examples.

### <Example 1> Heterodimer Blockers

### DM and DK, but not MK, Heterodimers

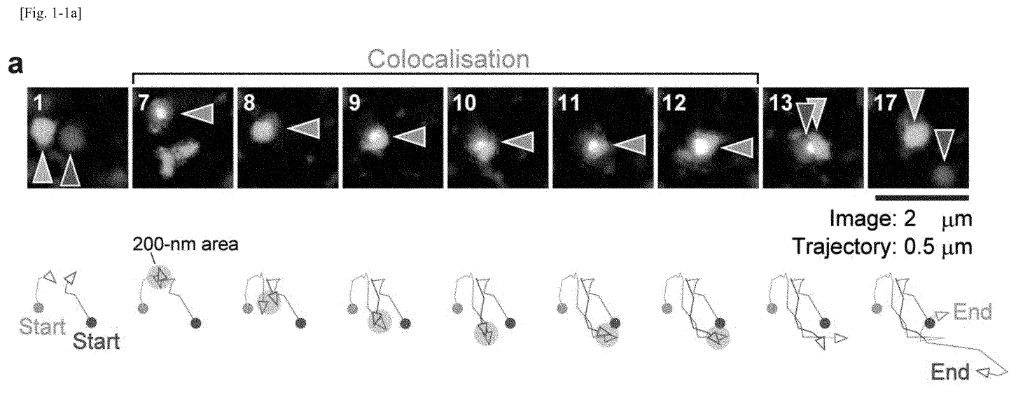

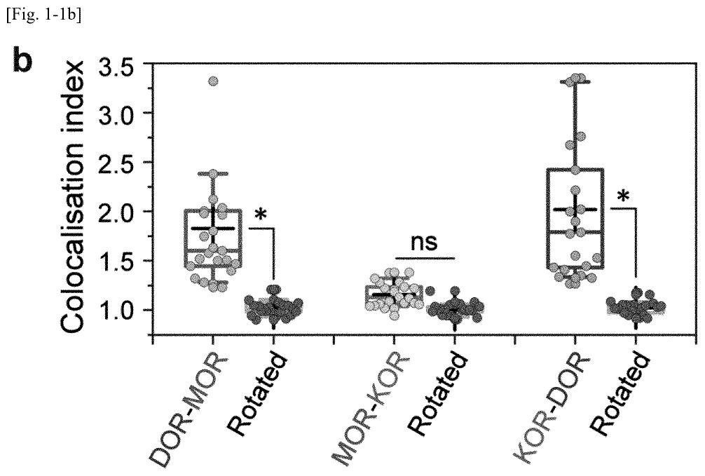

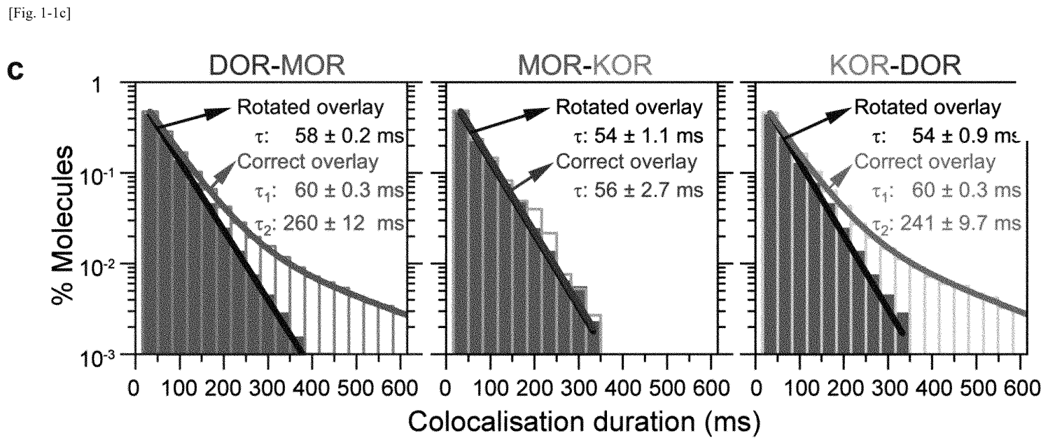

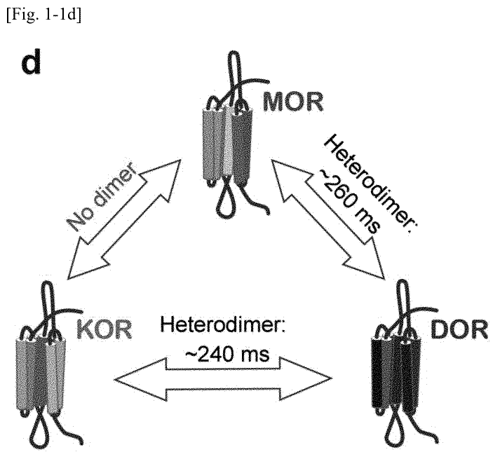

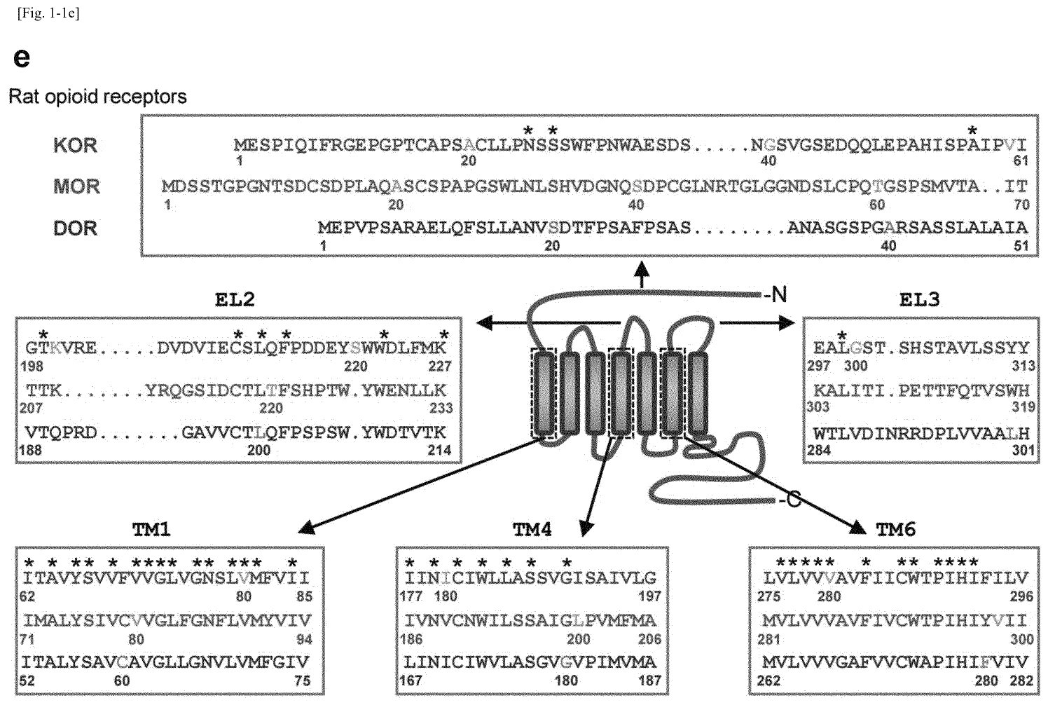

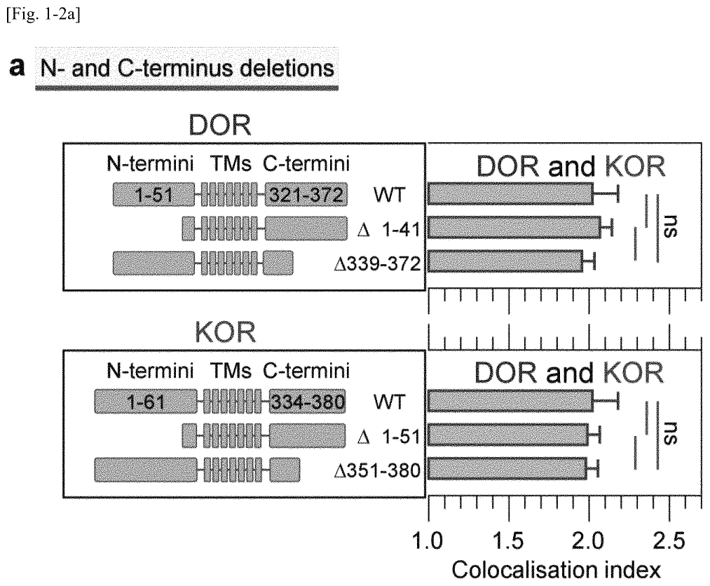

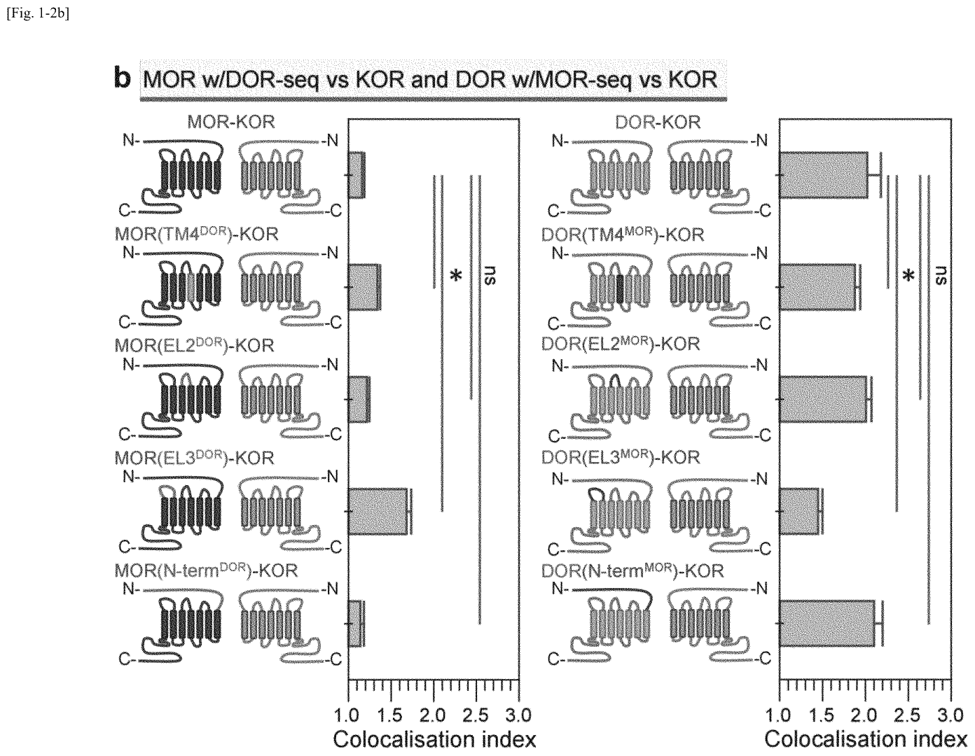

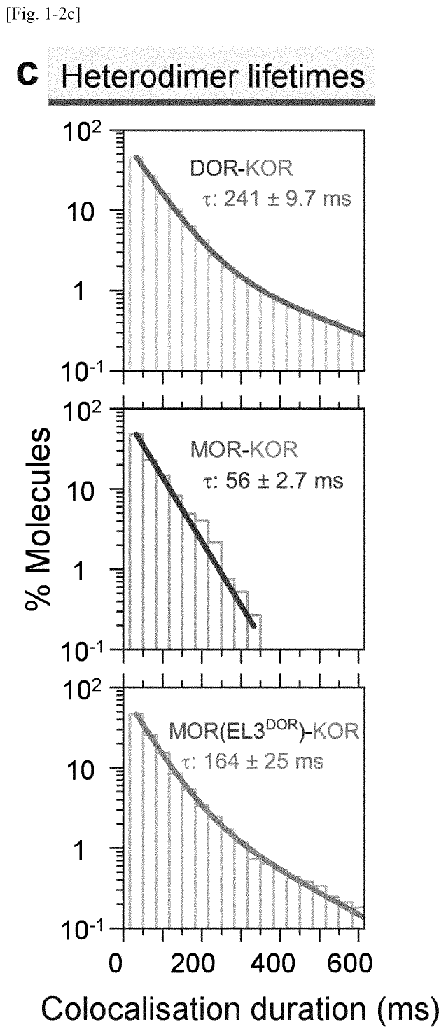

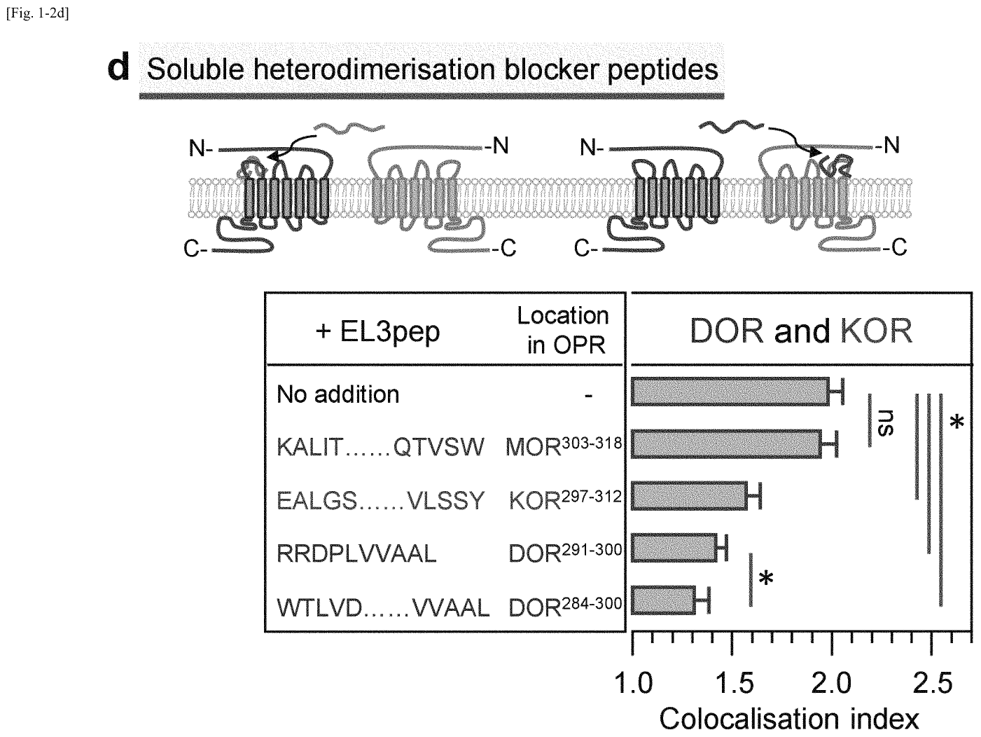

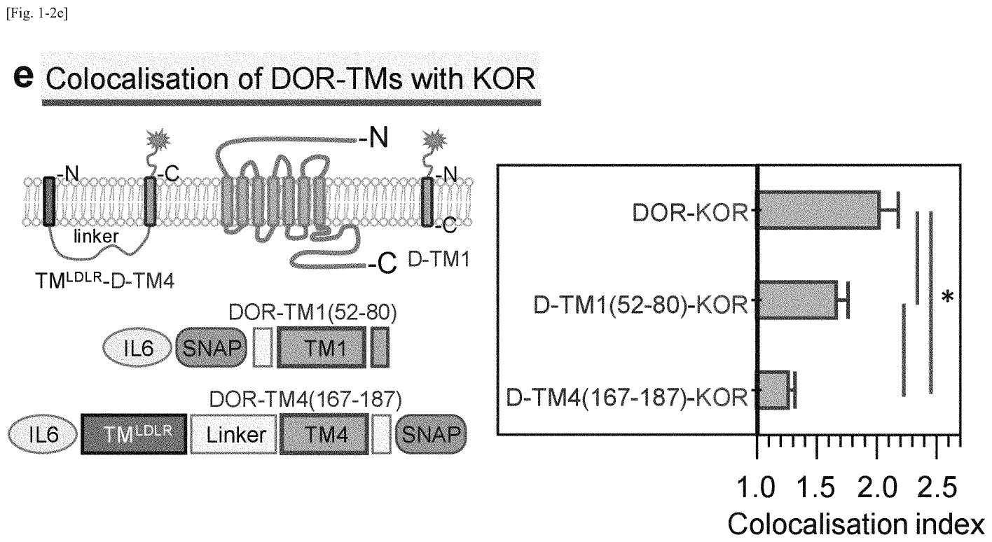

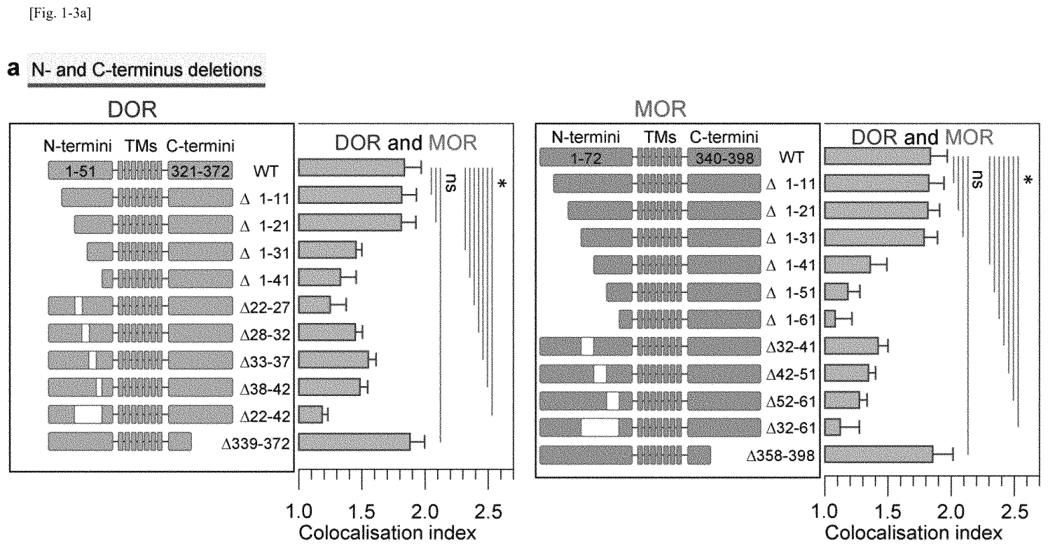

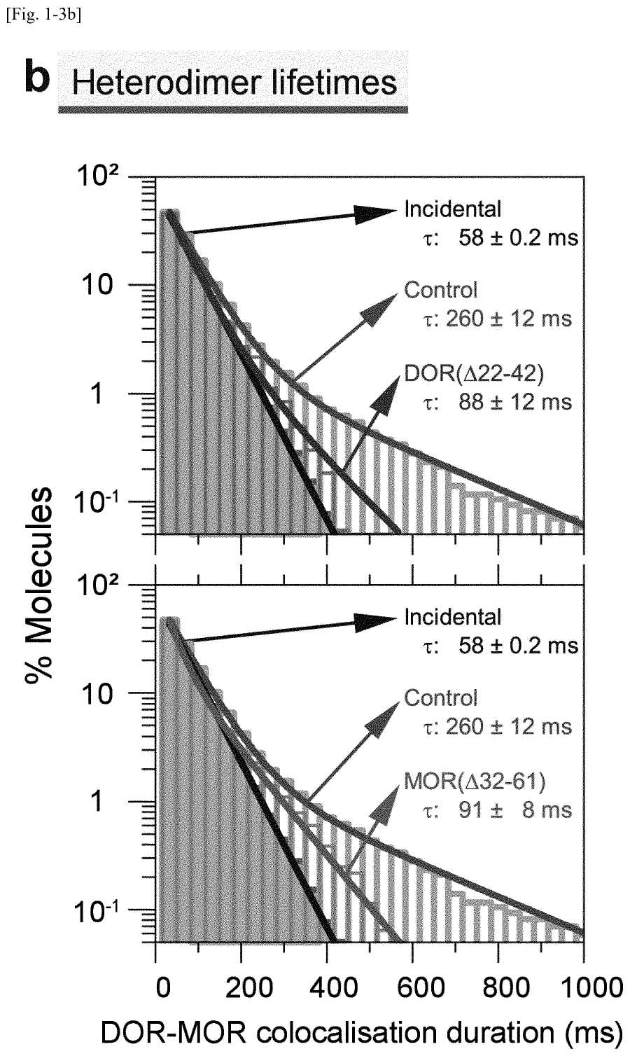

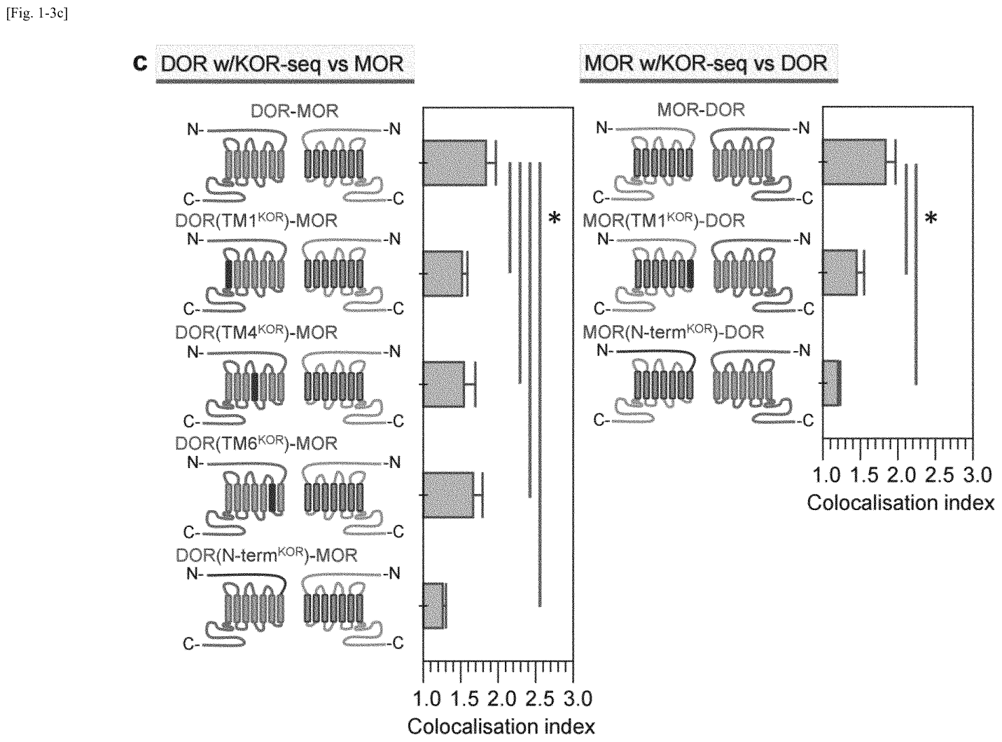

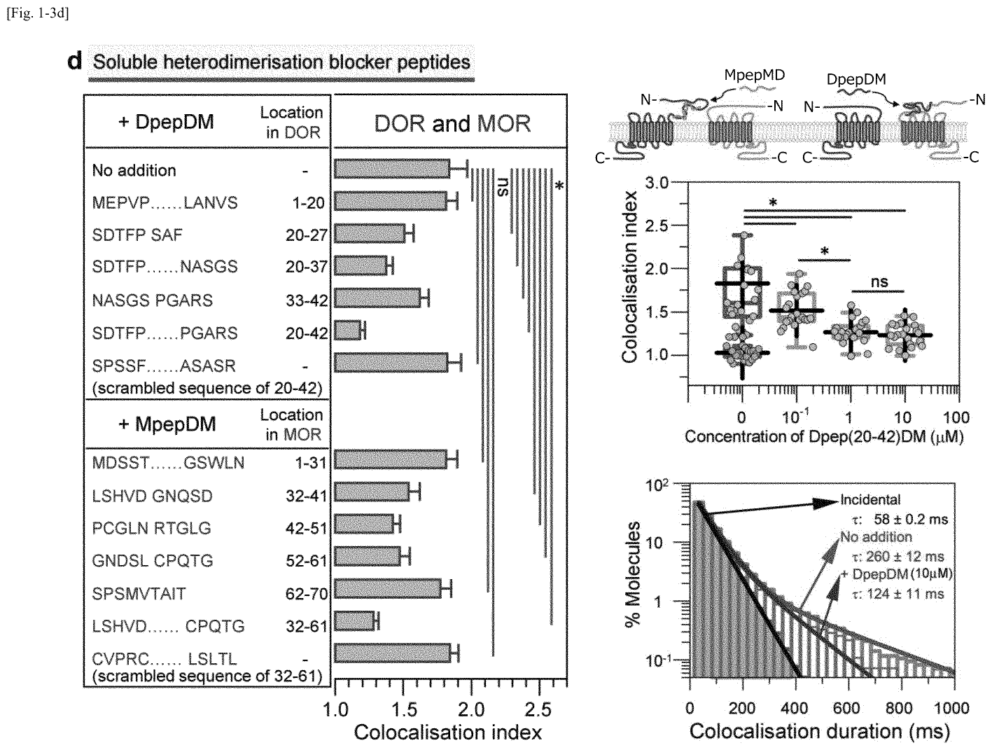

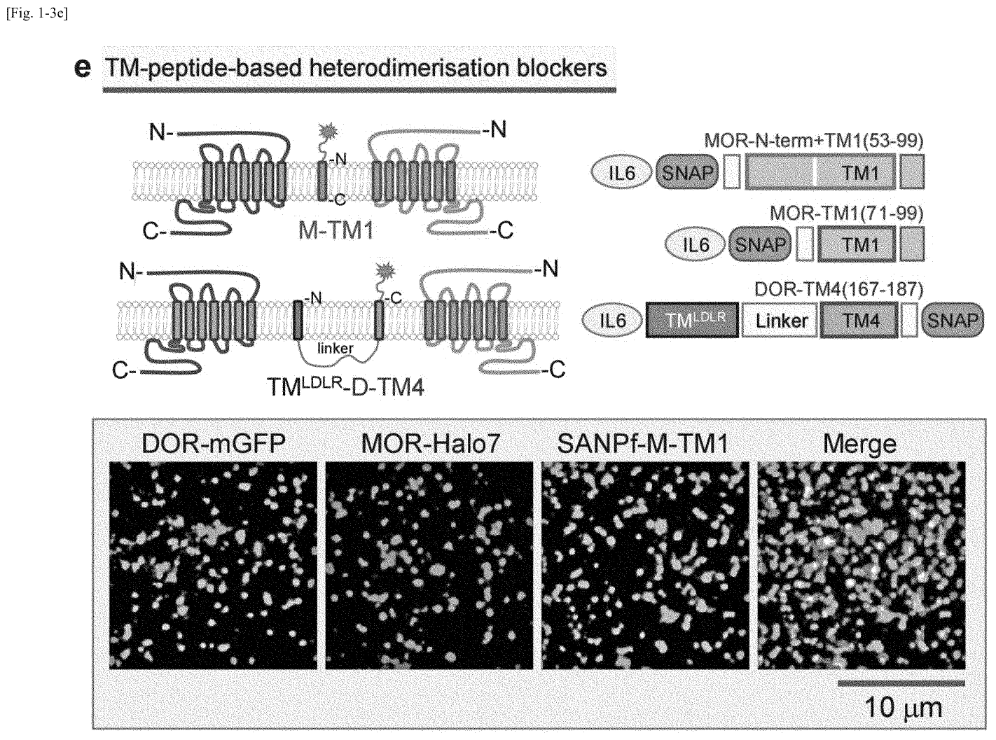

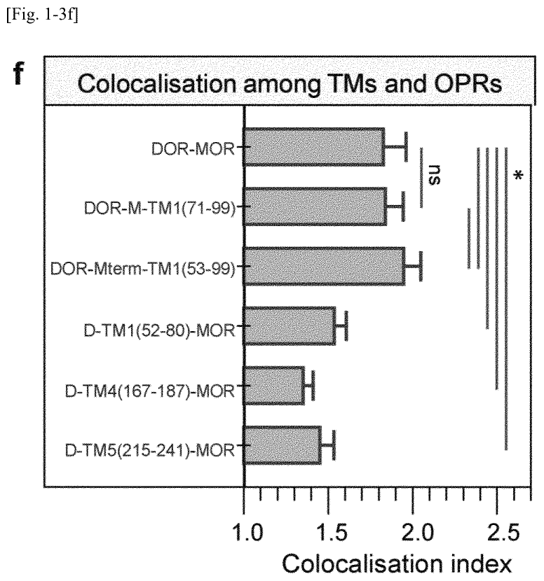

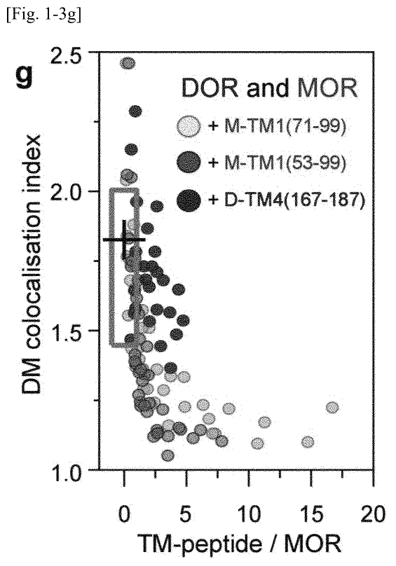

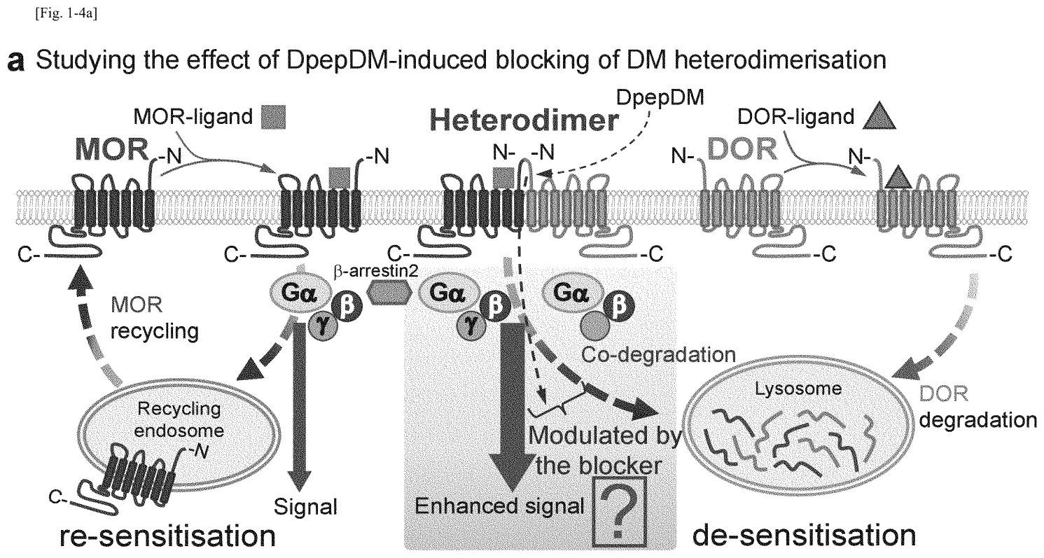

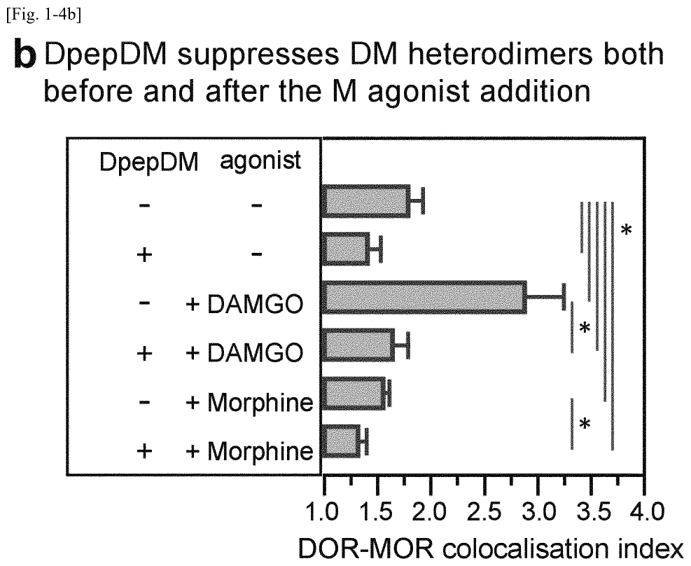

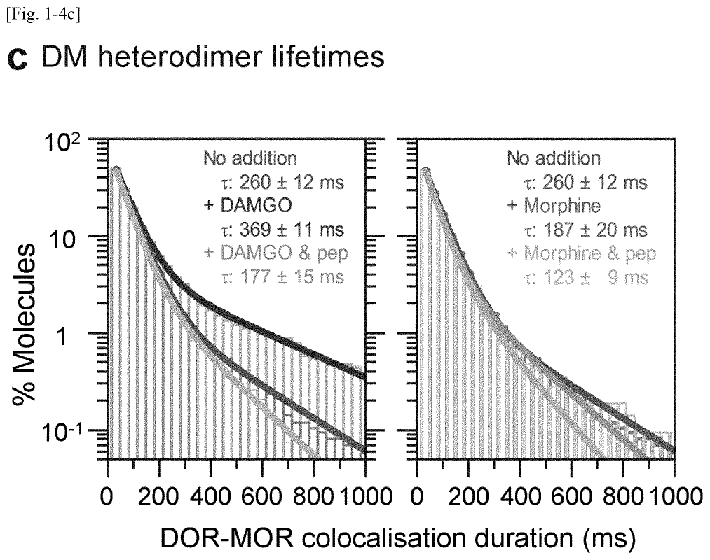

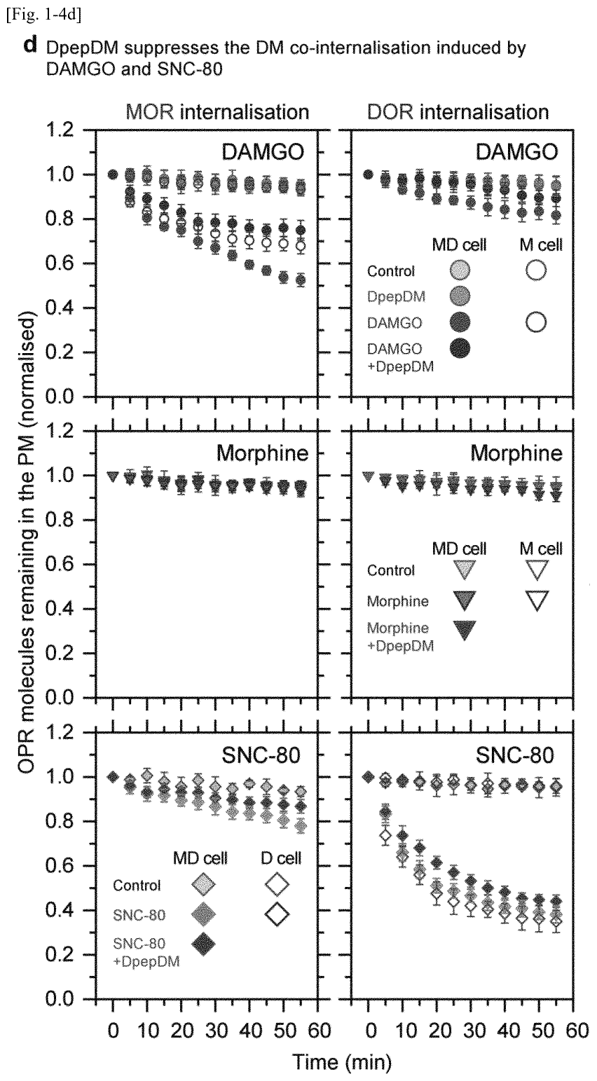

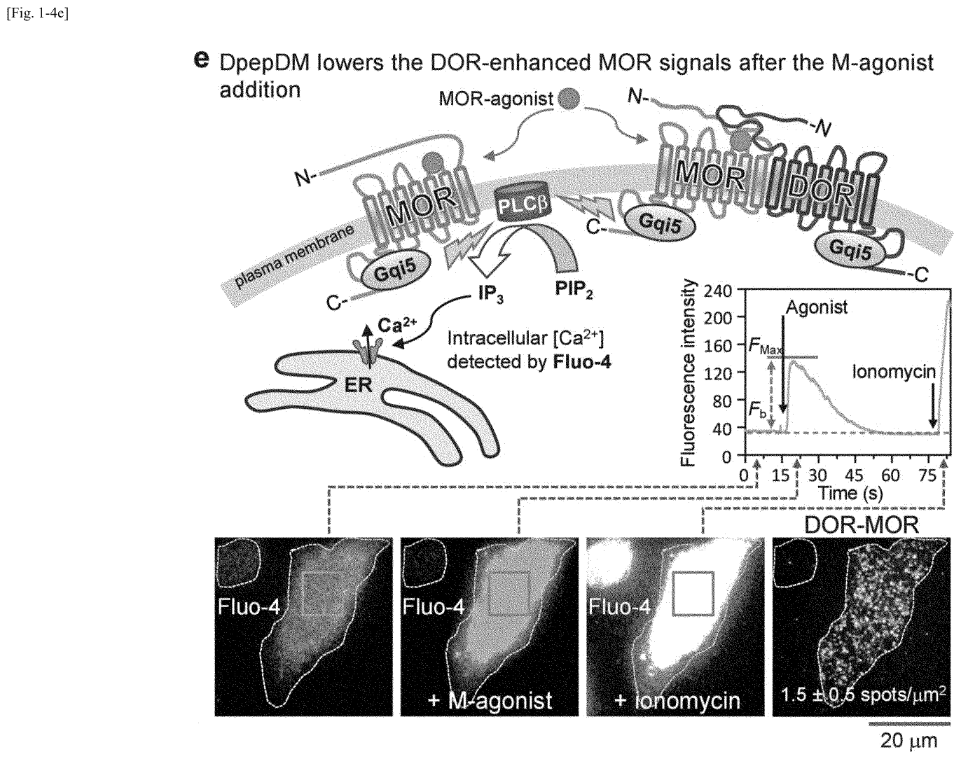

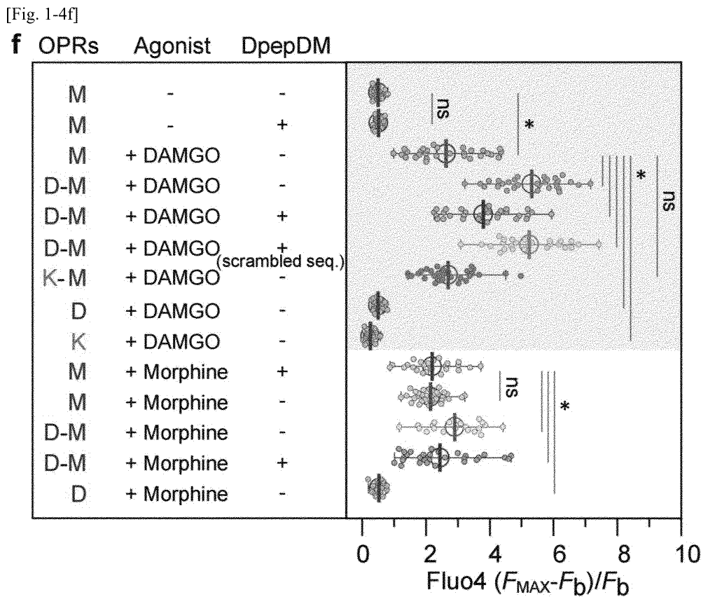

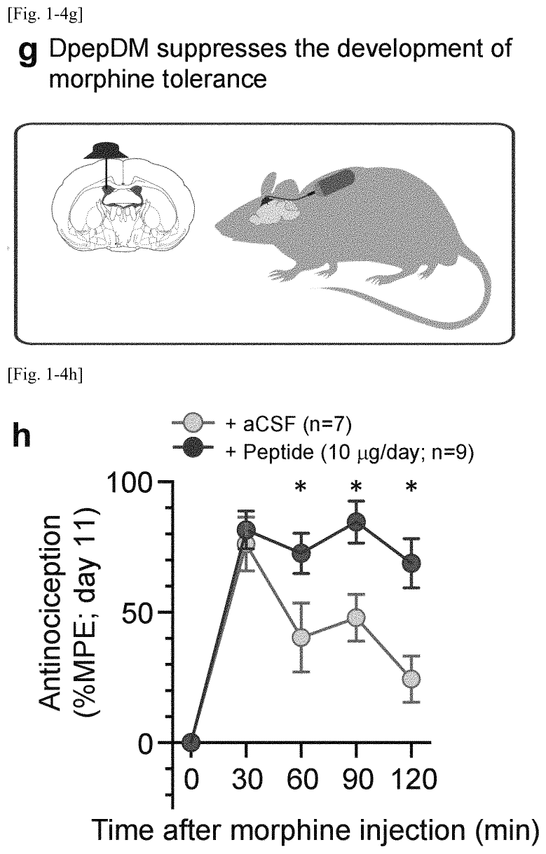

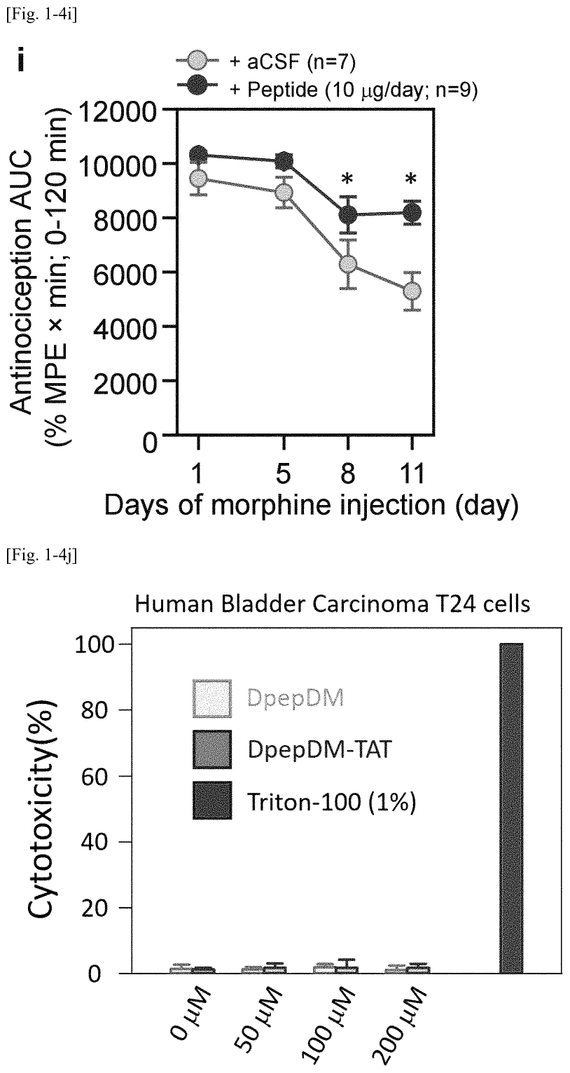

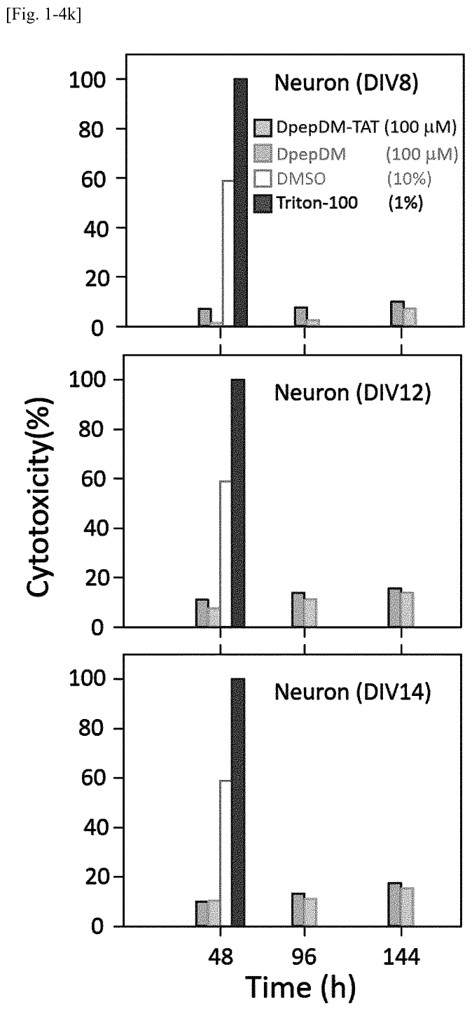

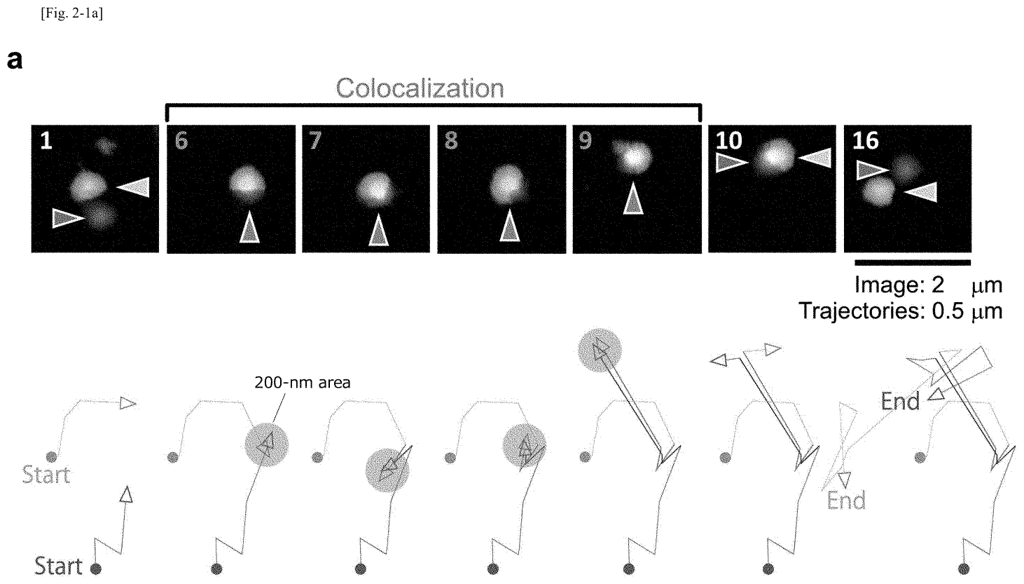

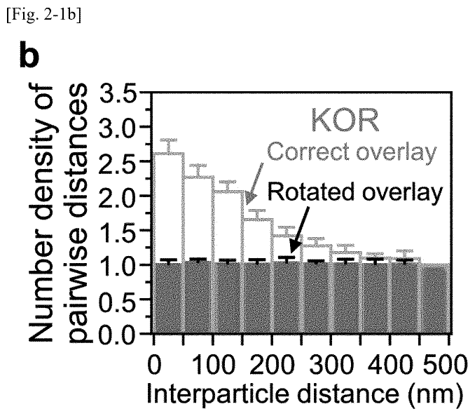

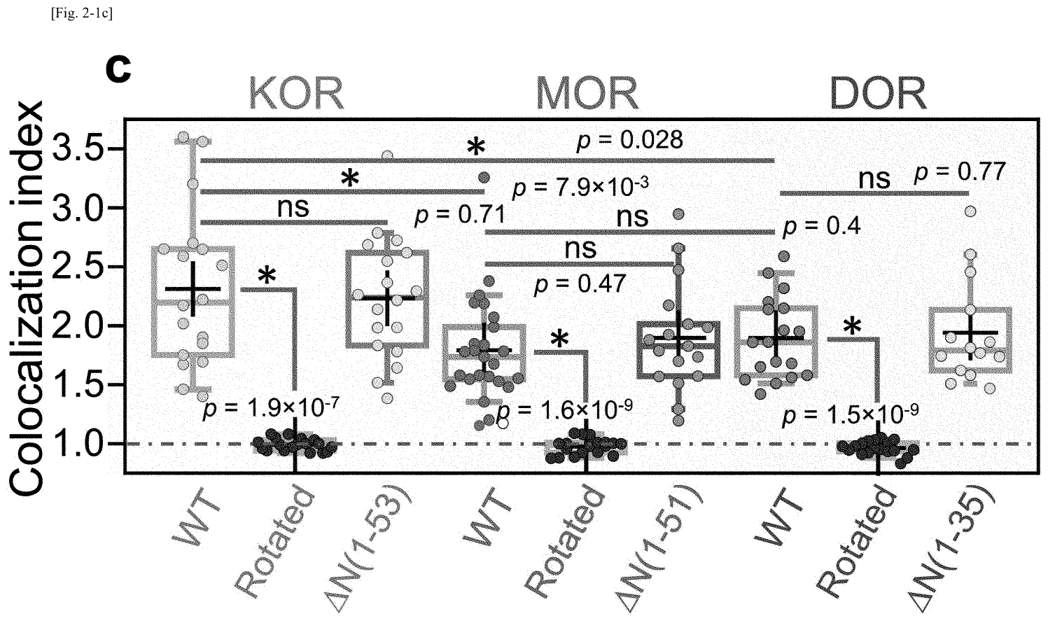

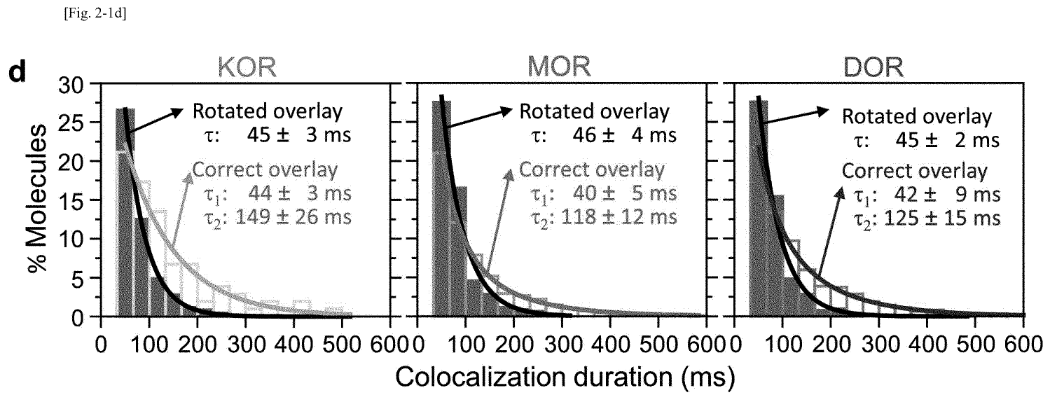

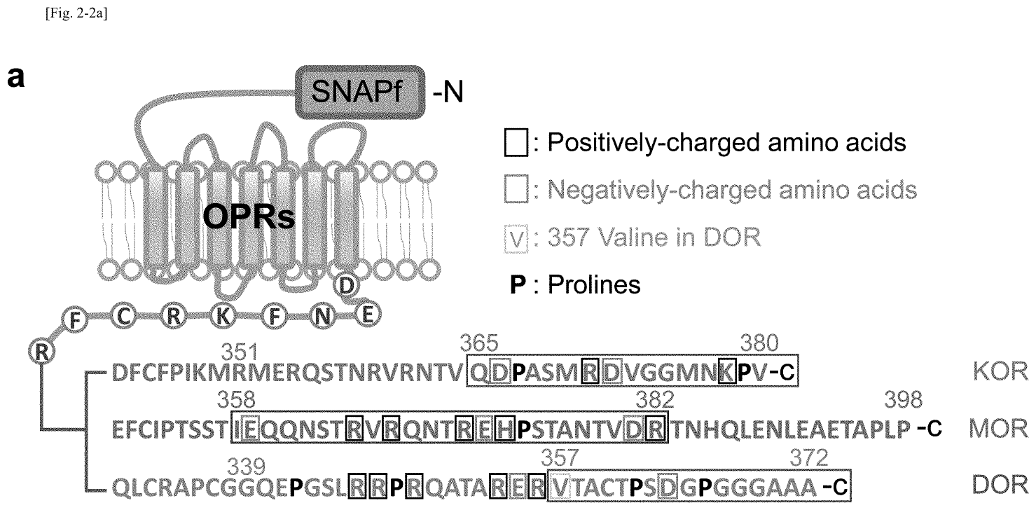

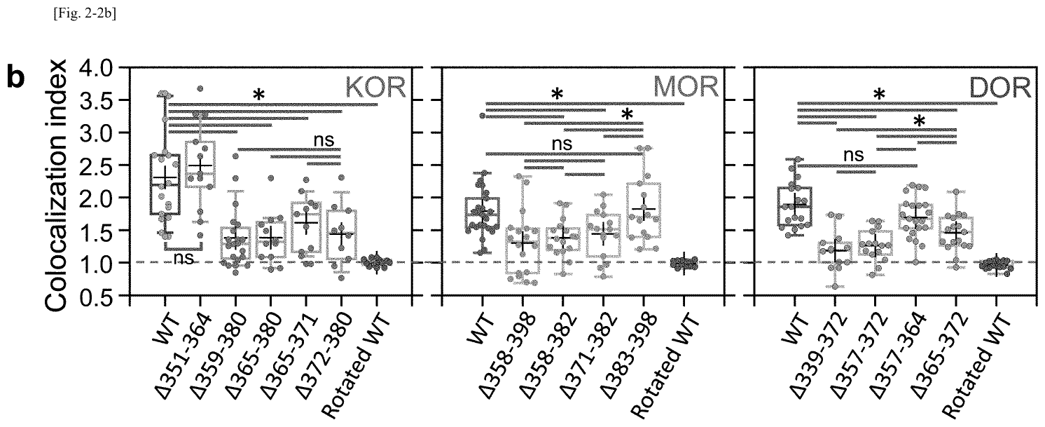

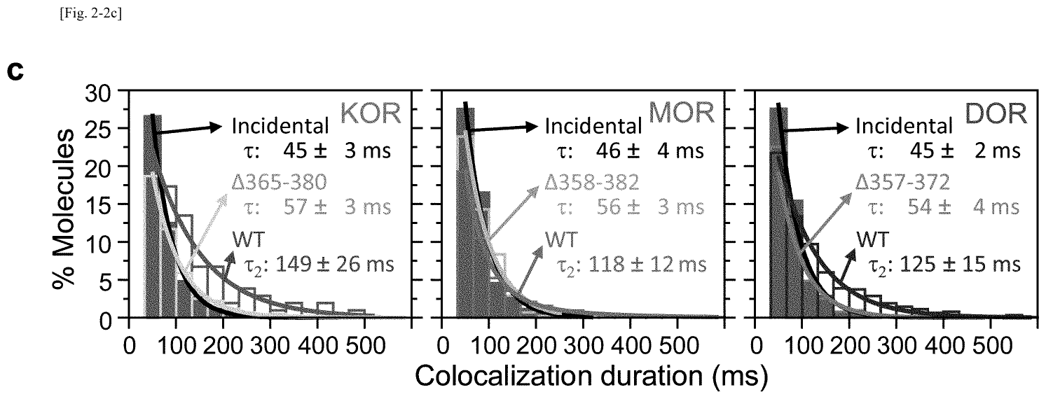

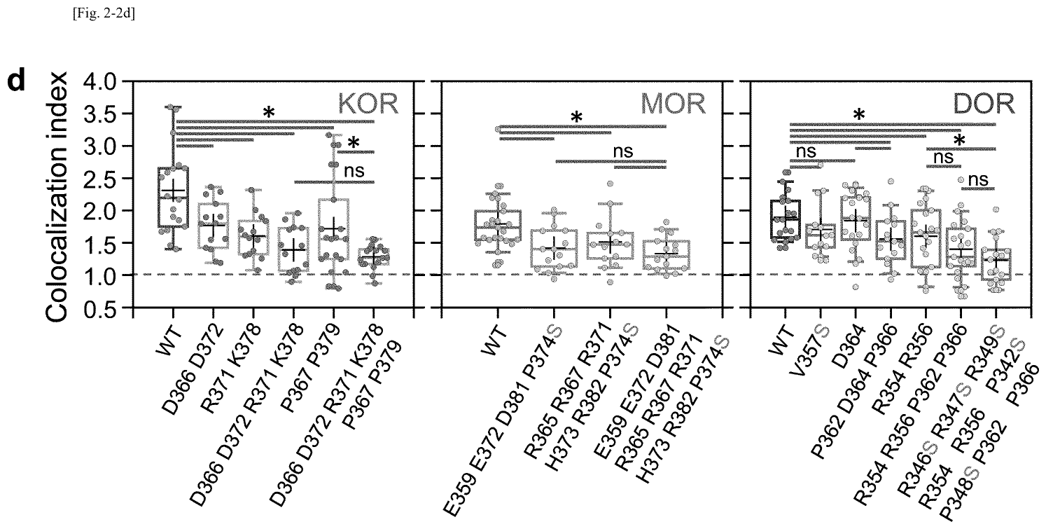

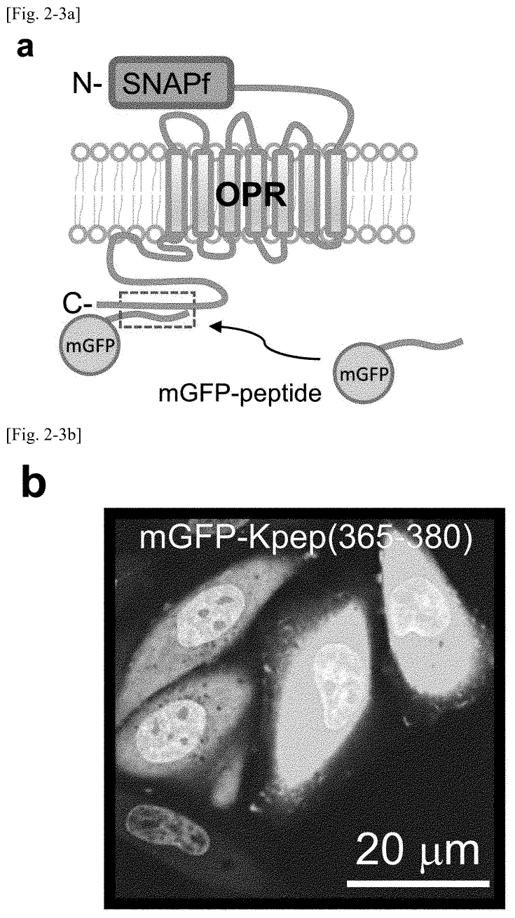

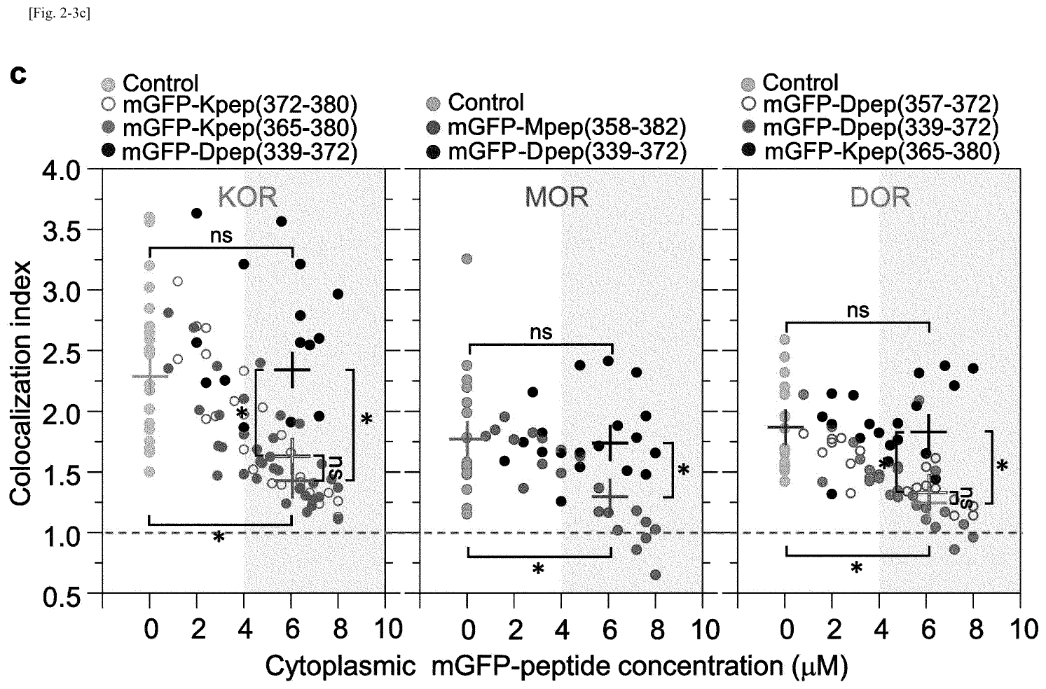

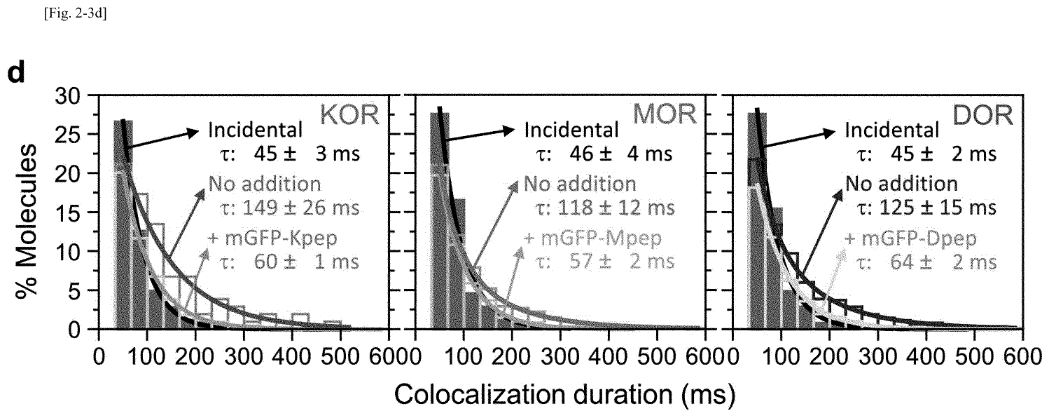

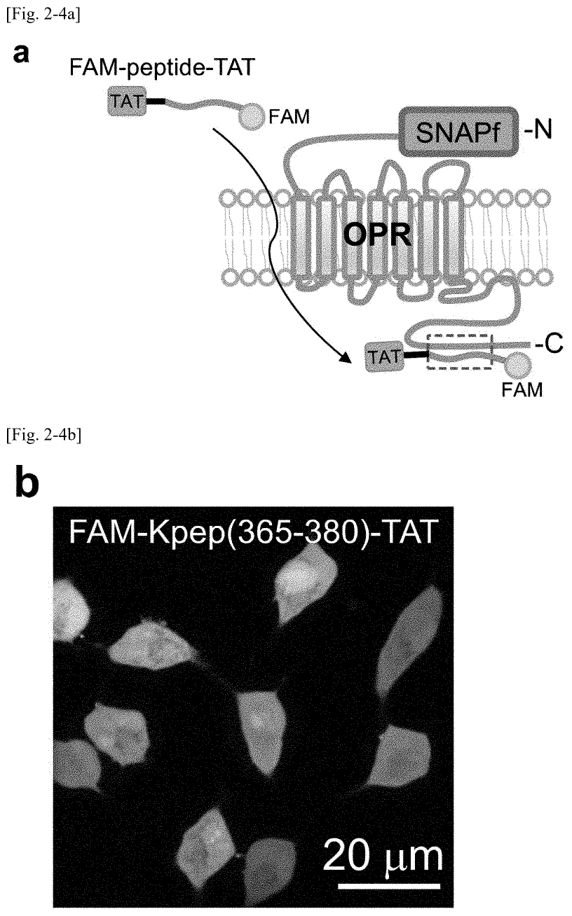

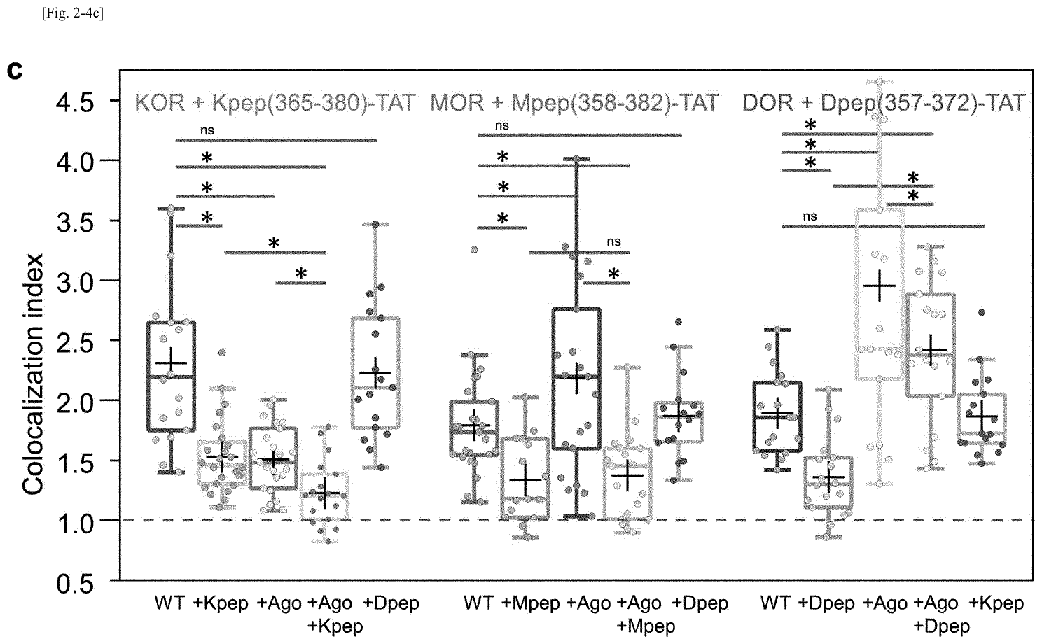

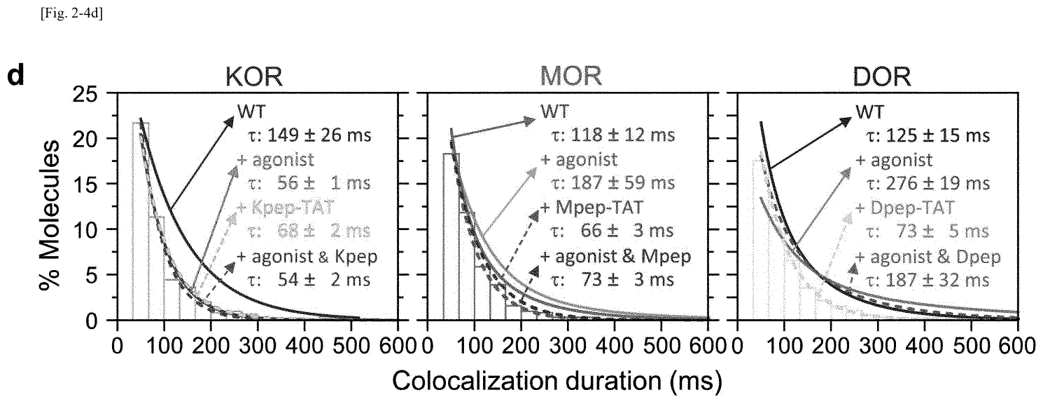

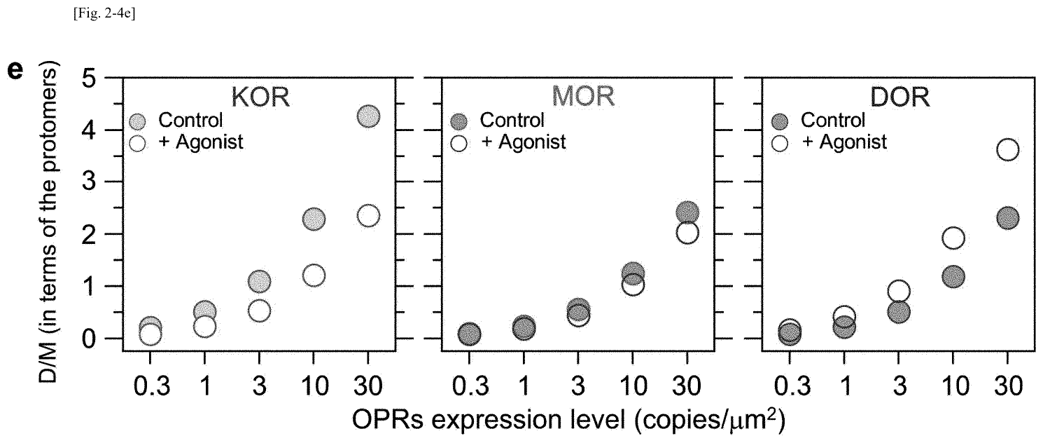

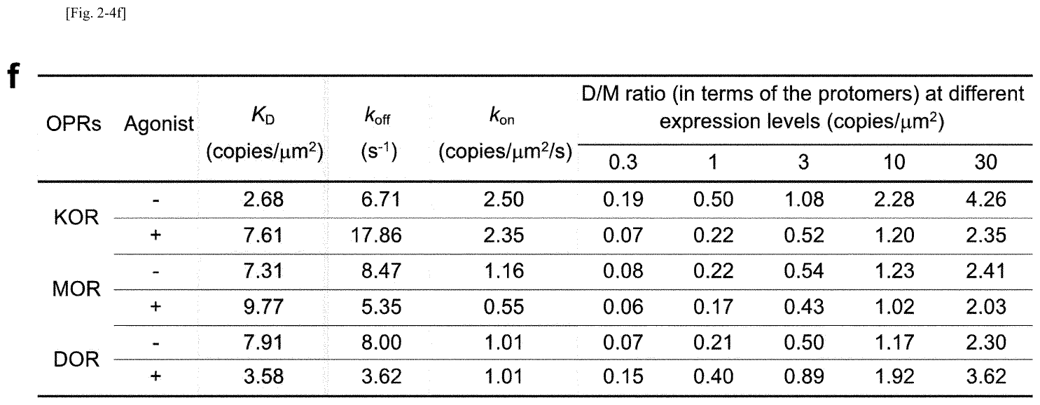

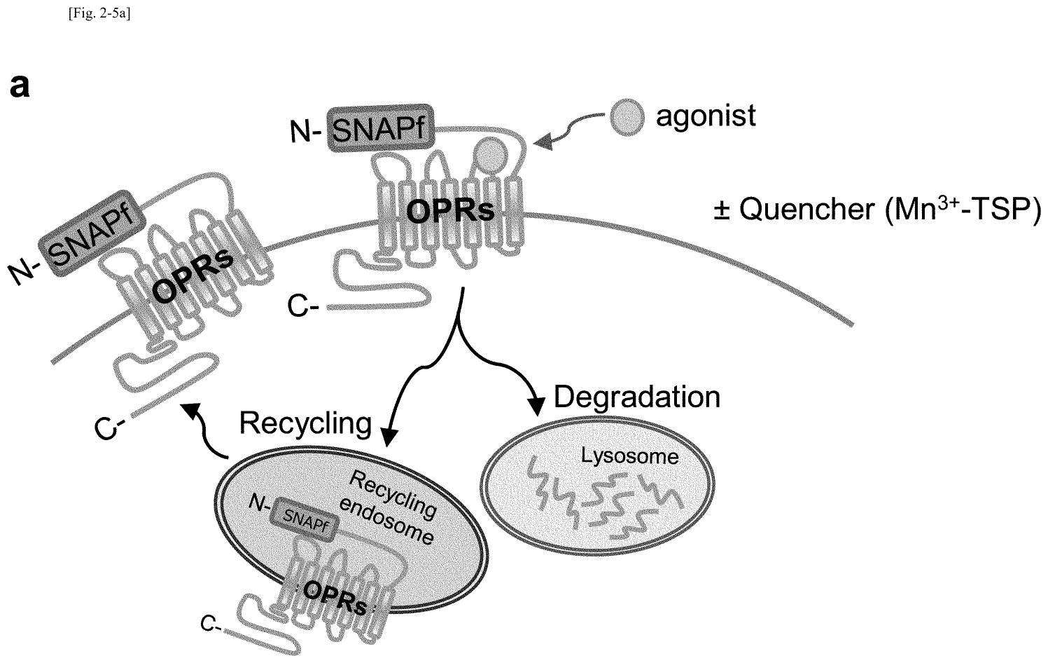

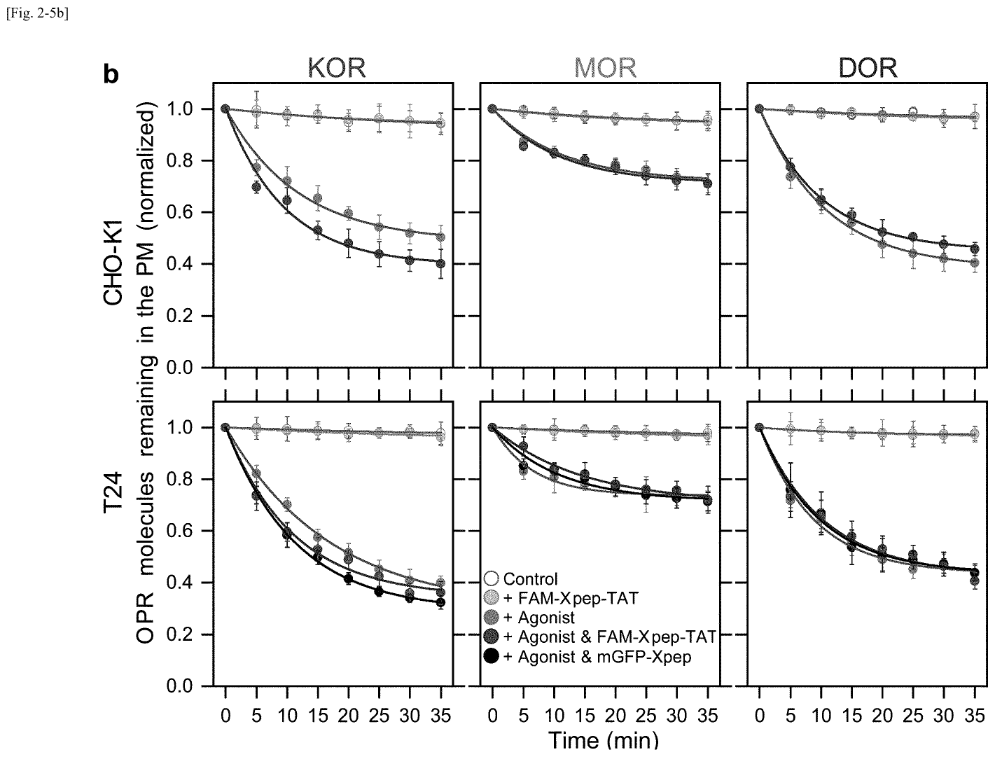

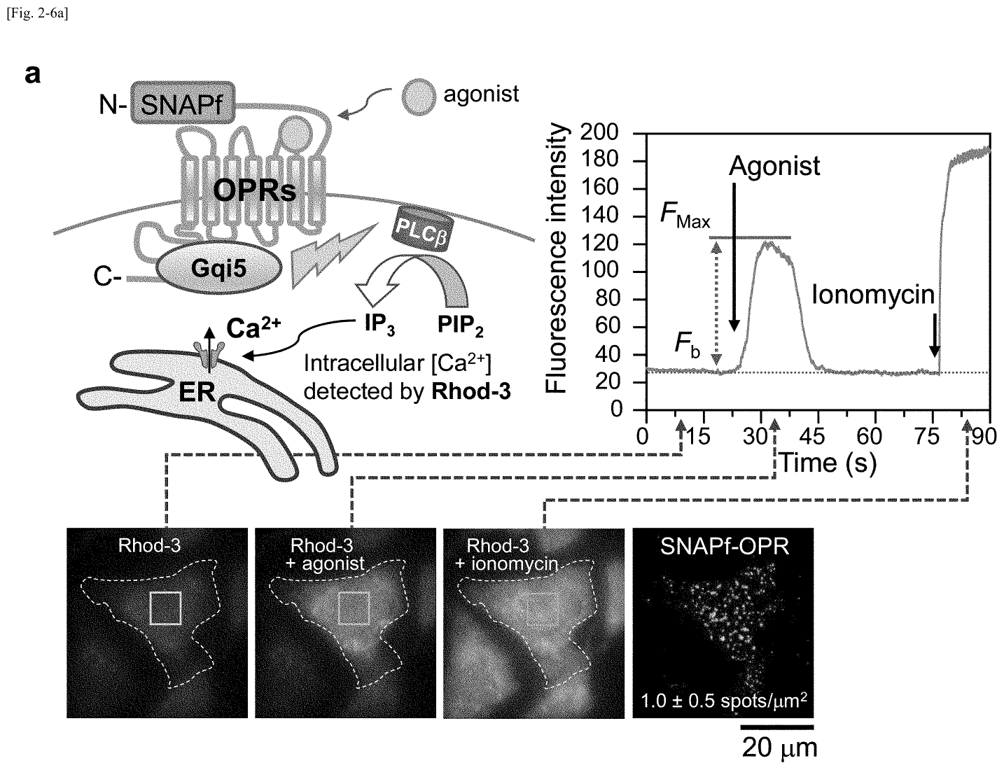

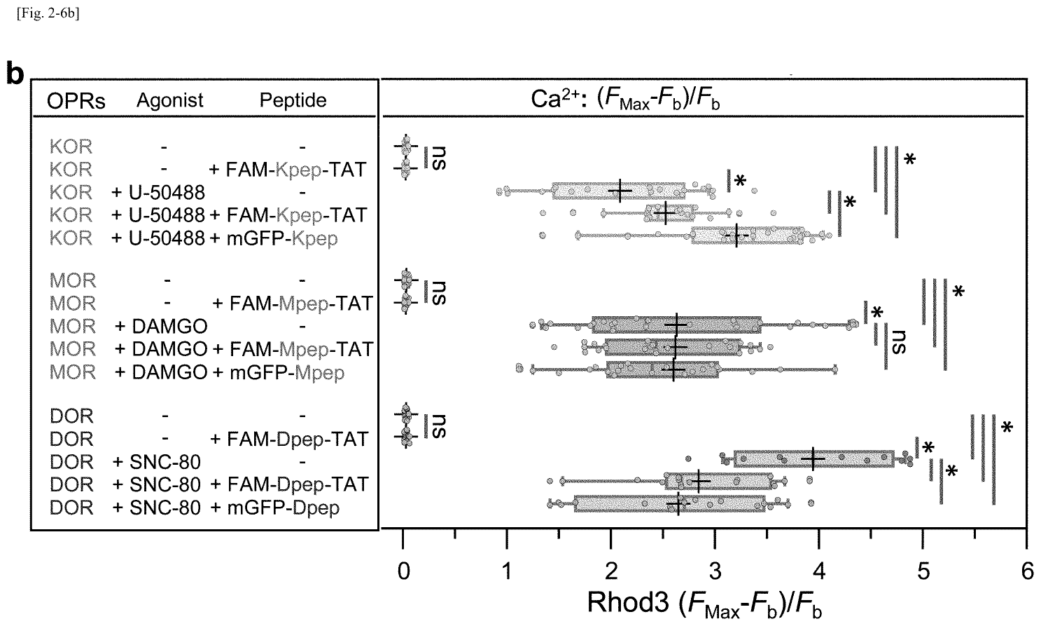

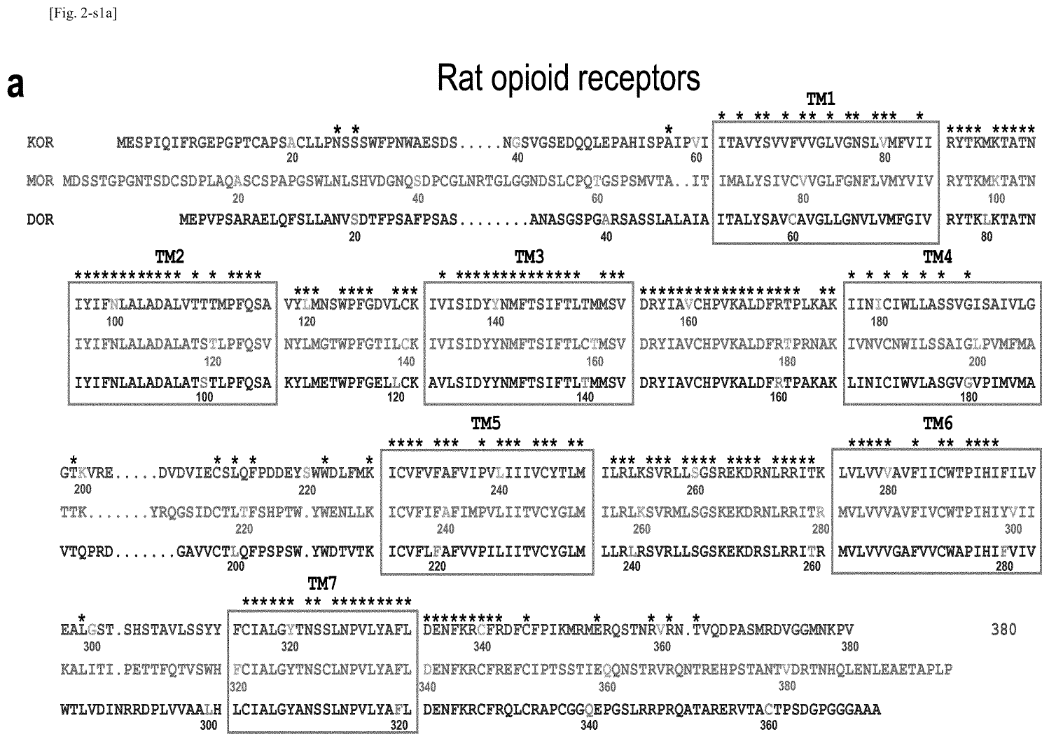

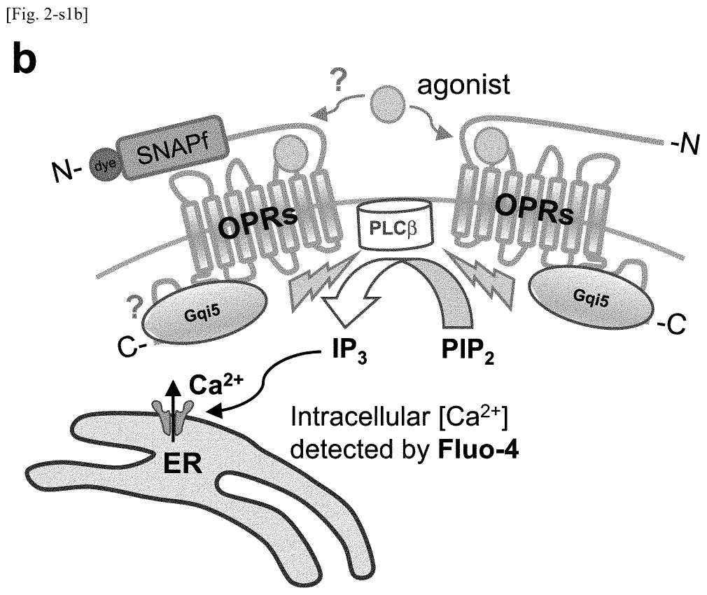

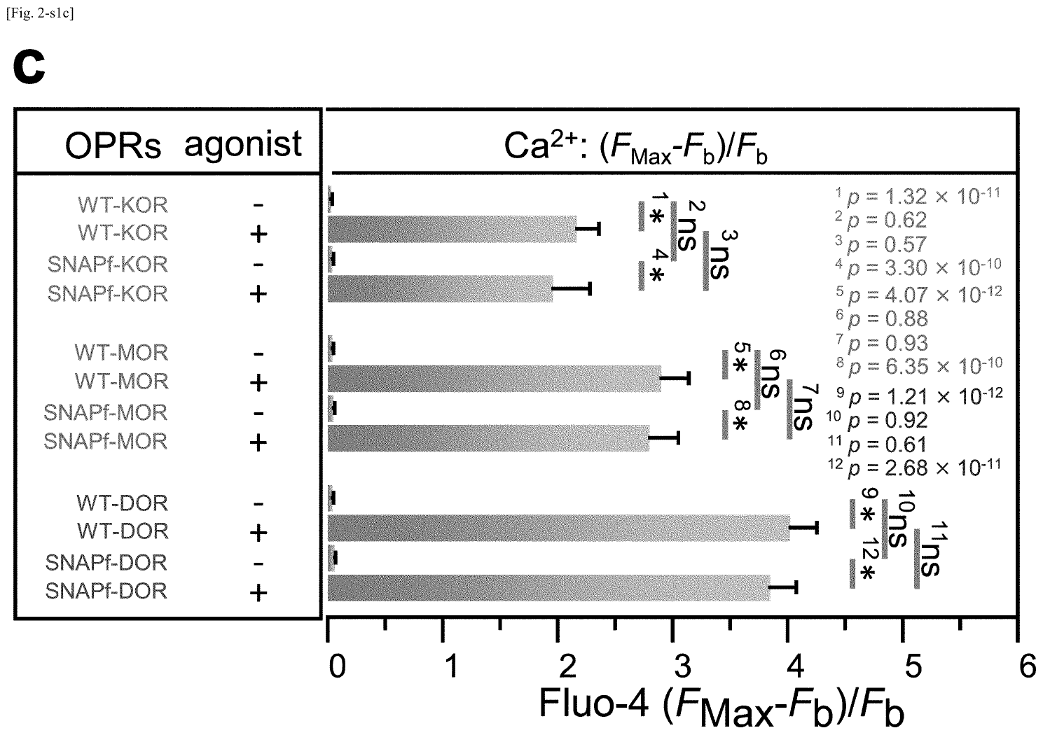

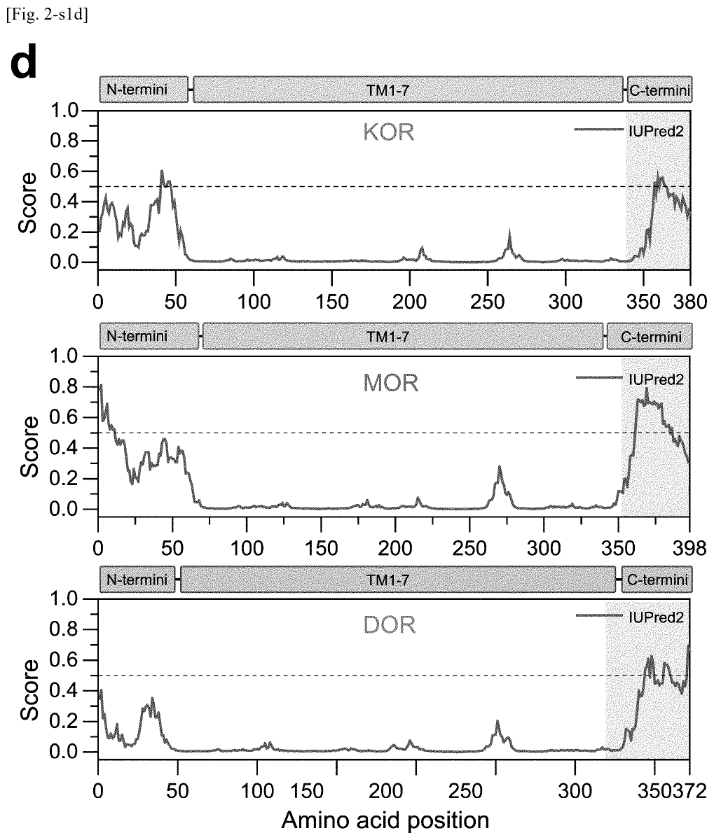

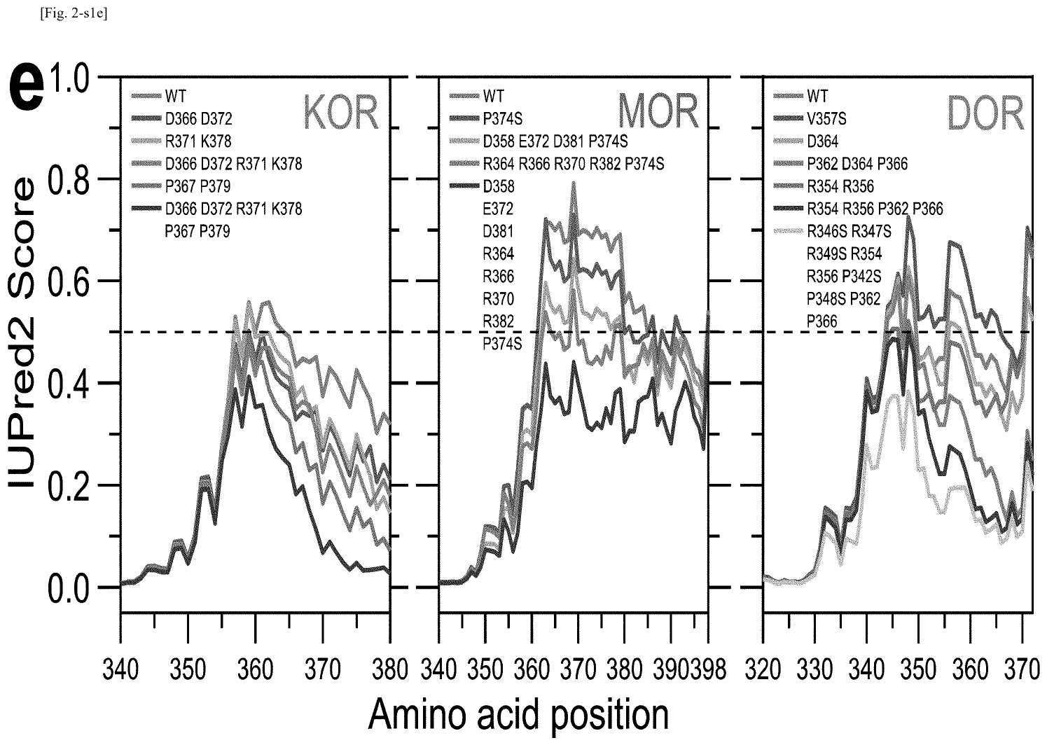

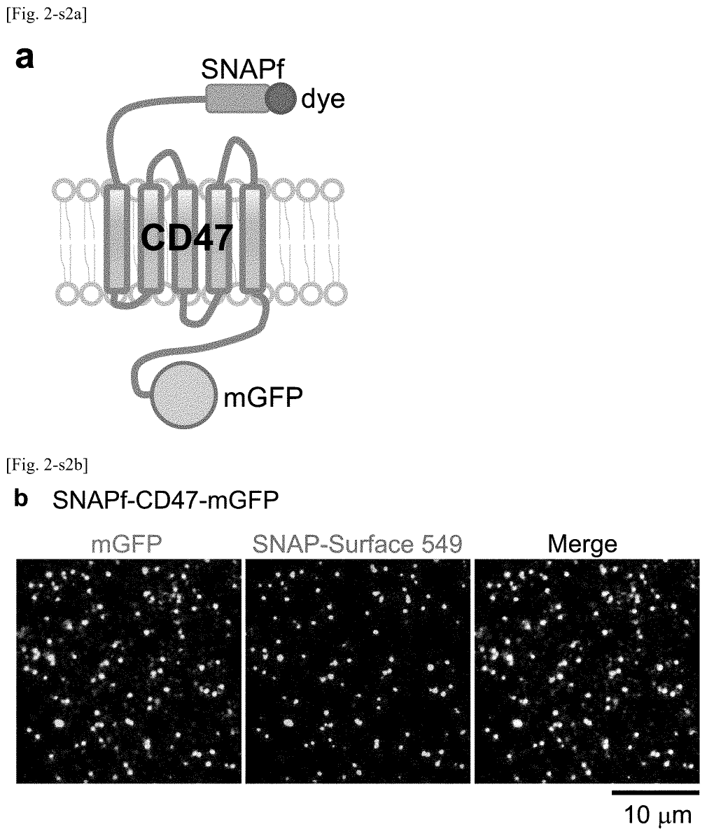

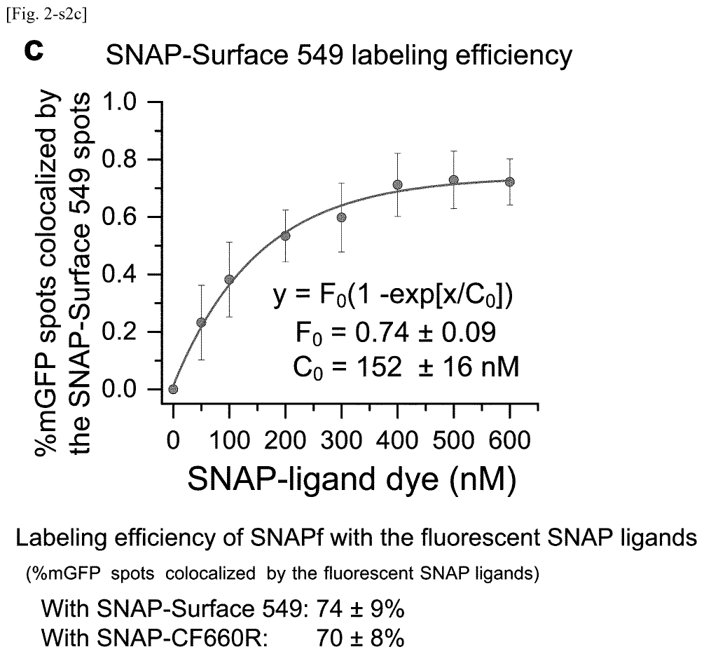

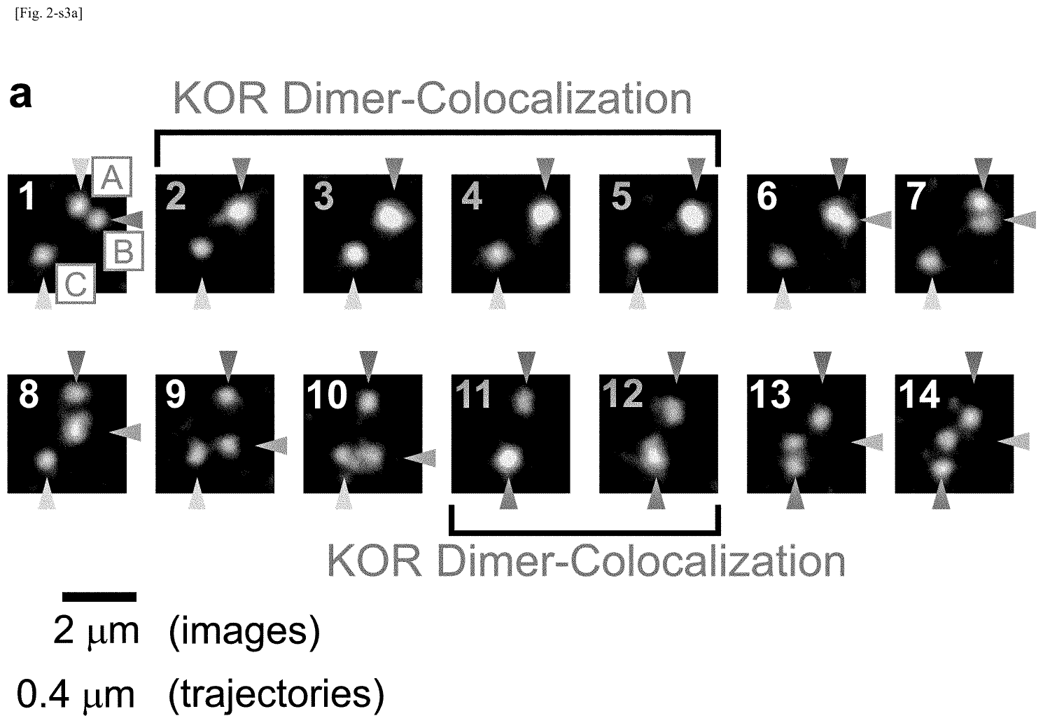

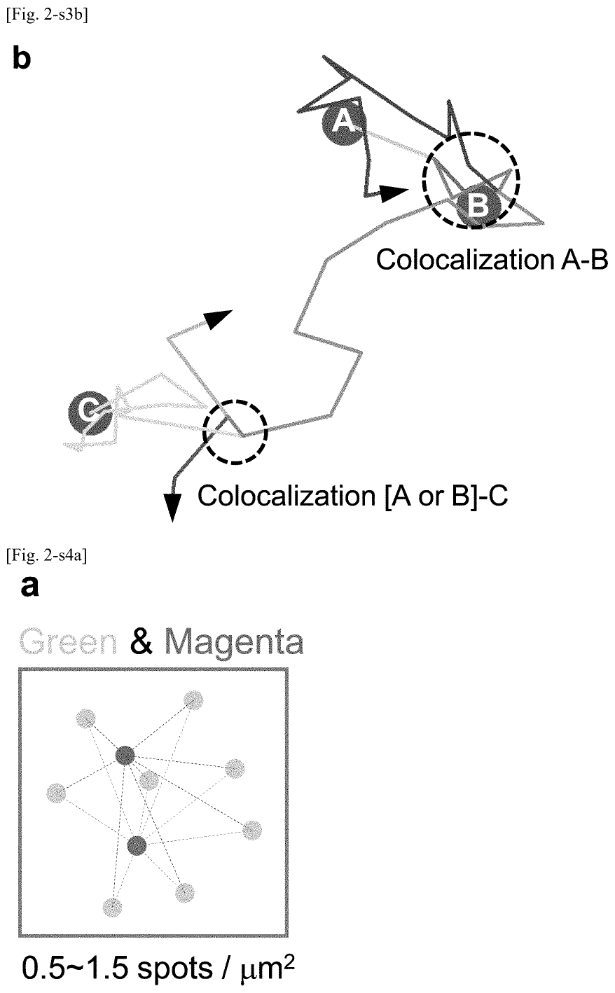

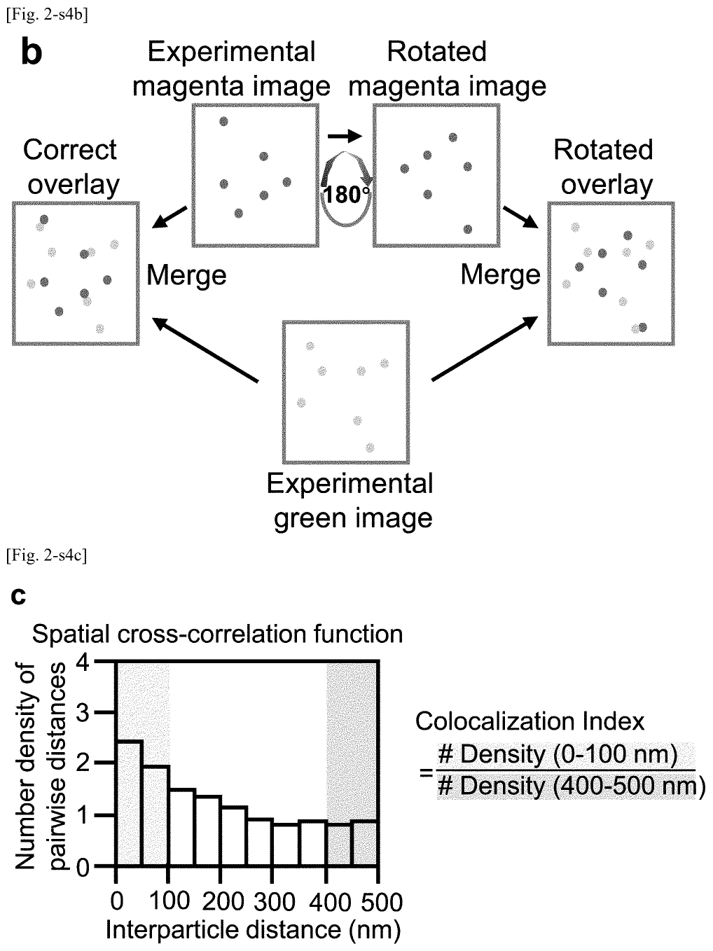

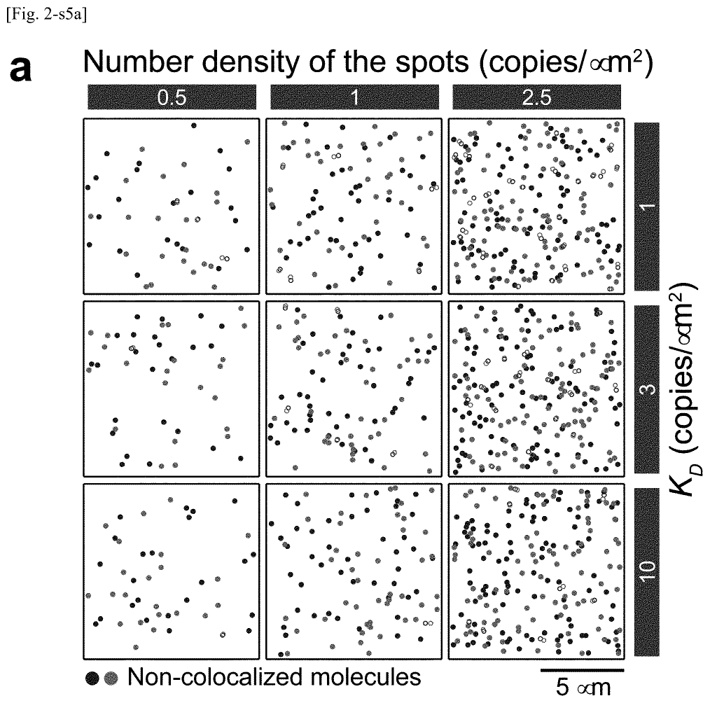

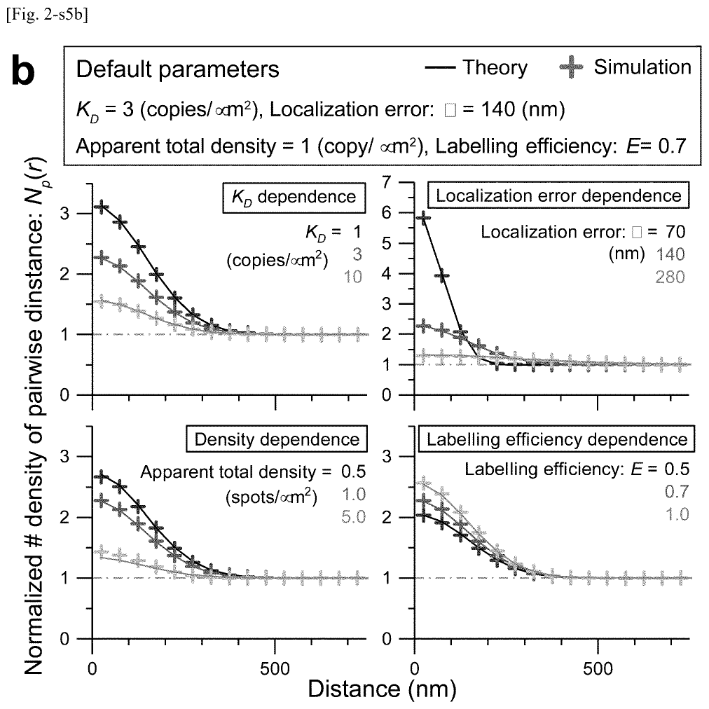

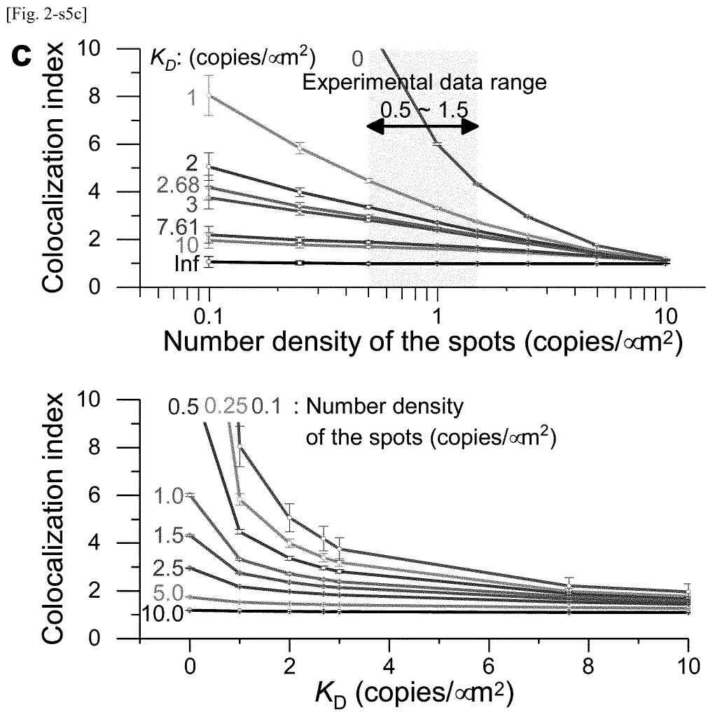

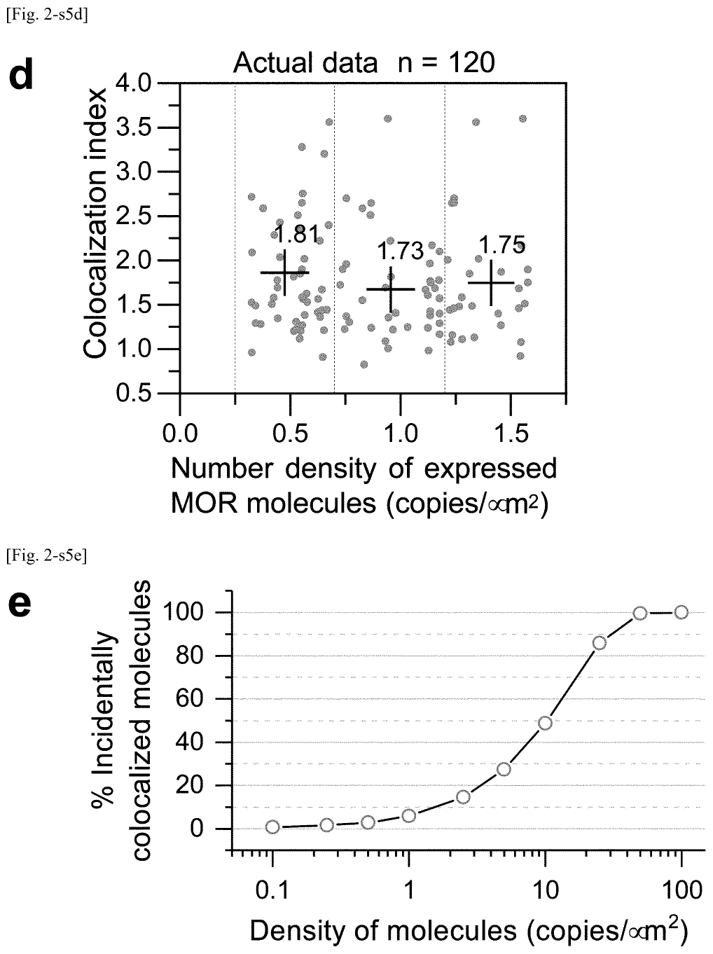

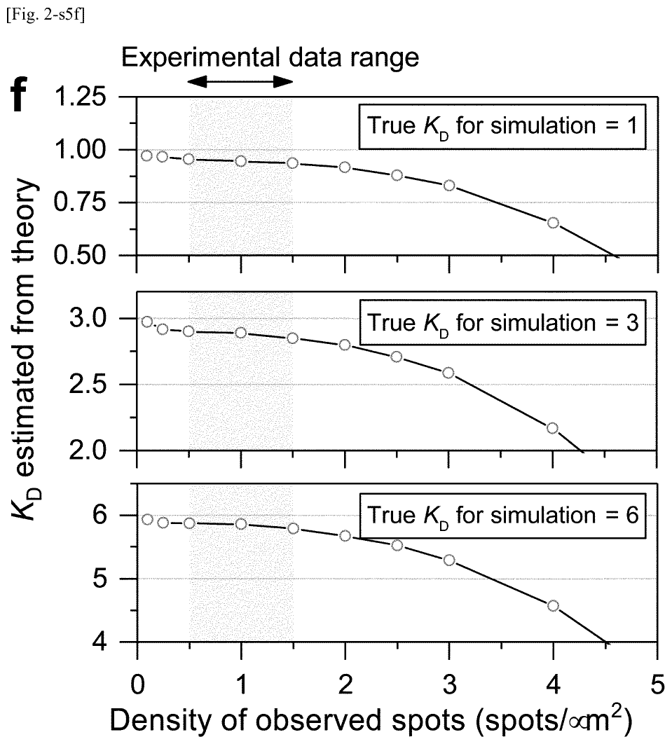

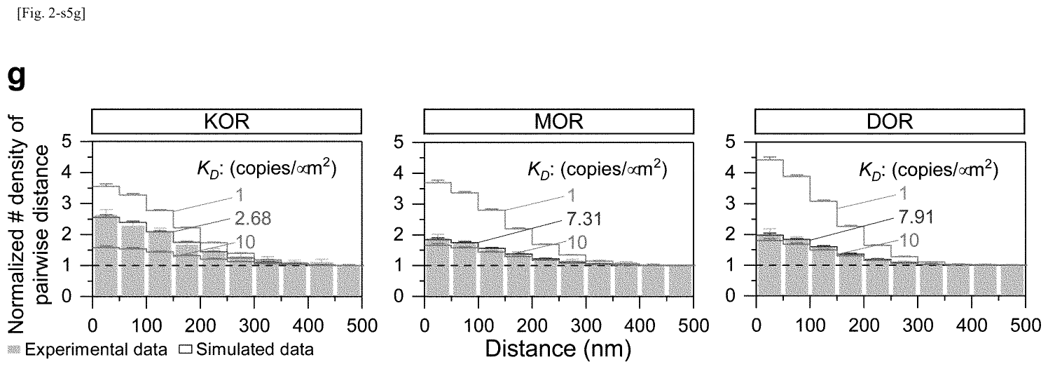

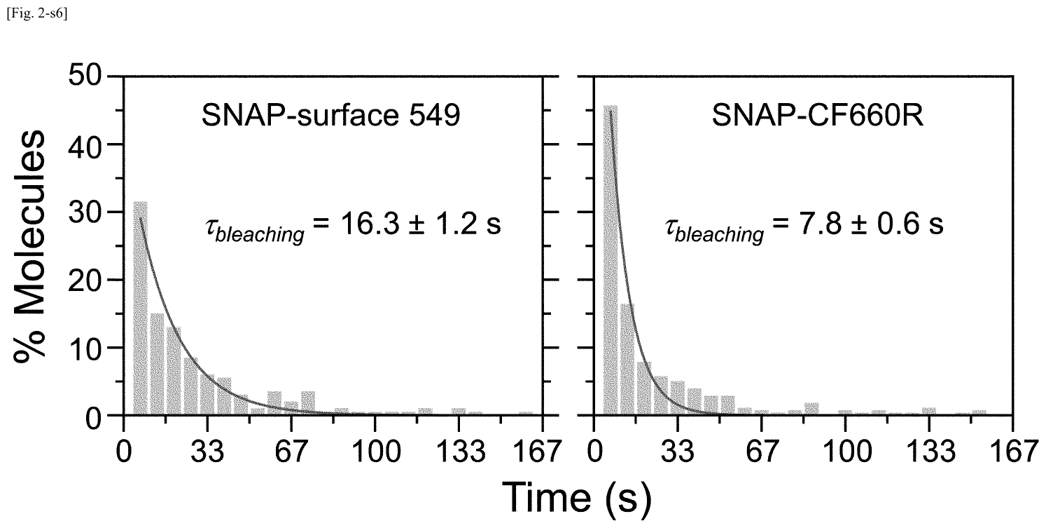

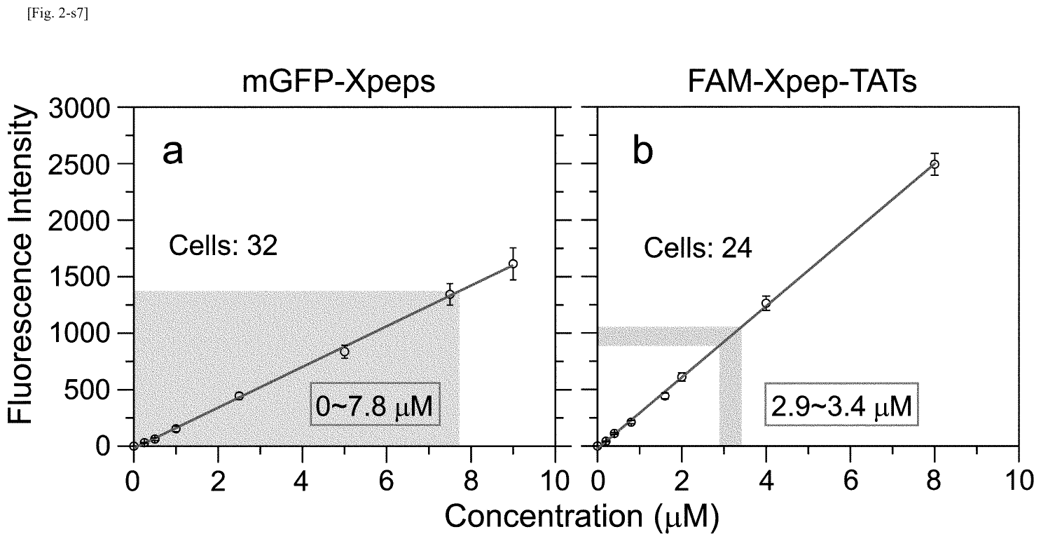

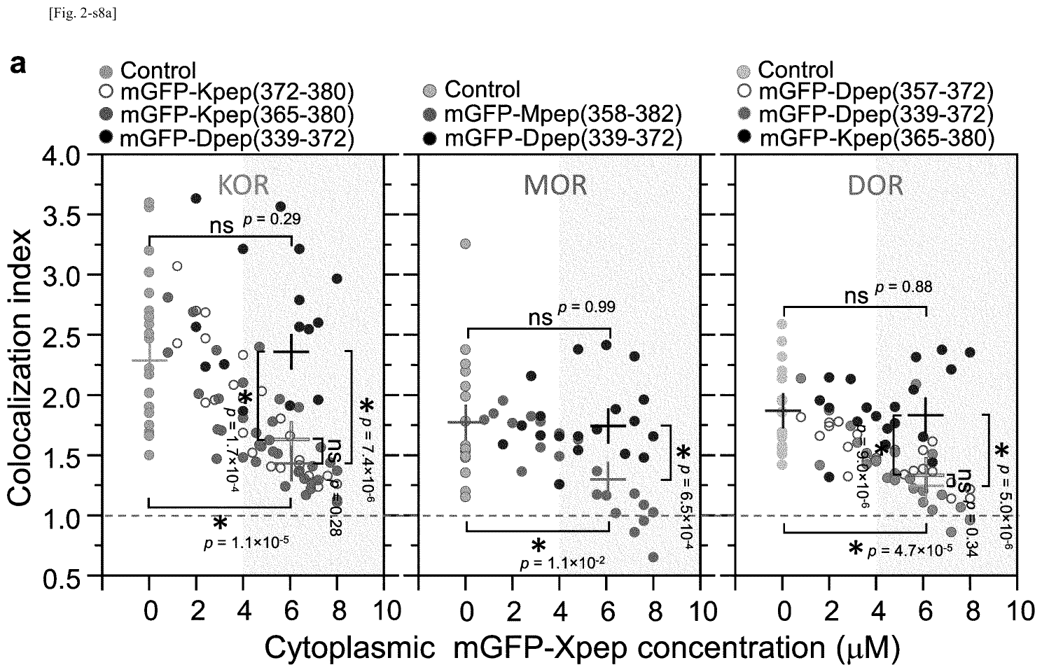

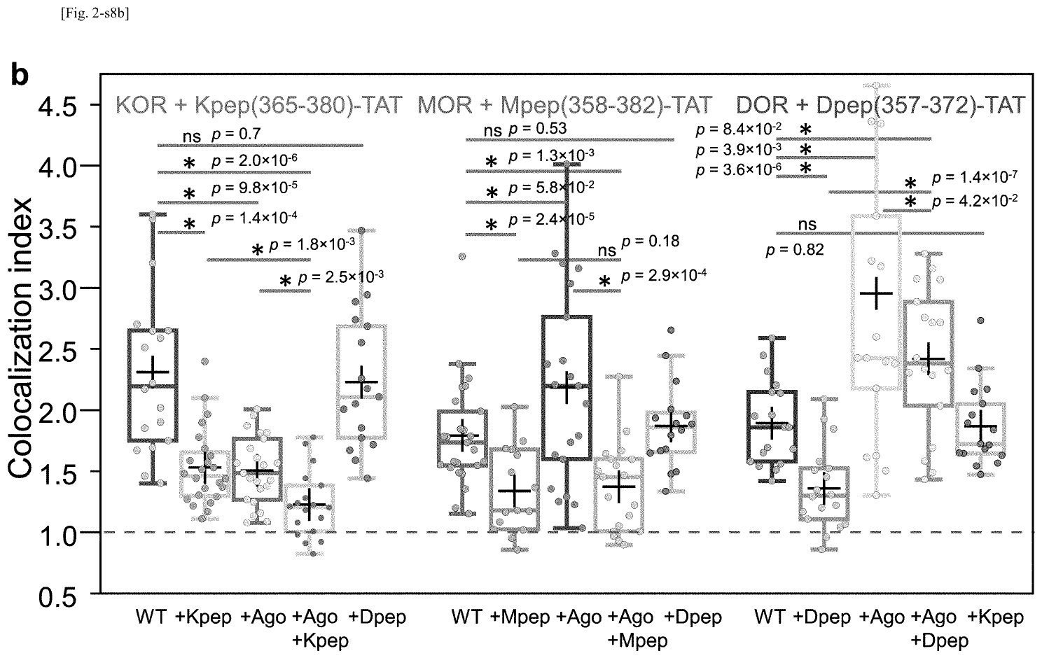

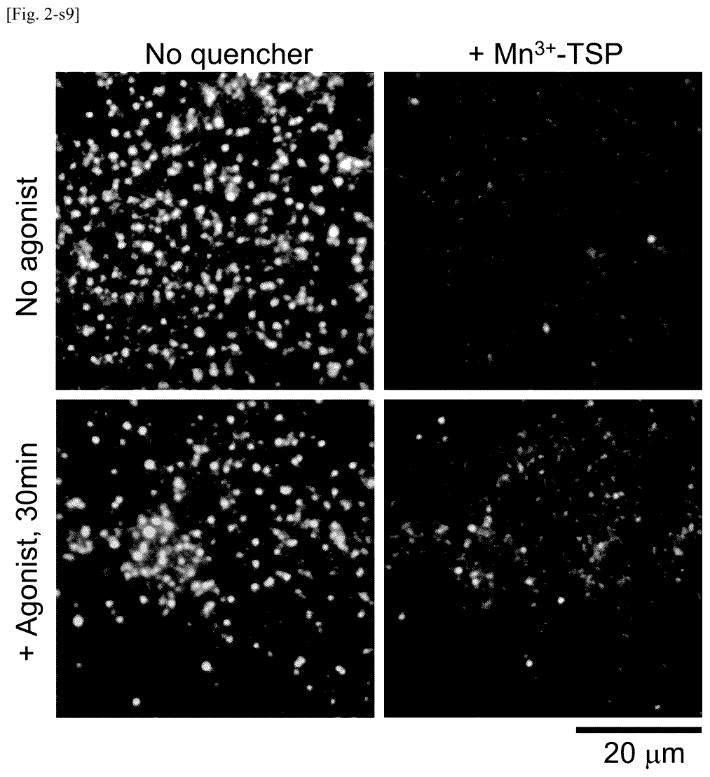

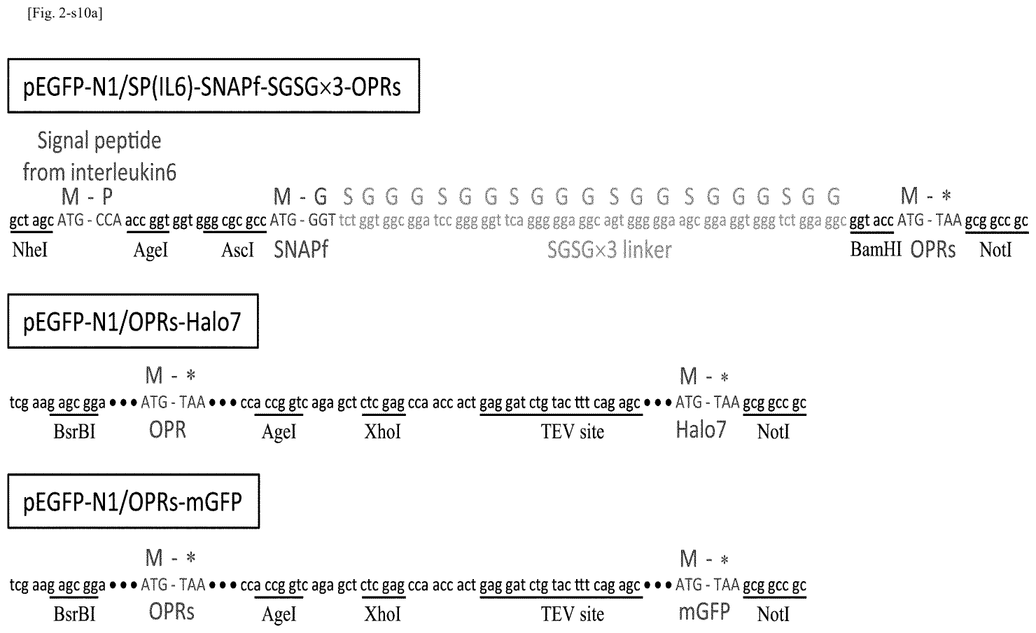

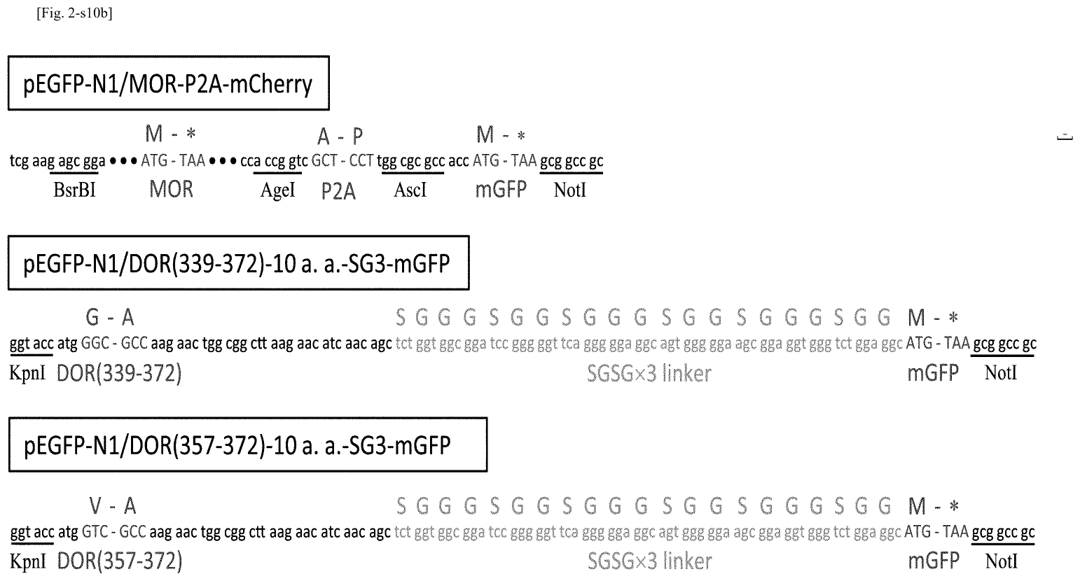

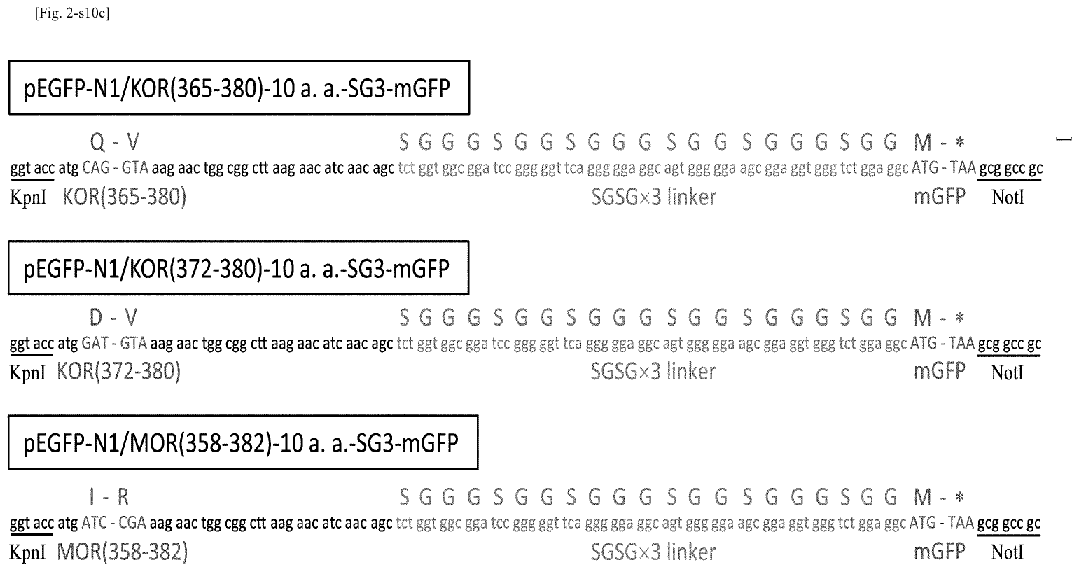

The fluorescently-tagged OPRs expressed in the PM of CHO-K1 cells, which we used throughout this study, were functional (Extended Data FIG. 1; >75% labeling efficiencies) and observed at the level of single molecules in two colours at video rate (30 Hz) at 37° C. (conditions used throughout this research). Virtually all of the OPR fluorescent spots exhibited diffusion in the PM, and when DOR and MOR (and DOR and KOR) were expressed in the same cell, they exhibited frequent temporary colocalisation and co-diffusion, consistent with transient heterodimer formation (FIG. 1-1a). For simple quantification of the tendencies of OPR heterodimer formation, we employed the colocalisation index, a parameter based on the pair cross-correlation function (PCCF of the green and magenta spots), which is a measure of finding molecule B (for example, a green spot) within a 100 nm radius circle around molecule A (for example, a magenta spot) relative to farther distances of 400-500 nm (as a negative control, the video frames in the green channel were rotated 180° before overlaying; Extended Data FIG. 1-2). The colocalisation index indicated DM and DK heterodimerisations, but not KM dimerisation (FIG. 1-1b).

The heterodimer lifetime was determined in the following way. Each time we detected a colocalisation event of spots with different colors, its duration was measured, and after sufficient numbers of colocalisation durations were obtained (>3,000 events in ≥20 cells), we produced the distribution of colocalisation durations (histogram; FIG. 1-1c). We found that the histogram could be fitted by the sum of two exponential decay functions (shorter and longer decay time constants; τ₁ and τ₂, respectively), as predicted from the theory described in the companion paper, which showed that τ₁ represents the lifetime in which non-associated molecules track together by chance and τ₂ provides the heterodimer lifetime (the lifetime of actual binding of two molecules, according to the theory we developed): 260±11 ms for DM heterodimers, and 240±10 ms for DK heterodimers after corrections for photobleaching lifetimes (FIG. 1-1c; Extended Data 3). The histogram for the MK colocalisation durations did not exhibit any τ₂ component, consistent with the lack of detectable heterodimers for this pair (only incidental colocalisation with a lifetime of T); FIG. 1-1c). These results are summarised in FIG. 1-1d.

Since MK heterodimers are not detectable under our observation conditions, these results suggest that the amino-acid (aa) sequences responsible for heterodimerisations might be mainly located in particular OPR domains with lower sequence identities/homologies among the three OPRs. The candidate sites are summarised in FIG. 1-1c. The aa sequence homology in the cytoplasmic C-terminal domains is lower, but since they are involved in the OPR homodimerisations, they are not included in this figure.

### EL3 Domains are Responsible for DK Dimerisation

As shown in FIG. 1-1e, the aa homologies in the OPRs' N- and C-terminal domains are low, suggesting that these domains might be involved in DK heterodimerisation. Therefore, we produced the N- and C-terminal deletion mutants of DOR and KOR and examined their heterodimerisation with the wild-type (WT) KOR and DOR, respectively (FIG. 1-2a). They failed to affect DK heterodimerisation, indicating that the N- and C-terminal domains are not involved in this process.

Since MK heterodimerisation does not occur, we replaced various MOR domains with the corresponding DOR domains (FIG. 1-1c), and examined whether any DOR domain in the MOR can induce dimers with KOR (FIG. 1-2b left). Replacing the fourth transmembrane domain (TM4), as suggested previously (Filizola and Weinstein, Protein Eng 2002; Liu and Wang, R. J. Computer-Aided Mol Design 2009), slightly increased the colocalisation index, whereas replacing the extracellular loop 2 (EL2) failed to increase the index. However, replacing the extracellular loop 3 (EL3) of MOR with that of DOR increased the colocalisation index with KOR to a level close to that for DK heterodimerisation (FIGS. 1-2b and 1-1b), indicating that the DOR's EL3 plays an important role in the interaction with KOR. Furthermore, we replaced various DOR domains with the corresponding MOR domains, and examined which MOR domain(s) in DOR blocked the interaction with KOR (FIG. 1-2b right). The DK heterodimerisation was only greatly reduced when DOR's EL3 was replaced by MOR's EL3, indicating that DOR's EL3 plays a key role in DK dimerisation.

Furthermore, replacing the N-terminus domain of MOR with that of DOR failed to induce dimers (FIG. 1-2b, left, bottom) and replacing the N-terminus domain of DOR with that of MOR failed to block DM heterodimerisation (FIG. 1-2b, right, bottom). These results indicate that the N-termini of DOR and KOR are not involved in DK heterodimerisation, consistent with the N-terminus deletion data.

The lifetime decay histogram for MOR (EL3-DOR)-KOR exhibited a significant fraction of the τ₂ component as compared with that for WT-MOR-WT-KOR, indicating the critical involvement of DOR's EL3 in DK heterodimerisation (FIG. 1-2c). Meanwhile, the τ₂ value for MOR (EL3-DOR)-KOR was not prolonged to that for WT-DOR-WT-KOR, suggesting that, in addition to the EL3, other interaction sites exist for the DK pair (FIG. 1-2c).

### EL3-Based Peptides Block DK Heterodimers

To further explore the involvement of the DOR's EL3 in DK dimerisation, and to determine whether the KOR's EL3 is also involved, we produced soluble peptides containing their EL3 aa sequences (FIG. 1-1e), and added them (1 μM, final concentration) to cells stably co-expressing DOR and KOR. Both peptides significantly reduced DK dimerisation, whereas the peptide containing the MOR EL3 sequence did not (FIG. 1-2d). These results indicate that EL3 is critically involved in DK dimerisation.

However, since the colocalisation indices did not reach 1 (no colocalisation except for incidental colocalisation), these results suggest the possibility that other domains, such as transmembrane domains, as suggested previously (Jacobs et al., Mol. Pharm. 2018), might also be involved in DK dimerisation. To test this possibility, we examined the dimer formation of KOR with DOR's TM1 and TM4 (FIG. 1-2c). These DOR TM domains indeed indicated slight tendencies to form heterodimers with KOR.

### N-Terminal Domains are Responsible for DM Dimerisation

Although the first transmembrane domain (TM1) of MOR is reportedly involved in DM heterodimerization (He et al., Neuron 2011), in brain cortical membranes prepared from wild-type mice, the monoclonal antibody specifically recognising the DM heterodimers bound to the cell surface and blocked the DOR antagonist-mediated increases in the MOR agonist-mediated signaling (Gupta et al., Sci. Signal. 2010), suggesting that the extracellular domains of MOR and DOR could be involved in DM heterodimerisation. Since the aa homologies in the OPRs' N-terminal domains are low (FIG. 1-1c), and the N-terminal domain does not participate in DK heterodimerisation, we examined the involvement of the N-terminal domains of both DOR and MOR in DM heterodimerisation. At first, we systematically deleted partial sequences from the N-terminal domains of DOR and MOR. We found that DOR's aa22-27 and MOR's aa32-61 play key roles in DM heterodimerisation; i.e., these domains bind to each other to form DM dimers (FIG. 1-3a). Consistently, the histogram of the colocalisation durations for DORΔ22-42 with WT-MOR and that for MORΔ32-61 with WT-DOR both exhibited the existence of only small fractions of the τ₂ components, with much shorter dimer lifetimes (88±12 ms and 91±8 ms, respectively; FIG. 1-3b).

However, we noted that the blocking by deletion mutants was incomplete in all cases. The colocalisation indices were decreased to 1.1˜1.2, but not completely to 1.0. Therefore, we again took advantage of the fact that MK heterodimerisation does not occur. Accordingly, we replaced various DOR domains with the corresponding KOR domains (FIG. 1-1e) and examined whether any KOR domain in the DOR can induce dimers with KOR (FIG. 1-3c). Replacing the N-terminus domain of DOR with that of KOR greatly reduced the colocalisation index with MOR (FIGS. 1-3c and 1-1b), consistent with the results that DM heterodimerisation is mediated by the N-terminus domains of both DOR and MOR (FIGS. 1-3a and b). Replacing DOR's TM1 or TM4, but not TM6, with the corresponding KOR TMs mildly decreased the colocalisation index with WT-MOR, and replacing MOR's TM1 domain with KOR's TM1 domain also slightly reduced the colocalisation index with WT-DOR (the major interaction sites of the DK pair would be the EL3 domains; FIG. 1-3c and FIG. 1-2). These results indicate that, despite their quite high homologies, DOR's TM1 and TM4 interact with MOR and MOR's TM1 interacts with DOR, as predicted by computational modelling (Filizola and Weinstein, Protein Eng 2002; Liu and Wang, R. J. Computer-Aided Mol Design 2009) and a physiological assay (Jacobs et al., Mol. Pharm. 2018), respectively, although the efficacies of the TM-TM interactions for dimerisation appeared to be weaker than those of the N-terminus interaction and the EL3 interaction, respectively.

In a previous co-immunoprecipitation assay, the aa53-99 sequence of MOR, which contains part of the N-terminus region (aa53-70), MOR's TM1 (aa71-94), and an additional five amino acids that follow TM1 (95-99) (which the authors called the TM1 region of MOR, but we will call N-term+TM1 to avoid confusion), was found to be responsible for the DM heterodimerization (He et al., Neuron 2011). This result is consistent with our observations that replacing MOR's TM1 domain with KOR's TM1 domain only mildly reduced DM heterodimerisation (because DKs mainly interact at their EL3 domains).

### Peptide-Based DM Heterodimer Blockers

The aa sequences deleted from or replaced in the wild-type (WT) OPRs, employed to obtain the results shown in FIG. 1-2a-c, might not represent the true interaction sites for heterodimerisation. Instead, the inhibition of heterodimerisations by the deletion (N-termini) or replacement (TM) mutations might have been induced by conformational changes of the true interaction sites, caused by these deletions and replacements.

To examine these possibilities, first, we produced the soluble-peptides containing the same aa sequences as those of the deleted parts of the mutants, and added them to cells stably co-expressing WT-DOR and MOR. The DOR (MOR) peptides with the aa sequences possibly involved in DM dimerisation were called Dpep(m-n)D-M (Mpep(m-n)MD), where m and n indicate the amino acid numbers in the original OPR sequences (FIG. 1-3d). The various DpepDMs and MpepMDs lowered the colocalisation indices, with the largest reductions found with Dpep(20-42)DM and Mpep(32-61)MD, consistent with the results obtained with the deletion mutants (FIG. 1-3a). The dependence of the colocalisation index on the Dpep(20-42)DM concentration shown in FIG. 1-3d (right, top) indicates that the affinity (K_{D}) of the D-M heterodimers is <0.1 μM in 3D. However, this could not be readily converted to the number in 2D, where the actual DM interactions would primarily occur. The Dpep(20-42)DM addition reduced the heterodimer lifetime to 124 ms (from 260 ms in its absence) without varying the τ₂ fraction (41%) (FIG. 1-3d, right, bottom), supporting the DM dimerisation via aa20-42 in DOR.

Second, we expressed MOR's N-term+TM1 and MOR's TM1 (DOR's TM1, 4, and 5 as previously suggested (Filizola and Weinstein, Protein Eng 2002; Liu and Wang, R. J. Computer-Aided Mol Design 2009) conjugated with the SNAPf tag, and examined whether they can interact with WT-DOR (WT-MOR) and compete with the DM interaction (FIG. 1-3c-g). Both MOR's N-term+TM1 and MOR's TM1 colocalised with DOR as well as WT-MOR. Meanwhile, DOR's TM1, TM4, and TM5 exhibited colocalisation with MOR, but to a much lower extent than WT-DOR (FIG. 1-3f). MOR's N-term+TM1 and MOR's TM1 both lowered the DM colocalisation, but the N-term+TM1 competes with DM dimers more effectively (FIG. 1-3g). DOR's TM4 competed with DOR for DM binding, but the efficiencies were lower (FIG. 1-3g). These results indicate that for DM heterodimerisation, the N-terminal domains of both DOR and MOR are important, but MOR's TM1 domain, which might interact with DOR's TM1, 4, and 5 domains, also plays a role.

### Agonists Variously Affect DM Heterodimerisation

We next examined how the inhibition of DM heterodimerisation by the peptide blockers affects the agonist-induced intracellular signal and internalisation of DOR and MOR (FIG. 1-4a). As representative agonists, we employed the MOR-agonists DAMGO and morphine (0.5 μM(Yekkirala et al., ACS Chemneuro. 2009)), and the DOR agonist SNC-80 (0.5 μM(Metcalf et al., ACS Chemneuro. 2012)), which would specifically and rapidly bind to MOR and DOR on the cell surface. We focused on the initial agonist effect without excessive complications by the later signaling events, and thus observed DM heterodimerisation within 5 min after the agonist addition. The DAMGO (morphine)-bound MOR exhibited more (less) heterodimerisation with DOR; i.e., a higher (lower) colocalisation index (FIG. 1-4b), with a prolonged (shortened) heterodimer lifetime (FIG. 1-4c). In the presence of Dpep(20-42)DM, the colocalisation indices and the lifetimes of heterodimers for DOR and agonist-bound MOR (for both DAMGO and morphine) were significantly reduced (FIG. 1-4b, c). Meanwhile, after the addition of 0.5 μM SNC-80, DOR internalisation occurred very quickly (FIG. 1-4d), which prevented us from determining the colocalisation indices and heterodimer lifetimes of MOR and SNC-80-bound DOR.

### Peptide Blockers Suppress MD Co-Internalisation

In cells stably expressing only MOR, only DOR, and both MOR and DOR (called M cells, D cells, and MD cells, respectively), OPR internalisation at a low physiological expression level of ˜1 copy/μm² was monitored by using the membrane-impermeable fluorescence quencher and evaluating the percentages of the OPR molecules remaining in the PM (see the Methods).

The time courses of the OPR numbers remaining in the PM were examined in both the presence and absence of 0.5 μM agonists and 10 μM DpepDM in the medium (FIG. 1-4d). The time course of the MOR/DOR internalisation could be operationally fitted by a single exponential function, providing the fractions of internalised MOR/DOR detectable by 60-min observations and their residency lifetimes in the PM.

MOR internalisation was hardly detectable without the agonist addition in M cells and MD cells. The addition of the MOR agonist DAMGO induced the MOR internalisation more effectively in MD cells (51.1%) than in M cells (31.8%). DOR internalisation was induced by the MOR agonist DAMGO in MD cells (13.3% vs 4.3% without DAMGO addition) (FIG. 1-4d, top row; Supplementary Tables 1 and 2), suggesting the co-internalisation of DOR and MOR. However, morphine (another MOR agonist) hardly affected any of these internalisation processes (FIG. 1-4d, middle row). These results are consistent with previous observations (Derouiche et al., Molecules 2020). The addition of Dpep(20-42)DM to MD cells significantly reduced the DAMGO-induced internalisations of both MOR and DOR (both the fractions and rates), probably due to the suppression of DM co-internalisation (He et al., Neuron 2011).

The addition of the DOR agonist SNC-80 induced dramatic DOR internalisation in D cells and MD cells, as well as MOR internalisation in MD cells, again suggesting the occurrence of MD co-internalisation (FIG. 1-4d, bottom row; Supplementary Tables 1 and 2). The addition of Dpep(20-42)DM to MD cells significantly reduced the SNC-80-induced internalisation of both MOR and DOR (both the fractions and rates), probably due to the suppression of DM co-internalisation.

### DM Heterodimers Enhance MOR Signals

The agonist-induced cytoplasmic signal was evaluated by monitoring Ca²⁺ mobilisation (FIG. 1-4c, f; see the Methods). The additions of DAMGO and morphine induced intracellular Ca²⁺ mobilisation in M-cells, but not in D- and K-cells, and the Ca²⁺ mobilisation was greatly enhanced in MD-cells. The presence of Dpep(20-42)DM in the medium of the MD cells moderated the enhancement of the Ca²⁺ signal in the presence of DOR in MD cells. These results indicate that DM heterodimerisation enhanced the MOR-agonist-induced MOR signals.

### Soluble Heterodimer Blocker Suppresses Morphine Tolerance

The soluble DM heterodimer blocker peptide Dpep(20-42)DM reduced the development of antinociceptive tolerance to morphine in mice, as examined by the tail-flick test. The peptide dissolved in artificial cerebrospinal fluid (aCSF) was applied continuously to lateral ventricle (10 μg/day) for 2 days prior to the daily subcutaneous administrations of morphine (10 mg/kg/injection; one injection/day) (FIG. 1-4g). The analgesic effect of morphine was facilitated by Dpep(20-42)DM between 60 and 120 min after the morphine application on all days (days 1, 5, 8, and 11; Extended Data FIG. 6). Due to the decreased analgesic effect of morphine for the aCSF group during the experiment, on day 11, the difference from the peptide group became ˜3-fold at 120 min after the morphine application (FIG. 1-4h).

### Soluble Heterodimer Blocker Suppresses Morphine Tolerance.

Furthermore, the Dpep(20-42)DM application reduced the tolerance to morphine. At 8 days after the morphine treatment, the analgesic effect of morphine was reduced in the control aCSF-treated mice. In contrast, the antinociceptive effect of morphine in peptide-treated mice was maintained at ˜70% of the initial effectiveness for 8-11 days (FIG. 1-4i). These results suggest that the disruption of the DM dimers by the peptide prevented the development of antinociceptive tolerance to morphine in the brain.

TABLE 4
Table C. P values for the results shown in FIG. 1-1B using
Tukey's multiple comparison test (n ≥ 20 cells).

| | DOR-MOR | MOR-KOR | KOR-DOR |
|---|---|---|---|
| DOR-MOR | NA | 3.72 × 10⁻⁹ | 6.01 × 10⁻¹ |
| MOR-KOR | 3.72 × 10⁻⁹ | NA | 3.53 × 10⁻⁹ |
| KOR-DOR | 6.01 × 10⁻¹ | 3.53 × 10⁻⁹ | NA |
| Rotated data | 8.86 × 10⁻⁹ | 8.23 × 10⁻¹ | 1.21 × 10⁻¹¹ |

DpepDM and DpepDM-TAT toxicity was assessed in T24 cells which are human bladder carcinoma cell lines after a 48-h exposure at concentrations (50 μM, 100 μM, and 200 μM) administered to the cells, using the LDH assay kit from Abcam according to the manufacturer's specification. Cytotoxicity was measured by subtracting LDH content in remaining viable cells from total LDH in untreated controls. DpepDM and DpepDM-TAT showed no toxicity to T24 cells at all even at the very high concentration of 200 μM (FIG. 1-4j).

DpepDM and DpepDM-TAT toxicity was assessed in the primary culture of hippocampal neurons (DIV8, DIV12, and DIV14) after 48, 96, and 144-hour exposures at a concentration of 100 μM administered to the cells, using the LDH assay kit from Abcam according to the manufacturer's specification. Treatments of neurons with Trition-100 (1%) and DMSO (10%) for 48 h were used as positive controls. DpepDM and DpepDM-TAT showed little toxicity to neurons even at the very high concentration of 100 μM (FIG. 1-4k).

It is surprising that the peptides of the present invention did not show any toxicity at very high concentrations of 100 μM and 200 μM, whereas common synthetic peptides usually show strong toxicity at concentrations of 10-50 μM (Front. Microbiol. 11:1146.doi: 10.3389/fmicb.2020.01146; Scientific Reports, (2018) 8:1763, DOI:10.1038/s41598-018-19434-7).

### DISCUSSION

Here, using advanced single-molecule imaging and analysis, we confirmed the formation of DM and DK heterodimers and the lack of KM dimers at physiological expression levels of ˜1 copy/μm². Quite unexpectedly, the DM and DK heterodimers are both transient metastable dimers with a lifetime of ˜250 ms, forming and dispersing continually, as also found for homodimers of other GPCRs and OPRs. As key interaction sites, we identified DOR's aa20-42 and MOR's aa32-61 in the N-terminal domains for the DM dimers, and the short EL3s of DOR and KOR for the DK dimers, in addition to the previously found/proposed various TM domain interactions. These results might provide the sites for both stronger specific interactions and weaker basic interactions (He et al. Neuron, 2011), as suggested in the “rolling interface” model (Dijkman et al. Nat. Commun. 2018; Johnston et al. Biochem. 2011), to support the heterodimerisation.

The peptides with the aa sequences identified as the binding sites confirmed these dimerisation sites located in the extracellular domains of OPRs. These results are consistent with the development of the heterodimer-selective antibody that suppresses DM heterodimerisation by binding to the extracellular domains (Gomes and Devi, PNAS 2013). The Dpep(20-42)DM peptide blocker reduced the DM co-internalisation. The agonist-induced MOR signal is greatly enhanced upon DOR co-expression, as expected from previous reports (Gomes et al., JN 2000; Yekkirala et al., ACS Chem. Neurosci. 2010). We found that this DOR-induced signal enhancement was suppressed by the Dpep(20-42)DM addition.

Dpep(20-42)DM suppressed morphine tolerance development in mice. A previous study, in which MOR's N-term+TM1 linked to the TAT peptide was systemically applied by intraperitoneal infusion quite acutely (three injections within 2.5 h, 10 mg/kg/injection), revealed that the injected membrane-inserting peptide disrupted the DM interaction in the mouse spinal cord and reduced the antinociceptive tolerance to morphine (He et al. Neuron 2011). The DM dimer blocking peptide Dpep(20-42)DM developed here is much smaller and soluble, and thus could be used complementarily with the previous N-term+TM1 linked to the TAT peptide.

Previously, the development of antinociceptive tolerance to morphine was considered to be solely due to the down-regulation of MOR by internalisation, which is enhanced by the DM interaction. However, morphine-induced MOR internalisation might be quite limited, as shown here (FIG. 1-4d) and in a previous study (Moller, J. et al. Nat. Chem. Biol. 2020). Meanwhile, the morphine-induced cytoplasmic signal is enhanced by the co-expression of MOR and DOR in the cell (FIG. 1-4f). Therefore, the development of antinociceptive tolerance to morphine might also involve MOR's downstream signals. These results support an OPR drug administration strategy for reducing the development of tolerance, addiction, and dependence on morphine and other analgesics, by reducing OPR heterodimer formation by using heterodimerisation-blocking drugs, including blocking peptides (Gupta et al., Sci. Signal. 2010; He et al. Neuron 2011).

### <Example 2> Homodimer Blocker

### Methods

cDNA Construction

All of the newly generated cDNA constructs and other constructs obtained from outside sources, including gifts and constructs from commercial sources, were examined and confirmed by DNA sequencing. The cDNAs encoding rat KOR and DOR were gifts from Prof. H. Takeshima of Kyoto University. The cDNA encoding rat MOR tagged with GFP was a gift from Dr. R. Schulz of the University of Munich, Germany⁵⁴. The mCherry was a gift from Prof. R. Y. Tsien of the University of California San Diego⁵⁵. The cDNAs encoding SNAPf and mGFP (A206K) were obtained from Promega and Clontech, respectively. When the tag protein SNAPf was attached to the N-terminus of the OPRs, an additional signal sequence of interleukin 6 was attached to the N-terminus of the tag protein and a 21 amino acid linker (SGGGSGG×3) was inserted between the OPRs and the tag protein. A Site-Directed Mutagenesis Kit (New England Biolabs) was used to generate the cDNAs encoding the OPR point mutants. For the details of the cDNA constructs including the linker sequences, see FIG. 2-s10.

### Cell Culture, Transfection, and Microscope Observations

CHO-K1 cells (Dainippon Pharma) and T24 cells (gift from Prof. M. Sokabe of Nagoya University)⁵⁶, confirmed free of mycoplasma contamination by MycoAlert (Lonza), were routinely cultured in Ham's Nutrient Mixture F12 (Sigma-Aldrich) supplemented with 10% (v/v) fetal bovine serum (FBS, Life Technologies), 100 units/ml penicillin, and 0.1 mg/ml streptomycin, at 37° C. under a 5% CO₂ atmosphere in the incubator. Transfection of CHO-K1 cells with the cDNAs of interest was performed by electroporation, according to the manufacturer's instructions (4D-Nucleofector, Lonza; SF Cell Line solution and program CHO-K1 for CHO-K1 Cells). The transfected cells were seeded in glass-base dishes (35-mm in diameter with a 12-mm diameter glass window, 0.15-mm-thick glass; Iwaki, Tokyo; 2×10⁵ cells/dish) and cultured for 24-48 h before fluorescence microscopy observations. All microscope observations were performed at 37° C. by placing the entire microscope, except for the far ends of the excitation arms and the detection arms, in a home-built microscope environment chamber made with thermo- and electric-field-insulating plastic sheets and equipped with four heating circulators (SKH0-112-OT, Kokensya, Tokyo, Japan). The Ham's F12 medium used for microscope observations was free of sodium bicarbonate and phenol red, and buffered with 2 mM N-[tris(hydroxymethyl)methyl]-2-aminoethanesulfonic acid (TES, Sigma-Aldrich) at pH 7.4 (called Ham's F12 observation medium).

Fluorescence Labeling of OPRs Expressed on the Cell Surface (FIG. 2-s2)

The SNAPf-tagged wild-type and mutant OPRs expressed in the PM (SNAPf tag located at the extracellular N-terminus) were covalently conjugated with SNAP-Surface 549 (New England Biolabs) and SNAP-CF660R (Shinsci Kagaku), by incubating the cells with these fluorescent SNAP ligands at 300 nM simultaneously in the growth medium, at 37° C. in the CO₂ incubator for 30 min. The cells were washed three times with fresh medium (5-min incubation each time), and then the Ham's F12 observation medium was added. The labeling efficiency was determined by using the SNAPf-CD47-mGFP protein expressed in the PM. CD47 is a monomeric 5-pass transmembrane protein expressed in the PM37. We could not use OPRs for this purpose because they form transient dimers, and thus employed a monomeric CD47. Under the conditions employed here, we achieved ≥70% labeling: 74±9% for SNAP-Surface 549 and 70±8% for SNAP-CF660R.

### Single Fluorescent-Molecule Imaging and Tracking in Live Cells

Fluorescently labeled OPRs expressed in the bottom PM (the PM facing the coverslip) at fluorescent-spot number densities of 0.5˜1.5 per μm² (total number of the spots in two colors) were observed at the level of single molecules at 37° C., using a home-built objective lens-type TIRF microscope constructed on an inverted microscope (Olympus IX-83), with a 100×, 1.49 numerical aperture (NA) objective lens, optimized for the present research based on the instrument used previously^{57,58}. OPRs tagged with fluorescent probes were excited with TIR illumination using the following power densities: SNAP-Surface 549 at 561 nm (Coherent OBIS 561-100 LS) at 0.35 μW/μm²; and SNAP-CF660R at 642 nm (Omicron LuxXPlus 640-140) at 0.52 μW/μm². The dual-color images were separated by a dichroic mirror and projected into two detection arms with band-pass filters of 500.0-550.0 nm (ET525/50 m; Chroma) for mGFP, 572.5-647.5 nm (ET600/50 m; Chroma) for the SNAP-Surface 549 dye, and 662.5-737.5 nm (ET700/75 m; Chroma) for the SNAP-CF660R dye. Under these conditions, the localization precision for single dye molecules in living cells (standard deviation assuming the Gaussian distribution) was 60 nm as estimated from the PCCFs. The fluorescence signal in each channel was first detected and amplified by a two-stage microchannel-plate image intensifier (C9016-02MLP24; Hamamatsu Photonics), and the intensified image was projected onto the scientific CMOS camera (C11440-22CU; Hamamatsu Photonics), operated at 30 Hz, which was synchronized with the same intensifier-camera set placed on the other detection arm. The image sequences in each channel were superimposed after correction for spatial distortions, as described previously⁵⁷. The positions (x and y coordinates) of all of the observed single fluorescent-molecules were determined by an in-house computer program, as described previously⁵⁹.

### Evaluating Colocalization Durations

The colocalization of two fluorescent molecules was defined as the event where two fluorescent spots, representing these molecules, become localized within 200 nm of each other, as described previously^{23,57}. Briefly, in single-color experiments using SNAP-Surface 549 (FIG. 2-s3), a cross-correlation analysis was employed²³. Using this method, we found that when two fluorescent spots, each representing a single molecule of SNAP-Surface 549, are located close together, the threshold distance for discriminating one or two spots occurred at 200 nm in the present experimental set up. Using this definition, colocalized trajectories were obtained and colocalization durations were estimated. In simultaneous two-color single fluorescent-molecule tracking experiments, using the dye pairs, the distance between the two molecules was directly measured from the locations (x, y-positions) of each molecule (with different colors). Even when examining pairs of different-colored molecules that are known to be truly associated, the probability of scoring the two molecules as associated is limited by the localization accuracies of each molecule and the accuracies of superimposing the two images. Based on the method developed previously⁵⁷ and the accuracies determined here, we found that, for truly associated molecules, the probability of scoring the two molecules as associated increases to 99% when using the criterion that the molecules lie within 200 nm of each other. Therefore, we used this criterion as the definition of colocalization in simultaneous two-color single-molecule observations. This distance of 200 nm coincided with the definition of colocalization in single-color experiments. Due to this coincidence, in the present research, we defined the colocalization of two fluorescent molecules as the event where the two fluorescent spots representing these molecules become localized within 200 nm from each other.

Each time we found a green-magenta pair located within 200 nm (colocalization), we measured the duration in which their distances remained within 200 nm (colocalized duration). After obtaining the colocalization durations for all of the colocalization events, we generated a histogram (distribution) of colocalization durations. First, the distribution of incidental colocalization durations was obtained by superimposing the magenta image sequences with the 180-degree rotated (doubly flipped) green image sequences (FIG. 2-s4b). We found that the distribution could be fitted by a single exponential function, with a decay time constant representing the incidental colocalization lifetime τ_{inci}. Second, the distribution of colocalization durations was obtained for correctly superimposed magenta and green image sequences. This distribution could be fitted by the sum of two exponential decay functions (τ₁ and τ₂; τ₁<τ₂,). These results are consistent with the theory developed here that predicts the distribution of colocalization durations based on the diffusion equation. Since τ₁ was almost the same as τ_{inci} (FIG. 2-1d), τ₂ provided the colocalization lifetime or homodimer lifetime (after correction for the photobleaching lifetimes of the two fluorescent probes; FIG. 2-s6). The photobleaching lifetimes of the fluorescent probes for 30-Hz observations (τ_{bleach}'s) were 16.3±1.2 s (n=500) for SNAP-Surface 549 and 7.8±0.6 s (n=400) for SNAP-CF660R, adsorbed on the cover glass of the glass-base dish (FIG. 2-s6), and the correction was made by using the equation τ(corrected)=[τ(observed)⁻¹−τ_{bleach}(dye 1)⁻¹−τ_{bleach}(dye 2)⁻¹]⁻¹.

### Evaluating the Colocalization Index: A Quantitative Measure for Colocalization

For the quantitative evaluation of the extent of colocalization (representing both the frequency and lifetime of colocalization events) in simultaneous two-color single-molecule imaging movies, we defined a parameter called the colocalization index (for the results obtained by using two dye molecular species with different excitation/emission wavelengths, we call them green and magenta probes/movies for convenience in this report). This analysis method is essentially based on a pair cross-correlation analysis³⁹, and the detailed method used in this research is explained in FIG. 2-s4.

Monte-Carlo Simulations for Examining the Dependence of the Colocalization Index and the Pair Cross-Correlation Functions (PCCFs) on the Number Density of the Fluorescent Spots (Expression Levels in the PM; FIG. 2-s5)

The colocalization index depends on the number density of fluorescent spots in the PM. In the present investigation, we selected the cells exhibiting fluorescent spot number densities in the PM between 0.5 and 1.5 spots/μm². Therefore, we used Monte-Carlo simulations to examine the extent to which the colocalization index depends on the fluorescent spot number density (FIG. 2-s5). At the same time, we developed the theory to evaluate the dimer-monomer dissociation equilibrium constant K_{D} from the PCCF and the total number of spots in the image.

Treating the Cells with Agonists and FAM-Xpep-TATs

U-50488, DAMGO, and SNC-80 (Sigma-Aldrich), agonists for KOR, MOR and DOR, respectively, were dissolved in DMSO (2 mM), and then diluted with Hanks' balanced salt solution (HBSS, Nissui) buffered with 2 mM TES at pH 7.4 (T-HBSS), at a final concentration of 200 μM. The agonist solution (1 μl) was added to the cells in 1 ml Ham's F12 observation medium (a final concentration of 200 nM).

For the cellular incorporation of FAM-Xpep-TATs (custom-synthesized by CosmoBio), the cells were first incubated with 150 μM pyrenebutyrate (Sigma-Aldrich) in T-HBSS at 37° C. for 5 min, and then 2 mM FAM-Xpep-TAT in T-HBSS was added at a final peptide concentration of 20 μM. After an incubation at 37° C. for 10 min, the cells were washed three times with T-HBSS, and then fresh Ham's F12 observation medium was added to the cells. The presence of FAM-Xpep-TAT in the cytoplasm was confirmed by the addition of the membrane-impermeable quencher, trypan blue⁶⁰.

Confocal Imaging of Live Cells Expressing mGFP-Xpeps and Those with Incorporated FAM-Xpep-TATs

CHO-K1 cells stably expressing OPRs were transfected with mGFP-Xpeps. To identify both the cells expressing mGFP-Xpeps and those that do not, the cells growing on glass-base dishes were incubated with NucSpot Live 650 Nuclear Stain (Biotium), according to the protocol recommended by the manufacturer. After three washes with the complete medium, it was replaced with the Ham's F12 observation medium. The incorporation of the FAM-Xpep-TATs in the cells was performed as described in the previous subsection. Confocal fluorescence images were acquired with the same microscope station used for single-molecule imaging at 37° C., equipped with an Olympus SR10 spinning-disc confocal super-resolution unit (with a Plan-Apochromat 100× oil immersion objective; NA=1.49).

GFP and FAM were excited at 488 nm and detected through a 505-530 nm band pass filter. The NucSpot Live 650 was excited at 642 nm and detected through a 662.5-737.5 nm long pass filter. The concentrations of cytoplasmic mGFP-Xpep and FAM-Xpep-TAT were evaluated using calibration curves, obtained by observing the purified EGFP protein (BioVision) and FAM-Xpep-TATs dissolved in Ham's F12 observation medium at various concentrations, with a focus at 5 μm above the cover-glass surface (FIG. 2-s7).

Observing the Agonist-Induced Ca²⁺ Mobilization in Live Cells (Supplementary FIG. 2-s1d and FIG. 2-6)

Since the OPR expression level varies from cell to cell, the signaling process must be observed at the level of individual cells. The OPRs are coupled to the inhibitory trimeric G protein Gi, which induces the decrease of the cytoplasmic cAMP concentration by inhibiting adenylyl cyclase. However, using the cells with low OPR expression levels employed for single-molecule observations (0.5-1.5 fluorescent spots/μm²), the decrease of the cytoplasmic cAMP levels in individual cells were impossibly difficult to measure. Meanwhile, the increase of the cytoplasmic Ca²⁺ concentrations by the PLCβ activated by the Gq signaling pathway could be measured in individual cells. Therefore, in this assay, Gαq was modified so that OPR could be coupled to Gαq. Namely, the C-terminal five amino-acid sequence of Gαq, ECGLY, was replaced with that of Gαi2, DCGLF (this chimeric protein is called Gqi5), because the Ga protein binds to the specific GPCR by way of its short C-terminal sequence⁶¹.

Therefore, we generated the CHO-K1 cell line stably expressing Gqi5, had it express the OPR, and then observed the cytoplasmic Ca²⁺ mobilization upon agonist addition, using the Ca²⁺-sensitive dyes (Supplementary FIG. 2-s1d; FIG. 2-6a-c). This method of using Gqi5 has been widely employed in the research of OPRs⁵⁰ and other Gi-coupled GPCRs^{51,52}.

Briefly, CHO-K1 cells stably expressing Gqi5 (Gαq with the C-terminal five amino-acid sequence [ECGLY] replaced by that of Gαi2 [DCGLF]) were generated and transfected with the cDNAs encoding the wild-type and SNAPf-linked OPRs. The Ca2+-sensitive dyes Fluo4-AM (Dojindo) and Rhod3-AM (Thermo Fisher Scientific) were employed. Rhod3 was used for the experiments with mGFP-Xpeps and FAM-Xpep-TATs, and Fluo4 was used for the experiments that did not employ these homodimer-blocking reagents. These AM dyes were incorporated in the cell, according to the manufacturers' recommendations, using the following solutions: 4.6 μM Fluo4-AM in T-HBSS containing 1.25 mM Probenecid (Dojindo) and 0.04% (w/v) Pluronic F127 (Dojindo), and 10 μM Rhod3-AM in T-HBSS containing 2.5 mM Probenecid and 1× PowerLoad™ (Thermo Fisher Scientific). These loading solutions (2 ml) were added to the cells and incubated in the dark at 37° C. for 30 min, and then the cells were washed three times with T-HBSS.

For the observations of the Ca²⁺ mobilization downstream of the SNAPf-tagged OPRs, we selected cells expressing SNAPf-tagged OPRs bound by SNAP-Surface 549 (or SNAP-CF660R for experiments using Rhod3) at number densities of 0.5-1.5 fluorescent spots/μm² in the basal PM, using single-molecule detection (TIRF illumination) in the 561-nm channel (642-nm channel when we employed Rhod3). These cells were then observed by epifluorescence illumination using the 488-nm channel to monitor the Fluo4 signal (561-nm channel to observe the Rhod3 signal and 488-nm channel for mGFP-Xpeps and FAM-Xpep-TATs). Agonist stimulation was performed by adding the DMSO solutions of agonists at a final concentration of 200 nM. To determine the saturation levels of the fluorescence signal intensity at higher concentrations of Ca²⁺, 1 μM (final concentration) ionomycin (Wako) was added (which would increase the intracellular Ca²⁺ concentration to that outside the cells [1.3 mM]). The Fluo-4 and Rhod-3 image sequences were analyzed using the ImageJ software.

For the comparison of the functions of the SNAPf-OPRs with those of the wild-type OPRs, we hoped to compare the cells expressing the SNAPf-OPRs at the levels of 0.5-1.5 spots/μm² in the basal PM with the cells expressing the wild-type OPRs at levels comparable to or higher than those of SNAPf-OPRs, because the cells expressing the wild-type OPRs should serve as the positive controls. For this purpose, the expression levels of wild-type OPRs were monitored by using cells transfected with the cDNA linking the wild-type OPR sequence to the mCherry sequence via the self-cleavable 2A linker sequence (mCherry-2A-OPR). This way, mCherry is released from the OPR into the cytoplasm at the ER, and the wild-type OPR is then transported to the PM. The expression of wild-type OPR was detected by the presence of mCherry in the cytoplasm using the epi-illumination at 561 nm (sensitivities much lower than single-molecule imaging, showing the presence of rather high concentrations of mCherry), indicating the higher expression of the wild-type OPR.

### Quantifying the Internalization of SNAPf-OPRs

The SNAPf-OPR was imaged at the level of single molecules using TIRF microscopy. The signal intensity of each individual spot was measured, and the sum of their intensities was calculated. However, the SNAPf-OPR spots found in this manner should include those formed by the already internalized SNAPf-OPR molecules that were still located near the PM and detected by TIRF microscopy. These molecules were selectively detected by adding the membrane-impermeable fluorescence quencher, Mn³⁺-TSP (Frontier Scientific). Briefly, Mn³⁺-TSP was dissolved in T-HBSS containing 1% BSA to a final concentration of 10 mM, and this solution was added to the cells in Ham's F12 observation medium, to a final concentration of 3.3 mM Mn³⁺-TSP. The sum of the intensities of the individual fluorescence spots after the quencher addition was obtained, and subtracted from the sum of the signal intensities before the quencher addition.

### Software and Statistical Analysis

The microscope station that combined a single-molecule imaging system and a super-resolution confocal microscope was controlled by in-house LabVIEW2018-based software, and the single-molecule movie acquisitions were performed using the MCR software (Hamamatsu Photonics) for Windows. Superimposition of single-molecule imaging sequences obtained in two colors and tracking of the spots in the movies were performed using C++-based computer programs produced in-house, as described previously^{57,59,62,63}. The TIRF images of FAM-Xpep-TATs were processed and analyzed using Image J for Windows or MATLAB 2019a for Windows. Curve fitting was performed by OriginPro 2019b for Windows. Statistical analysis was performed by Welch's two-tailed t test, except for the colocalization duration data, which were analyzed by the Brunner-Munzel test, using OriginPro 2019b for Windows and RStudio 1.2.1335 for Windows. P values less than 0.05 were considered to be statistically significant. The simulation study was performed using the in-house software based on MATLAB 2019a for Windows.

### <Results>

### Experimental Strategies

### (1) Predicting the OPR Sites Responsible for Homodimerization Based on the Amino-Acid Homology

Most of the studies examining the class A GPCR homodimerization mechanisms envisaged the involvement of transmembrane (TM) domains, primarily because a dimerization mechanism common to all of the dimer-forming GPCRs was sought^{7,28,29}. This is understandable because one of the common characteristics of GPCRs could be, in addition to the presence of seven transmembrane domains and the coupling to G-proteins, that they continually form transient homodimers.

However, in the cases of the three classical OPRs, KOR, MOR, and DOR, the idea that the TM domains are mainly responsible for homodimerization is fraught with a substantial problem (although TM interactions might enhance homodimerization). Since their TM domains exhibit ˜75% amino-acid identity and ˜90% similarity (for rat OPRs; FIG. 2-s1a), if they were responsible for homodimerization, then they would cause heterodimerization at almost equal levels. However, MOR and KOR exhibited very limited heterodimerization^{15,30} (however see Ref. 31), whereas the KOR-DOR and DOR-MOR heteromers were found functionally important^{15,32-35}.

In contrast, the N-terminal extracellular domains (51-70 amino-acid sequences) are quite different among the three OPRs, and the C-terminal cytoplasmic domains (47-59 amino-acid sequences) are also almost entirely different, except for nine amino acids in the initial part of the C-terminal cytoplasmic domains, which are identical among the three OPRs (for rat; only five amino acids are identical in the last 38-50 C-terminal residues of KOR, MOR, and DOR). Furthermore, the C-terminal cytoplasmic 15-residue domain of DOR was already reported to be responsible for DOR homodimerization quite some time ago using a biochemical approach¹⁰. Therefore, in the present research, we hypothesized that the N- and/or C-terminal cytoplasmic domains contain key regions responsible for the homodimerization of the three classical OPRs.

### (2) Developing the Theories and Methods for Evaluating (A) Homodimer Dissociation (Dimer-Monomer) Equilibrium Constant K_{D} and (B) Homodimer Lifetime Based on Single-Molecule Imaging Data

In the literature, often the argument is whether OPRs (or GPCRs) form homodimers or not, but the fact of the matter is that the majorities of proteins form dimers and clusters at very high expression levels and the physiological expression levels would vary greatly from cell to cell. Therefore, we need to quantitatively evaluate the homodimer affinity, i.e., the homodimer dissociation (dimer-monomer) equilibrium constant K_{D}. Therefore, we developed a theory and a method for evaluating K_{D} from the PCCF of the OPR fluorescently-labeled in two colors.

Furthermore, we developed a theory based on the diffusion equation that predicts the distribution of colocalization durations. Previously, we (Ref. 23 and 24, also see 38) and others (Ref. 9, 19, 22, 26, and 27) depended on intuitive methods to obtain the dimer lifetimes from the optical colocalization data. However, due to the theory developed here, we now have a firm ground to obtain the dimer lifetime from the single-molecule colocalization data. The final result from the theory is simple: the distribution of the colocalization durations could be fitted by a sum of two exponential functions, under our experimental conditions, and the longer lifetime provides the dimer lifetime.

### All Three OPRs Continually Interconvert Between Transient Homodimers and Monomers

OPRs conjugated with the SNAPf tag protein at their N-termini (SNAPf-OPRs) were expressed in the PM of CHO-K1 cells (these molecules were functional as shown in FIG. 2s1b, c) and labeled with the fluorescent membrane-impermeable SNAP ligands⁹, SNAP-Surface 549 and SNAP-CF660R, with ≥70% efficiencies³⁷ (FIG. 2-s2). For the experiments examining the homodimerization of OPRs, we simultaneously labeled SNAPf-OPR (for example, SNAPf-KOR) with both SNAP-Surface 549 and SNAP-CF660R so that the number densities of the two probes on the PM could become similar, with spot densities between 0.25-0.75 spots/μm² for each color image, and then performed simultaneous dual-color single-molecule observations at normal video rate (30 Hz) at 37° C., using a home-built total internal reflection fluorescence (TIRF) microscope.

Virtually all of the SNAPf-OPR fluorescent spots exhibited diffusion in the PM, but in addition, they exhibited frequent temporary colocalization and co-diffusion, consistent with the transient homodimer formation found for other GPCRs (FIG. 2-1a). Although the colocalization distance of 200 nm employed here (Methods) is much larger than molecular scales (<10 nm), the colocalization analysis is still useful for detecting molecular interactions: Unassociated molecules may track together by chance over brief periods for short distances, but the probability of this occurring for multiple frames is small^{23,24}. Consequently, longer colocalization durations imply the presence of molecular interactions between the two molecules or homodimers, rather than incidental encounters (although molecular interactions are initiated by incidental encounters). Using a long distance such as 200 nm was necessary to detect true molecular binding with a probability larger than 99%, due to the limited precision of single-molecule localization when diffusing molecules were observed at video rate³⁸.

In the following, we refer to homo-colocalization as homodimerization, for a clear and concise presentation. We focused on the colocalization of the spots with different colors in the simultaneous two-color experiments, due to the case with which the obtained images are analyzed. For single-color imaging data, see FIG. 2-s3, which shows an example of repeated homodimerization of a KOR molecule with different partner KOR molecules, consistent with the observations made with other GPCRs^{23,24,26}.

The extent of OPR homodimerization was analyzed by the colocalization index based on the pair cross-correlation function (PCCF) of the SNAP-Surface 549 (green) and SNAP-CF660R (magenta) spots, as previously reported³⁹ (FIG. 1b, FIG. 2-s4a-c). KOR, MOR, and DOR exhibited significantly higher colocalization indices than the negative controls (green image overlaid by the 180°-rotated magenta image; see FIG. 2-s4b, FIG. 2-1c). Under the expression conditions of 0.5-1.5 OPR spots/μm² employed in this study, the colocalization index barely depended on the expression levels (FIG. 2-s5a-f). KOR exhibited a higher propensity for forming homodimers than MOR and DOR (FIG. 2-1c; the main results are summarized in Table D and their statistical test results are in Supplementary Table 1). A previous biochemical study found that DOR forms homodimers¹⁰, consistent with our result.

The homodimer dissociation (dimer-monomer) equilibrium constant K_{D} was evaluated from the PCCFs and the total number of spots in the SNAP-Surface 549 and SNAP-CF660R images. The K_{D}'s were 2.68±0.28, 7.31±0.76, and 7.91±0.26 copies/μm² for KOR, MOR, and DOR, respectively (at 37° C.; FIG. 2-s5c). These values are largely consistent with the K_{D}'s of β2AR and FPR (1.6 and 3.6 copies/μm², respectively), but the K_{D} for the MOR homodimer was ˜3.5× smaller than that reported previously, using the fluorescent antagonist analogs (27.43±11.75 copies/μm²)¹⁹.

Previous single-molecule examinations^{9,22} demonstrated that MOR and KOR tend to exist as monomers even at 20° C., when their expression levels were between 0.1 and 0.3 copies/μm², at apparent variance with our results. However, the homodimer fraction should depend on the copy number density of the protein in the PM, and at the number densities of 0.5-1.5 OPR spots/μm² employed in our analyses, it is clear that MOR and KOR form homodimers, consistent with the MOR data using the fluorescently-conjugated MOR antagonist¹⁹.

### The Homodimer Lifetimes of the Three OPRs are in the Time Scale of ˜130 ms

The durations of all colocalization events of the spots with different colors were measured (>1,500 events in ≥17 cells). We thereby obtained the distribution of colocalization durations (histogram; FIG. 2-1d). We developed a theory based on the diffusion equation that predicts the distribution of colocalization durations. Following the theory, the histogram was fitted by the sum of two exponential decay functions (shorter and longer decay time constants; τ₁ and τ₂, respectively). Meanwhile, the distribution of the incidental colocalization durations found by overlaying the green movie and the 180°-rotated magenta movie (FIG. 2-1d; FIG. 2-s4b) could be fitted by a single exponential function with a decay constant τ_{inci}. Since τ₁ is almost the same as τ_{inci} (FIG. 2-1d), then according to the theory, τ₂ provides the colocalization (homodimer) lifetime (after correction for the photobleaching lifetimes of the two fluorescent probes; FIG. 2-s6): 149±26, 118±12, and 125±15 ms for KOR, MOR, and DOR, respectively (FIG. 2-1d).

### The 9˜26 Amino-Acid Sequences in the C-Terminal Cytoplasmic Domains Play Critical Roles in OPR Homodimerization

Following our strategy described at the beginning of Results section, we first examined the involvement of the extracellular N-terminal domains in homodimerization, using the N-terminal deletion mutants (KOR(Δ1-53), MOR(Δ1-51), and DOR(Δ1-35)). These ranges were selected partly because the removal of the entire N-terminal extracellular domains blocked the OPR expression in the PM, probably due to the deletion of the endogenous signal-peptide sequences, and also because these deletion mutants had been used for the X-ray crystallography studies. In these studies, in addition to the deletion of the N-terminal domains, the C-terminal domains were further deleted and the T4-lysozyme sequence was inserted in the third intracellular loop or the specific nanobody was added, to enhance the crystallization⁴⁰⁻⁴³. These N-terminal deletion mutants all exhibited diffusion behaviors similar to those of the wild types (without immobilization or clustering) and virtually the same colocalization indices as those of the wild types (FIG. 2-1c). These results clearly indicated that the N-terminal extracellular domains are not responsible for homodimerization.

We next examined C-terminal mutants with systematically varied deletions (FIG. 2-2a, b). They hardly exhibited immobility or clustering, and their colocalization indices showed that the KOR's 365-380 amino-acid sequence, MOR's 358-382 sequence, and DOR's 357-372 sequence are critical for homodimerization (FIG. 2-2b; Supplementary Table 2). The DOR amino-acid sequence for homo-oligomerization agrees with the previous biochemical data¹⁰.

The distribution (histogram) of the colocalization durations for the deletion mutants could be fitted by a single exponential function, rather than the sum of two exponential functions, and the decay time constants were only slightly longer than the incidental colocalization lifetimes (FIG. 2-2c; Supplementary Table 3), supporting the colocalization index data; i.e., these deletion mutants rarely form homodimers. Meanwhile, the interactions of the transmembrane (TMs) domains have traditionally been considered to be important for GPCR dimerization^{7,28,29,44}. Since we could not totally block OPR homodimerization by the deletion of the C-terminal region, the TM interactions might also be involved in OPR homodimerization, although the C-terminal domains must be the predominant and critical interaction sites.

By introducing point mutations in these critical regions and their surrounding regions (FIG. 2a), we found that a few to several basic/acidic residues, as well as the proline residues, are important for the OPR homodimerization (for particular amino acids, see the colocalization indices shown in FIG. 2-2d; Supplementary Table 2), suggesting that the electrostatic interactions and the overall structure of the cytoplasmic C-terminal domain could be important for homodimerization. Indeed, the IUPred2 scores (energy-estimation-based predictions for ordered and disordered residues; http://iupred2a.elte.hu)⁴⁵ suggested that the C-terminal region could be intrinsically disordered, and the scores decreased with these point mutations (FIG. 2-s1d, e). This result suggests that, in addition to specific amino-acid interactions, the multiple weak interactions of the intrinsically disordered domains of the OPRs' C-terminal regions could facilitate the OPR dimerization, as found for the dimerization of transcription factor PU.1⁴⁶.

Peptides with the Amino Acid Sequences Responsible for Homodimerization Block Homodimer Formation

We then examined whether the peptides with the same amino-acid sequences as those of the deleted parts of the deletion mutants could block homodimerization. The use of these peptides was critical for unequivocally demonstrating that the specific amino-acid sequences in the C-terminal regions of KOR, MOR, and DOR are responsible for homodimerizations, because the deletion and point mutations we employed (FIG. 2-2) might have induced conformational changes of the true homodimer interaction sites, leading to the inhibition of homodimerization (which is also true for the DOR homo-oligomerization amino-acid sequence determined previously¹⁰. Furthermore, if we could develop such homodimerization blockers, they could be used as extremely useful tools to dissect the functions of OPR monomers and homodimers.

We employed two approaches. The first one employs the peptide with the same amino-acid sequences as those of the deleted parts of the deletion mutants, conjugated to the C-terminus of mGFP (named mGFP-Kpep, Mpep, and Dpep, collectively called mGFP-Xpeps, and the numbers in parentheses following mGFP-Xpeps indicate the amino-acid residue ranges in the wild-type OPRs). These peptide-based molecules were expressed in CHO-K1 cells stably expressing SNAPf-KOR, MOR, or DOR (wild-type), respectively (FIG. 2-3a, b), and their effects on the OPRs' homodimerization were inspected (FIG. 2-3c, d; Supplementary Table 4). The concentration of the cytoplasmic mGFP-Xpep was measured by confocal fluorescence microscopy, based on the calibration employing various concentrations of the purified mGFP protein dissolved in the Ham's F12 observation medium in the glass-base dish, observed at 5 μm above the glass surface (Methods; FIG. 2-s7a).

The OPR homo-colocalization index in the cells expressing mGFP-Xpep in the cytoplasm (the protein was diffusely and homogeneously distributed throughout the cytoplasm; FIG. 2-3b) exhibited a clear tendency to decrease with an increase of the specific mGFP-Xpep concentration in the cytoplasm from 0 to 7.8 UM for all three OPRs, consistent with the K_{D} values (2.7˜7.9 copies/μm²) (FIG. 2-3c; statistical data shown in FIG. 2-s8a). Meanwhile, the control mGFP-peptides (mGFP-Dpep for KOR and MOR and mGFP-Kpep for DOR) had no effect (FIG. 2-3c; FIG. 2-s8a). In addition, the comparison of the colocalization index without (0 μM) mGFP-Xpep with the mean colocalization index for the mGFP-Xpep concentrations in the range of 3.8-7.8 μM showed a significant reduction of the colocalization index in the presence of the mGFP-Xpep, but not the control mGFP-peptide, for all three OPRs (FIG. 2-3c; Table D). Consistently, the homodimer lifetimes were reduced to the levels comparable to those for the incidental colocalization or the C-terminal deletion mutants in cells expressing 3.8-7.8 μM mGFP-Xpeps (FIG. 2-3d; Table D).

As another approach, K-, M-, and D-peps were conjugated with the fluorescent dye 5-FAM at their N-termini for visualization and with the TAT sequence (YGRKKRRQRRR) with a G10 linker at their C-termini for membrane permeabilization (FAM-Xpep-TAT; Methods), and then added to the cells preincubated with 150 μM pyrenebutyrate for 5 min⁴⁷ (FIG. 2-4a, b). The FAM-peptide-TAT exhibited a diffuse distribution throughout the cytosol (FIG. 2-4b). The cells containing 2.9-3.4 AM FAM-Xpep-TAT (see FIG. 2-s7b for the concentration calibration; in the following, instead of repeating 2.9-3.4 μM, we describe this concentration range as ˜3 μM for conciseness) were selected and the effects of these cytoplasmic peptides on the OPR homodimerization were examined. The results indicated that ˜3 μM FAM-peptide-TAT significantly blocked the homo-dimerization (FIG. 2-4c; statistical data shown in FIG. 2-s8b; Table D), consistent with the data obtained using mGFP-Xpeps (FIG. 2-3c). The control FAM-Xpep-TATs (FAM-Dpep-TAT for KOR and MOR and FAM-Kpep-TAT for DOR) did not affect the colocalization indices.

The presence of ˜3 μM FAM-Xpep-TATs in the cytosol greatly reduced the homodimer lifetimes to the levels of mutants with deletions of the dimer-inducing domains (FIG. 2-4d; Table D). These results are consistent with those obtained by expressing mGFP-Xpeps in the cell.

### Agonists Modulate the Monomer-Dimer Interconversions, and the FAM-Xpep-TAT Peptides Block the Modulation

The addition of representative agonists, U-50488 for KOR, [D-Ala², N-Mc-Phet, Gly⁵-ol]-enkephalin acetate salt (DAMGO) for MOR, and SNC-80 for DOR, at 0.2 μM (a concentration sufficient to ligate virtually all OPR molecules) affected homodimerizations of the three OPRs differently (within 5 min after the addition). The agonist-bound KOR exhibited less homodimerization (lower colocalization index), whereas the agonist-bound MOR and DOR exhibited more homodimerization (FIG. 2-4c; Table D), although these results could vary depending on the particular agonists^{9,10}. For example, Devi's group previously found that the DOR agonists, DADLE, DSLET, and DPDPE, induce less homodimers¹⁰, but we found that the DOR agonist, SNC-80, increases the homodimer fraction, suggesting that the dimerization propensity depends on each agonist. We only examined one agonist for each OPR subtype, because an extensive examination of the agonist effects on OPR homodimerization is beyond the scope of the present work.

Furthermore, the effects of the agonists on the homodimer lifetimes were consistent with the results of the colocalization indices. The homodimer lifetime of the agonist-bound KOR became shorter, whereas those of the agonist-bound MOR and DOR became longer (FIG. 2-4d).

In the presence of FAM-Xpep-TAT, the colocalization indices of the agonist-bound OPRs were reduced for all OPRs, but in different ways depending on the OPR subtype. The colocalization index of the agonist-bound KOR, which exhibited a smaller index as compared with that of non-ligated KOR, was further reduced by the presence of FAM-Kpep-TAT. The colocalization index of the agonist-bound MOR was reduced in the presence of FAM-Mpep-TAT to the level of the deletion mutant MOR (A358-382) (FIG. 2-2b). Meanwhile, the colocalization index of the agonist-bound DOR was reduced in the presence of FAM-Dpep-TAT, but it was still greater than that of the wild-type DOR in the absence of the agonist.

The effects of the presence of FAM-Xpep-TAT on the homodimer lifetimes of the agonist-bound DORs were largely consistent with the effects on the colocalization indices. The homodimer lifetime of the agonist-bound KOR was already comparable to that of the deletion mutant KOR (4365-380), and no further reduction by FAM-Kpep-TAT was observed. The homodimer lifetime of the agonist-bound MOR was reduced in the presence of FAM-Mpep-TAT to a level similar to that of the deletion mutant MOR (A358-382) (FIG. 2-2b). The homodimer lifetime of the agonist-bound DOR was reduced by the presence of FAM-Dpep-TAT, but was ˜1.5× longer than that of the wild-type DOR in the absence of the agonist.

### The Three Basic Constants to Describe the Monomer-Dimer Equilibrium for all Three OPRs

The dimer dissociation equilibrium constants (K_{D}) after agonist binding were evaluated, as done for the results before agonist stimulation (FIG. 2-4f). These values support the results of the direct readout of the colocalization indices, described in the previous subsection.

The inverse of the dimer lifetimes (FIGS. 2-1d, 2-4d) provides the dimer dissociation rate constants (k_{off}). k_{off} values before and after the agonist binding are summarized in FIG. 2-4f. Using experimentally evaluated k_{off} and K_{D}, the dimer formation rate constants kₒₙ were calculated, providing all the three basic constants to describe the monomer-dimer equilibrium for all three OPRs (FIG. 2-4f; see Supplementary Table 5 for SEMs). These basic constants unequivocally show the presence of homodimers for all three OPRs.

The ratios of molecules that exist as dimers vs. monomers (D/M ratios) in terms of the number of protomers were calculated from K_{D} at various expression levels (number density of molecules) (FIG. 2-4e, f). Their overall variations, including agonist-bound and unbound conditions, are in the range of 0.06˜2.28 (5.7˜70.0% of protomers in dimers) in the OPRs' predicted physiological expression ranges¹⁸⁻²⁰ of 0.3-10 copies/μm². Therefore, substantial amounts of dimers are expected to exist in various tissues at any time (the expected average copy numbers of monomers and dimers per cell at various expression levels are shown in Supplementary Table 6), but importantly, all of these dimers are forming and dispersing all the time, with the lifetimes of less than 0.3 s. When they dissociate into monomers, they will again form dimers, but the rate of dimer formation depends on the number density of the monomers (and thus the expression levels).

Effects of homodimer-blocking peptides on OPR internalization: OPR monomers and homodimers are internalized at equal rates both before and after agonist addition OPR internalization was monitored by using the membrane-impermeable fluorescence quencher, Mn(III) meso-tetra(4-sulfonatophenyl)porphine (Mn³⁺-TSP). This quencher only suppresses fluorescence emission from the SNAP-Surface 549 dye on the SNAPf-OPR in the PM, but not that in the cytoplasm. Accordingly, by subtracting the signal intensity after the quencher addition from that before the addition, we evaluated the percentages of the OPR molecules remaining in the PM (FIG. 2-s9; FIG. 2-5a). The time courses of the OPR numbers remaining in the PM after time 0 were examined in both the presence and absence of 0.2 μM agonists and ˜3 μM FAM-Xpep-TATs in the cytoplasm (FIG. 2-5b). The observations were made every 5 min (each observation was done for a single frame; i.e., 33 ms, and so the photobleaching of the fluorescent probe is negligible: the photobleaching lifetime will be [16.3±1.2]×[300/0.033] s or ˜41 h). Since we employed the OPR expression of ˜1 copy/μm², according to FIG. 2-4f, 33, 18, and 17% (18, 15, 29%) of KOR, MOR, and DOR copies would exist as dimers before (after) the agonist stimulation, and most of OPR molecules would be monomers in the presence of FAM-Xpep-TATs.

The time course of the OPR internalization could be operationally fitted by a single exponential function (FIG. 2-5b), providing the fraction of OPRs with detectable internalization by 35-min observations and their residency lifetimes in the PM as well as the fraction of molecules with much longer residency times, which cannot be measured in the present experimental design. In addition to our standard cell line, CHO-K1 cells, T24 cells were employed because the expression level of β2-arrestin, which might be involved in OPR internalization⁴⁸, was reportedly higher in T24 cells than that in CHO-K1 cells⁴⁹ (Table D).

The three OPRs all exhibited molecular fractions with longer unmeasurable residency times in the range of 91-97%, whereas the shorter measurable residency times (for about 3-9% of molecules) were between 24 and 45 min. The homodimer-blocking peptides, FAM-Xpep-TATs, did not significantly affect the OPR internalization, indicating that OPR internalization rates were the same whether they form monomers and homodimers (FIG. 2-5b; Table D; Supplementary Tables 7 and 8). In the absence of FAM-Xpep-TATs, the percentages of the OPR molecules existing as dimers are between 15 and 33%, but given the precisions of these measurements, if the internalization rates of monomers and dimers had been different by a factor of at least 2, we should have been able to detect the differences.

The agonist addition greatly increased the fractions of detectable internalizations and the internalization rates for all three OPRs, but the effects were smaller for MOR (agonists were U-50488, DAMGO, and SNC-80 for KOR, MOR, and DOR, respectively). The homodimerization-blocking peptides (both FAM-Xpep-TATs and mGFP-Xpeps) hardly affected engaged OPR internalizations, suggesting that the internalization rates of engaged OPRs were the same whether they form monomers and homodimers (FIG. 2-5b; Table D; Supplementary Tables 7 and 8).

### Homodimer-Blocking Peptides Modulate Agonist-Induced Signals in Various Ways

The agonist-induced signal downstream of the OPR was examined by a widely used method for Gi-coupled GPCR, employing an artificial G protein Gqi550-52. For the method, see the caption to FIG. 2-s1b and Methods (FIG. 2-6). We employed the cells expressing OPRs at ˜1 copy/μm², and thus again the percentages of OPR copies existing as dimers are in the range of 15˜33% in the absence of FAM-Xpep-TATs.

The addition of the homodimer-blocking FAM-peptide-TATs alone (thus the monomerization of OPRs alone) did not induce any detectable Ca²⁺ mobilization (FIG. 2-6d; Table D). Meanwhile, the addition of the respective agonists triggered Ca²⁺ mobilization. The effects of the homodimer-blocking peptides on the agonist-induced Ca²⁺ mobilization were complex (both FAM-Xpep-TATs and mGFP-Xpeps produced similar effects for all three OPRs). The homodimer-blocking peptides (OPR monomerization) enhanced, did not affect, and reduced the agonist-induced Ca²⁺ mobilization in cells expressing KOR, MOR, and DOR, respectively (FIG. 2-6d; Table D). These results indicate that KOR and DOR monomers trigger higher and lower signals than their respective homodimers (without influencing the agonist-induced internalization FIG. 2-5b), whereas MOR monomers and homodimers induce the downstream signals at similar levels. This result suggests that the homodimer-blocking peptide-TATs could be used as drugs to enhance or suppress the agonist-induced cellular responses for KOR and DOR, respectively, without affecting their internalization (see FIG. 2-5b).

TABLE 5
Table D. Summary of the results of homodimer experiments

| | | | Homodimer | Fraction of | Residency time | |
|---|---|---|---|---|---|---|
| | | Colocalization | Lifetime | internalized | of internalized | |
| | | Index | (Mean ± SEM) | molecules | molecules | Ca²⁺ |
| OPRs | Additions | (Mean ± SEM) | (ms) | (%) | (min) | (F_{Max}-F_{b})/ F_{b} |
| KOR | Control | 2.30 ± 0.2 | 149 ± 26 | 8.4 ± 4.1 | 32.1 ± 23.8 | 0.04 ± 0.01 |
| | + FAM-pep-TAT | 1.53 ± 0.1*^{,Na} | 68 ± 02*^{,Na} | 6.9 ± 2.4^{Nc,†} | 26.2 ± 15.4^{Nc,†} | 0.05 ± 0.01^{Nc,†} |
| | + mGFP-pep | 1.41 ± 0.1*^{,Na} | 60 ± 01*^{,Na} | — | — | — |
| | + Agonist | 1.48 ± 0.1* | 56 ± 01* | 51.4 ± 2.7* | 11.9 ± 1.7* | 1.96 ± 0.32* |
| | + Agonist | 1.23 ± 0.1*^{,†} | 54 ± 02*^{,Na} | 60.5 ± 2.5*^{,†} | 9.6 ± 1.2*^{,Na} | 2.46 ± 0.21*^{,†} |
| | + FAM-pep-TAT | | | | | |
| | + Agonist | 1.17 ± 0.1*^{,†} | 50 ± 02*^{,Na} | — | — | 3.11 ± 0.17*^{,†} |
| | + mGFP-pep-TAT | | | | | |
| MOR | Control | 1.82 ± 0.1 | 118 ± 12 | 6.4 ± 1.9 | 25.8 ± 12.6 | 0.03 ± 0.01 |
| | + FAM-pep-TAT | 1.38 ± 0.1*^{,†} | 66 ± 03*^{,†} | 6.3 ± 1.0^{Nc,†} | 22.1 ± 4.1^{Nc,†} | 0.04 ± 0.01^{Nc,†} |
| | + mGFP-pep | 1.32 ± 0.1*^{,†} | 57 ± 02*^{,†} | — | — | — |
| | + Agonist | 2.18 ± 0.2* | 187 ± 59* | 28.1 ± 1.7* | 11.3 ± 1.9 | 2.80 ± 0.21* |
| | + Agonist | 1.38 ± 0.1*^{,†} | 73 ± 03*^{,†} | 28.9 ± 2.0*^{,Na} | 10.7 ± 2.1*^{,Na} | 2.78 ± 0.22*^{,Na} |
| | + FAM-pep-TAT | | | | | |
| | + Agonist | 1.34 ± 0.1*^{,†} | 67 ± 03*^{,†} | — | — | 2.80 ± 0.24*Na |
| | + mGFP-pep | | | | | |
| DOR | Control | 1.89 ± 0.1 | 125 ± 15 | 4.5 ± 3.5 | 29.8 ± 37.4 | 0.05 ± 0.01 |
| | + FAM-pep-TAT | 1.36 ± 0.1*^{,†} | 73 ± 05*^{,†} | 4.9 ± 2.5^{Nc,†} | 26.2 ± 22.6^{Nc,†} | 0.05 ± 0.01^{Nc,†} |
| | + mGFP-pep | 1.29 ± 0.1*^{,†} | 64 ± 02*^{,†} | — | — | — |
| | + Agonist | 3.00 ± 0.3* | 276 ± 19* | 61.6 ± 1.7* | 10.8 ± 0.9* | 3.85 ± 0.23* |
| | + Agonist | 2.38 ± 0.2*^{,†} | 187 ± 32*^{,†} | 55.0 ± 1.0*^{,†} | 10.1 ± 0.5*^{,Na} | 2.84 ± 0.20*^{,†} |
| | + FAM-pep-TAT | | | | | |
| | + Agonist | 2.22 ± 0.2*^{,†} | 154 ± 26^{Nc,†} | — | — | 2.65 ± 0.22*^{,†} |
| | + mGFP-pep | | | | | |

*(Nc): Significant (insignificant) difference from the value for “Control”.
^{†}(Nc): Significant (insignificant) difference from the value for “+ Agonist”.
Table D. Summary of the colonization indices, homodifier lifetimes, the fraction of molecules whose internalizations were detectable by the present method, residency times in the PM, and the Ca²⁺ mobilization parameter, (F_{Max}-F_{b})/F_{b}, in the presence and absence of various modulators (0.2 μM agonists, 3 μM FAM-Xpep-TATs in the cytoplasm, and ~6 μM mGFP-Xpeps in the cytoplasm). The agonists employed here were U-50488 for KOR, DAMGO for MOR, and SNC-80 for DOR.

### DISCUSSION

By evaluating the K_{D}s and lifetimes of OPR homodimers based on single-molecule imaging-tracking data, we unequivocally demonstrated that all three OPRs form transient homodimers even at lower expression levels. The obtained K_{D} values, 2.68±0.28, 7.31±0.76, and 7.91±0.26 copies/μm² for KOR, MOR, and DOR, respectively (FIG. 2-s5b-d, f and g), are generally consistent with the previously obtained values for GPCRs (1.6 and 3.6 copies/μm² for β2AR and formyl-peptide receptor (FPR), respectively^{23,25}. Meanwhile, the DAMGO-induced enhancement of homodimerization reported previously⁹ was confirmed in the present study. Since we used only one agonist for each OPR subtype, further examinations using other agonists will be important.

Assuming the OPRs' predicted physiological expression ranges¹⁸⁻²⁰ of 0.3-10 copies/μm², for the overall variations of K_{D}, including agonist-bound and unbound conditions, 5.7˜70.0% of protomers would exist as dimers at any time in various tissues (FIG. 2-4c, f). Importantly, all of these dimers are forming and dispersing all the time, with the lifetimes of less than 0.3 s (FIG. 2-1d) at 37° C. When the homodimers dissociate into monomers, they will again form homodimers with the same and other partner molecules. Longer homodimer lifetimes on the order of 0.5˜1 s have been reported, but they were observed at lower temperatures such as RT and 20° C.^{9,26,27}.

From the sequence comparisons of the three classical OPRs and the likely assumption that the three OPRs form distinct homodimers and heterodimers^{10,15,30,33,34,35} we reasoned that the amino-acid sequences responsible for the homodimerization are those with lower amino-acid sequence identity and homology. Based on this presumption, we found that the specific amino acid sequences of 9˜26 residues in the near-C-terminal cytoplasmic domains are largely responsible for the distinct homodimerizations of all three classical OPRs (FIGS. 2-2b, c and 2-3). The TM domains might be involved in homodimerization, as suggested as the “rolling interface” model previously^{7,28,29}, and so it is possible that the TM domain interactions might be modulated by the interaction in the C-terminus cytoplasmic domains. The N-terminal extracellular domains are not involved in the homodimerization of any of the three OPRs (FIG. 2-1c).

In addition to charged amino acid residues, prolines appeared to be involved in homo-dimerization (FIG. 2-2d). Furthermore, all three OPRs exhibited IUPred2 scores close to or greater than 0.5 (FIG. 2-s1d, e), suggesting the intrinsically-disordered-domain-like conformations/properties of the C-terminal regions. Point mutations of the residues involved in homo-dimerization reduced the IUPred2 scores (FIG. 2-s1e). Therefore, for inducing homodimerization via the interaction of the cytoplasmic C-terminal domains, many weak interactions, in addition to localized electrostatic interactions, might be important.

By developing mGFP-Xpeps (FIG. 2-3) and FAM-Xpep-TATs (FIG. 2-4), we confirmed that the 9˜26 amino-acid sequences near the C-terminal cytoplasmic domains, without sequence similarities, are responsible for all three OPRs. To the best of our knowledge, these Xpeps were the first peptide-based drugs/reagents that could block GPCR homodimerization.

These homodimer-blocking peptides/proteins became important tools to dissect the functions of OPR monomers and homodimers. Somewhat surprisingly, the Xpep-induced blocking of homodimerization failed to affect the OPR internalization (desensitization) either before or after the agonist addition (FIG. 2-5b), indicating that OPR internalization rates were the same whether they form monomers and homodimers. Meanwhile, the use of the Xpeps revealed that DOR and KOR homodimers, but not MOR homodimers, signal differently from their respective monomers (FIG. 2-6d). This result implies that the interaction mechanisms of the receptor and G protein and/or the competition between G protein and GPCR-kinase/β-arrestin on the receptor might be quite different among the three OPRs, in the ways that involve the interfaces of homodimers⁵³.

Since the Xpep-TATs are membrane permeable, they can be used as drugs to modulate the downstream signals by enhancing the OPR monomer population. Therefore, the results reported here suggest a novel GPCR drug development strategy for regulating the downstream GPCR signals by modulating homodimer formation.

TABLE 6
Supplementary Table 1. P values for the results shown in Table 1, for determining the
statistical significance of the differences from the “Control” and “+Agonist”
data (indicated by the superscripted c and a before each number, respectively) using
Welch's two-tailed t test (except those for homodimer lifetimes, which were obtained
with the Brunner-Munzel test), as well as the number of examined cells (in parentheses).

| | | | | | Residency time | | |
|---|---|---|---|---|---|---|---|
| | | Colocalization | Homodimer | | of internalized | Ca²⁺ | |
| | | Index | Lifetime | | molecules (min) | (F_{Max} − F_{b})/Fb | |
| OPRs | Additions | (18 cells) | (# of cells) | | (10 cells) | (# of cells) | |
| KOR | Control | — | — | (18) | — | — | (18) |
| | | ^{c}1.4 × 10⁻⁴ | ^{c}<2.2 × 10⁻¹⁶ | (18) | ^{c}9.2 × 10⁻¹ | ^{c}7.6 × 10⁻¹ | (24) |
| | +FAM-pep-TAT | ^{a}7.9 × 10⁻¹ | ^{a}5.7 × 10⁻¹ | (18) | ^{a}1.2 × 10⁻³ | ^{a}2.4 × 10⁻⁹ | (24) |
| | | ^{c}1.1 × 10⁻⁵ | ^{c}<2.2 × 10⁻¹⁶ | (18) | | | |
| | +mGFP-pep | ^{a}9.7 × 10⁻¹ | ^{a}5.4 × 10⁻¹ | (18) | — | — | |
| | +Agonist | ^{c}9.8 × 10⁻⁵ | ^{c}<2.2 × 10⁻¹⁶ | (18) | ^{c}1.2 × 10⁻³ | ^{a}2.4 × 10⁻⁹ | (24) |
| | +Agonist | ^{c}2.0 × 10⁻⁶ | ^{c}<2.2 × 10⁻¹⁶ | (19) | ^{c}8.8 × 10⁻⁴ | ^{c}1.6 × 10⁻¹² | (20) |
| | +FAM-pep-TAT | ^{a}2.5 × 10⁻³ | ^{a}9.7 × 10⁻¹ | (19) | ^{a}3.6 × 10⁻¹ | ^{a}3.9 × 10⁻⁴ | (20) |
| | +Agonist | ^{c}1.3 × 10⁻⁶ | ^{c}<2.2 × 10⁻¹⁶ | (18) | | ^{c}1.7 × 10⁻¹⁰ | (22) |
| | +mGFP-pep | ^{a}1.2 × 10⁻³ | ^{a}2.1 × 10⁻¹ | (18) | — | ^{a}2.4 × 10⁻⁴ | (22) |
| MOR | Control | — | — | (17) | — | — | (20) |
| | | ^{c}2.4 × 10⁻⁵ | ^{c}5.2 × 10⁻¹² | (20) | ^{c}7.1 × 10⁻¹ | ^{c}3.3 × 10⁻¹ | (27) |
| | +FAM-pep-TAT | ^{a}3.5 × 10⁻⁵ | ^{a}<2.2 × 10⁻¹⁶ | (20) | ^{a}1.8 × 10⁻³ | ^{a}1.2 × 10⁻¹³ | (27) |
| | | ^{c}1.1 × 10⁻⁵ | ^{c}2.6 × 10⁻¹¹ | (20) | | | |
| | +mGFP-pep | ^{a}1.7 × 10⁻⁵ | ^{a}<2.2 × 10⁻¹⁶ | (20) | — | — | |
| | +Agonist | ^{c}5.8 × 10⁻² | ^{c}<2.2 × 10⁻¹⁶ | (20) | ^{c}1.5 × 10⁻³ | ^{a}1.2 × 10⁻¹³ | (29) |
| | +Agonist | ^{c}1.3 × 10⁻³ | ^{c}<2.2 × 10⁻¹⁶ | (20) | ^{c}2.1 × 10⁻⁴ | ^{c}1.7 × 10⁻¹⁵ | (22) |
| | +FAM-pep-TAT | ^{c}2.9 × 10⁻⁴ | ^{a}<2.2 × 10⁻¹⁶ | (20) | ^{a}8.6 × 10⁻¹ | ^{a}9.6 × 10⁻¹ | (22) |
| | +Agonist | ^{c}8.1 × 10⁻⁴ | ^{a}<2.2 × 10⁻¹⁶ | (18) | | ^{c}1.7 × 10⁻¹¹ | (20) |
| | +mGFP-pep | ^{a}1.8 × 10⁻⁴ | ^{a}<2.2 × 10⁻¹⁶ | (18) | | ^{a}9.2 × 10⁻¹ | (20) |
| DOR | Control | — | — | (18) | — | — | (18) |
| | | ^{c}3.6 × 10⁻⁶ | ^{c}1.1 × 10⁻³ | (18) | ^{c}6.3 × 10⁻¹ | ^{c}2.9 × 10⁻¹ | (22) |
| | +FAM-pep-TAT | ^{a}7.3 × 10⁻⁵ | ^{a}4.8 × 10⁻¹⁴ | (18) | ^{a}9.3 × 10⁻⁴ | ^{a}1.4 × 10⁻¹⁰ | (22) |
| | | ^{c}4.7 × 10⁻⁵ | ^{c}2.9 × 10⁻³ | (18) | | | |
| | +mGFP-pep | ^{a}2.8 × 10⁻⁵ | ^{c}6.7 × 10⁻¹⁵ | (18) | — | — | |
| | +Agonist | ^{c}3.9 × 10⁻³ | ^{c}<2.2 × 10⁻¹⁶ | (18) | ^{c}8.9 × 10⁻⁴ | ^{a}1.4 × 10⁻¹⁰ | (20) |
| | +Agonist | ^{c}8.4 × 10⁻² | ^{c}<2.2 × 10⁻¹⁶ | (18) | ^{c}8.8 × 10⁻⁴ | ^{c}1.0 × 10⁻¹⁵ | (24) |
| | +FAM-pep-TAT | ^{a}4.2 × 10⁻² | ^{a}<2.2 × 10⁻¹⁶ | (18) | ^{a}7.1 × 10⁻¹ | ^{a}3.8 × 10⁻² | (24) |
| | +Agonist | ^{c}2.6 × 10⁻³ | ^{c}<2.2 × 10⁻¹⁶ | (18) | | ^{c}2.6 × 10⁻¹⁶ | (20) |
| | +mGFP-pep | ^{a}3.7 × 10⁻² | ^{a}<2.2 × 10⁻¹⁶ | (18) | — | ^{a}9.5 × 10⁻⁶ | (20) |

TABLE 7
Supplementary Table 2. P values for determining the statistical significance
of the differences in the colocalization indexes between the OPR mutants
and the wild-type OPRs, as well as those among the mutants, using Welch's
two-tailed t test. n = 18 cells (See FIG. 2-2b, d).

| KOR | | MOR | | DOR | |
|---|---|---|---|---|---|
| mutants | p value | mutants | p value | mutants | p value |
| Δ351-364 | 4.6 × 10⁻¹ | Δ358-398 | 1.9 × 10⁻³ | Δ339-372 | 1.9 × 10⁻⁶ |
| Δ359-380 | 3.1 × 10⁻⁶ | Δ358-382 | 9.2 × 10⁻⁴ | Δ357-372 | 9.7 × 10⁻⁷ |
| Δ365-380 | 2.5 × 10⁻⁴ | Δ371-382 | 2.4 × 10⁻² | Δ357-364 | 7.4 × 10⁻² |
| Δ365-371 | 2.2 × 10⁻³ | Δ383-398 | 5.7 × 10⁻¹ | Δ365-372 | 2.9 × 10⁻⁴ |
| Δ372-380 | 9.6 × 10⁻⁴ | | | D364 | 6.8 × 10⁻¹ |
| D366 D372 | 7.7 × 10⁻³ | | | V357S | 2.0 × 10⁻¹ |
| R371 K378 | 6.5 × 10⁻⁴ | | | R354 R356 | 4.0 × 10⁻² |
| D366 D372 | 3.3 × 10⁻⁵ | E359 E372 | 4.6 × 10⁻³ | R354 R356 | 5.4 × 10⁻⁴ |
| R371 K378 | | D381 P374S | | P362 P366 | |
| P367 P379 | 1.4 × 10⁻² | R365 R367 | 1.5 × 10⁻² | P362 D364 | 1.7 × 10⁻² |
| | | R371 R382 | | P366 | |
| | | H373 P374S | | | |
| D366 D372 | 4.6 × 10⁻⁶ | E359 E372 | 1.8 × 10⁻⁴ | R346S R347S | 3.6 × 10⁻⁶ |
| R371 K378 | | D381 R365 | | R349S R354 | |
| P367 P379 | | R367 R371 | | R356 P342S | |
| | | R382 H373 | | P348S P362 | |
| | | P374S | | P366 | |

| KOR (Δ372-380) | | MOR (Δ358-382) | | DOR (Δ365-372) | |
|---|---|---|---|---|---|
| mutants | p value | mutants | p value | mutants | p value |
| Δ359-380 | 5.5 × 10⁻¹ | Δ358-398 | 5.6 × 10⁻¹ | Δ339-372 | 2.1 × 10⁻² |
| Δ365-380 | 8.3 × 10⁻¹ | Δ371-382 | 6.8 × 10⁻¹ | Δ357-364 | 1.4 × 10⁻² |
| Δ365-371 | 3.0 × 10⁻¹ | Δ383-398 | 4.6 × 10⁻³ | Δ357-372 | 4.8 × 10⁻² |
| MOR (Δ358-398) | | MOR (Δ383-398) | | DOR (Δ357-372) | |
| mutants | p value | mutants | p value | mutants | p value |
| Δ371-382 | 3.9 × 10⁻¹ | Δ371-382 | 1.4 × 10⁻² | Δ339-372 | 4.7 × 10⁻¹ |
| | | Δ358-398 | 3.8 × 10⁻³ | Δ357-364 | 5.5 × 10⁻⁵ |

| | | | | DOR (R346S R347S R349S | |
|---|---|---|---|---|---|
| KOR (D366 D372 R371 | | DOR (R354 R356 | | R354 R356 P342S | |
| K378 P367 P379) | | P362 P366) | | P348S P362 P366) | |
| mutants | p value | mutants | p value | mutants | p value |
| D366 D372 | 3.3 × 10⁻¹ | R354 R356 | 1.9 × 10⁻¹ | R354 R356 | 1.3 × 10⁻² |
| R371 K378 | | | | | |
| P367 P379 | 2.0 × 10⁻² | | | R354 R356 | 1.3 × 10⁻² |
| | | | | P362 P366 | |

TABLE 8
Supplementary Table 3. Summary of the lifetimes of OPR homodimers (τ₂) (See
FIG. 2-2c), after correction for the photobleaching lifetimes of SNAP-Surface
549 (16.3 ± 1.2 s) and SNAP-CF660R (7.8 ± 0.6 s) (See Supplementary FIG. 2-s6).

| | | Homodimer lifetime | P value | Number of | Number of |
|---|---|---|---|---|---|
| | | (Mean ± SEM) | (Brunner- | examined | examined |
| OPRs | | (ms) | Munzel test) | events | cells |
| KOR | WT | 149 ± 26 | — | 1680 | 18 |
| | Δ365-380 | 57 ± 3* | <2.2 × 10⁻¹⁶ | 1150 | 18 |
| MOR | WT | 118 ± 12 | — | 1887 | 17 |
| | Δ358-382 | 54 ± 4* | <2.2 × 10⁻¹⁶ | 1757 | 17 |
| DOR | WT | 125 ± 15 | — | 1987 | 18 |
| | Δ357-372 | 54 ± 4* | <2.2 × 10⁻¹⁶ | 1650 | 18 |

*Significantly different from the homodimer lifetimes for WT molecules.

TABLE 9
Supplementary Table 4. Summary of the number
of examined homodime determine the OPR homodimer
lifetimes (See FIG. 2-3d, FIG. 2-4d).

| | | Number of |
|---|---|---|
| OPRs | Additions | examined homodimers |
| KOR | Control | 1680 |
| | +FAM-Kpep-TAT | 1380 |
| | +mGPF-Kpep | 2246 |
| | +Agonist | 1904 |
| | +Agonist | 2382 |
| | +FAM-Kpep-TAT | |
| | +Agonist | 2080 |
| | +mGPF-Kpep | |
| MOR | Control | 1887 |
| | +FAM-Mpep-TAT | 1844 |
| | +mGPF-Mpep | 1907 |
| | +Agonist | 3827 |
| | +Agonist | 1140 |
| | +FAM-Mpep-TAT | |
| | +Agonist | 1259 |
| | +mGPF-Mpep | |
| DOR | Control | 1987 |
| | +FAM-Dpep-TAT | 2603 |
| | +mGPF-Dpep | 1608 |
| | +Agonist | 3212 |
| | +Agonist | 1796 |
| | +FAM-Dpep-TAT | |
| | +Agonist | 1580 |
| | +mGPF-Dpep | |

TABLE 10
Supplementary Table 5. Summary of K_{D} (dimer dissociation constant = dimer-monomer equilibrium constant), k_{off} (dimer
dissociation rate constant), and kₒₙ (dimer formation rate constant) for three OPRs before and after the addition (3-5 min) of
agonists. The ratios of the numbers of protomers that exist as dimers vs. monomers at different expression levels in the PM were
calculated from the three dynamic equilibrium constants and listed. Physiological expression levels are indicated by pink
highlighting. The values are the same as those shown in FIG. 2-4f, but SEMs are additionally shown in this table.

| | | | | | D/M ratio (in terms of the protomers; Mean ± SEM) | | | | |
|---|---|---|---|---|---|---|---|---|---|
| | | K_{D} | k_{off} | kₒₙ | at different expression levels in the PM (copies/μm²) | | | | |
| OPRs | Agonist | (copies/μm²) | (s⁻¹) | (copies/μm²/s) | 0.3 | 1 | 3 | 10 | 30 |
| KOR | − | 2.68 ± 0.28 | 6.71 ± 1.17 | 2.50 ± 0.51 | 0.19 ± 0.33 | 0.50 ± 0.15 | 1.08 ± 0.12 | 2.28 ± 0.16 | 4.26 ± 0.25 |
| | + | 7.61 ± 0.67 | 17.86 ± 0.32 | 2.35 ± 0.21 | 0.07 ± 0.72 | 0.22 ± 0.25 | 0.52 ± 0.13 | 1.20 ± 0.11 | 2.35 ± 0.14 |
| MOR | − | 7.31 ± 0.76 | 8.47 ± 0.86 | 1.16 ± 0.17 | 0.08 ± 0.81 | 0.22 ± 0.28 | 0.54 ± 0.15 | 1.23 ± 0.13 | 2.41 ± 0.17 |
| | + | 9.77 ± 1.14 | 5.35 ± 1.69 | 0.55 ± 0.18 | 0.06 ± 1.20 | 0.17 ± 0.40 | 0.43 ± 0.19 | 1.02 ± 0.14 | 2.03 ± 0.17 |
| DOR | − | 7.91 ± 0.26 | 8.00 ± 0.96 | 1.01 ± 0.13 | 0.07 ± 0.28 | 0.21 ± 0.09 | 0.50 ± 0.05 | 1.17 ± 0.04 | 2.30 ± 0.05 |
| | + | 3.58 ± 0.18 | 3.62 ± 0.25 | 1.01 ± 0.09 | 0.15 ± 0.20 | 0.40 ± 0.08 | 0.89 ± 0.06 | 1.92 ± 0.07 | 3.62 ± 0.11 |

TABLE 11
Supplementary Table 6. The numbers of protomers that are expected to exist as monomers and
dimers in the PM at different expression levels (total numbers of molecules in the PM).
The number densities of OPRs were 0.3, 1, 3, and 10 copies/μm² (the same as
in FIG. 2-4f and Supplementary Table 5), and the cell is assumed
to have a disk shape with a 15.5 μm radius and no thickness (although this
is unrealistic for neurons), giving total numbers of expressed molecules
of 450, 1,500, 4,500, and 15,000 copies per cell, respectively.

| | | The number of protomers that exist as monomers and dimers in the PM (copies) at different expression levels | | | | | | | |
|---|---|---|---|---|---|---|---|---|---|
| | | 450 copies | | 1,500 copies | | 4,500 copies | | 15,000 copies | |
| OPRs | Agonist | Monomers | Dimers | Monomers | Dimers | Monomers | Dimers | Monomers | Dimers |
| KOR | − | 380 | 70 | 1,000 | 500 | 2,200 | 2,300 | 4,600 | 10,400 |
| | + | 420 | 30 | 1,200 | 300 | 3,000 | 1,500 | 6,800 | 8,200 |
| MOR | − | 420 | 30 | 1,200 | 300 | 2,900 | 1,600 | 6,700 | 8,300 |
| | + | 420 | 30 | 1,300 | 200 | 3,100 | 1,400 | 7,400 | 7,600 |
| DOR | − | 420 | 30 | 1,200 | 300 | 3,000 | 1500 | 6,900 | 8,100 |
| | + | 390 | 60 | 1,100 | 400 | 2,400 | 2,100 | 5,100 | 9,900 |

TABLE 12
Supplementary Table 7. Summary of the fractions of internalized OPRs,
detectable by the observation durations employed here, and their residency
lifetimes in the PM. CHO-K1 cells vs. T24 cells. The agonists were U-
50488 for KOR, DAMGO for MOR, and SNC-80 for DOR (see FIG. 2-5b).

| | | CHO-K1 | | T24 | |
|---|---|---|---|---|---|
| | | Fraction | Residency time | Fraction | Residency time |
| | | of internalized | of internalized | of internalized | of internalized |
| OPRS | Additions | molecules (%) | molecules (min) | molecules (%) | molecules (min) |
| KOR | Control | 8.4 ± 4.1 | 32.1 ± 23.8 | 3.9 ± 2.9 | 44.6 ± 46.1 |
| | +FAM-pep-TAT | 6.9 ± 2.4^{†} | 26.2 ± 15.4^{†} | 5.3 ± 4.2^{†} | 37.4 ± 42.8^{†} |
| | +Agonist | 51.4 ± 2.7* | 11.9 ± 1.7* | 70.3 ± 2.0* | 16.9 ± 1.0* |
| | +Agonist | 60.5 ± 2.5*^{†} | 9.6 ± 1.2* | 65.6 ± 2.5* | 11.1 ± 1.2* |
| | +FAM-pep-TAT | | | | |
| | +Agonist | — | — | 70.9 ± 1.0* | 11.5 ± 0.4* |
| | +mGFP-pep | | | | |
| MOR | Control | 6.4 ± 1.9 | 25.8 ± 12.6 | 4.2 ± 2.4 | 40.3 ± 31.8 |
| | +FAM-pep-TAT | 6.3 ± 1.0^{†} | 22.1 ± 4.1^{†} | 5.2 ± 2.6^{†} | 36.8 ± 26.3^{†} |
| | +Agonist | 28.1 ± 1.7* | 11.3 ± 1.9* | 26.3 ± 1.1* | 6.5 ± 1.2* |
| | +Agonist | 28.9 ± 2.0* | 10.7 ± 2.1* | 29.2 ± 1.9* | 14.4 ± 2.2* |
| | +FAM-pep-TAT | | | | |
| | +Agonist | — | — | 28.6 ± 2.0* | 10.6 ± 2.2* |
| | +mGFP-pep | | | | |
| DOR | Control | 4.5 ± 3.5 | 29.8 ± 37.4 | 2.9 ± 1.0 | 24.2 ± 5.6 |
| | +FAM-pep-TAT | 4.9 ± 2.5^{†} | 26.2 ± 22.6^{†} | 3.9 ± 1.0^{†} | 21.4 ± 9.6^{†} |
| | +Agonist | 61.6 ± 1.7* | 10.8 ± 0.9* | 56.5 ± 1.9* | 8.9 ± 1.0* |
| | +Agonist | 55.0 ± 1.0*^{†} | 10.1 ± 0.5* | 57.2 ± 2.9* | 10.5 ± 1.6* |
| | +FAM-pep-TAT | | | | |
| | +Agonist | — | — | 56.5 ± 1.6* | 9.9 ± 0.9* |
| | +mGFP-pep | | | | |

*Significantly different from the values for the control samples. The significance was examined for all applicable cases, and when the difference was non-significant, no indication is shown. For the p values, see Supplementary Table 8.
^{†}Significantly different from the values for the samples of “+Agonist”. The significance was examined for all applicable cases, and when the difference was non-significant, no indication is shown. For the p values, see Supplementary Table 8.

TABLE 13
Supplementary Table 8. P values for the results shown in Supplementary Table
7, for determining the statistical significance of the difference from the
“Control” and “+Agonist” data (indicated by the superscripted
c and a before each number, respectively) using Welch's two-tailed t test (n = 10 cells).

| | | CHO-K1 | | T24 | |
|---|---|---|---|---|---|
| | | Fraction | Residency time of | Fraction | Residency time of |
| | | of internalized | internalized | of internalized | internalized |
| OPRs | Additions | molecules (%) | molecules (min) | molecules (%) | molecules (min) |
| KOR | Control | — | — | — | — |
| | | ^{c}6.2 × 10⁻¹ | ^{c}9.2 × 10⁻¹ | ^{c}5.4 × 10⁻² | ^{c}2.2 × 10⁻¹ |
| | +FAM-pep-TAT | ^{a}<2.2 × 10⁻¹⁶ | ^{a}1.2 × 10⁻³ | ^{a}<2.2 × 10⁻¹⁶ | ^{a}1.8 × 10⁻³ |
| | +Agonist | ^{c}<2.2 × 10⁻¹⁶ | ^{c}1.2 × 10⁻³ | ^{c}<2.2 × 10⁻¹⁶ | ^{c}1.6 × 10⁻³ |
| | +Agonist | ^{c}<2.2 × 10⁻¹⁶ | ^{c}8.8 × 10⁻⁴ | ^{c}<2.2 × 10⁻¹⁶ | ^{c}8.4 × 10⁻⁴ |
| | +FAM-pep-TAT | ^{a}6.6 × 10⁻⁸ | ^{a}3.6 × 10⁻¹ | ^{a}8.7 × 10⁻² | ^{a}6.7 × 10⁻¹ |
| | +Agonist | | | ^{c}<2.2 × 10⁻¹⁶ | ^{c}9.3 × 10⁻⁴ |
| | +mGFP-pep | — | — | ^{a}1.5 × 10⁻¹ | ^{a}5.1 × 10⁻¹ |
| MOR | Control | — | — | — | — |
| | | ^{c}6.6 × 10⁻¹ | ^{c}7.1 × 10⁻¹ | ^{c}2.4 × 10⁻¹ | ^{c}3.9 × 10⁻¹ |
| | +FAM-pep-TAT | ^{a}3.7 × 10⁻¹⁶ | ^{a}1.8 × 10⁻³ | ^{a}<2.2 × 10⁻¹⁶ | ^{a}6.4 × 10⁻⁴ |
| | +Agonist | ^{c}<2.2 × 10⁻¹⁶ | ^{c}1.5 × 10⁻³ | ^{c}<2.2 × 10⁻¹⁶ | ^{c}5.7 × 10⁻⁴ |
| | +Agonist | ^{c}<2.2 × 10⁻¹⁶ | ^{c}2.1 × 10⁻⁴ | ^{c}<2.2 × 10⁻¹⁶ | ^{c}1.8 × 10⁻³ |
| | +FAM-pep-TAT | ^{a}2.2 × 10⁻¹ | ^{a}8.6 × 10⁻¹ | ^{a}1.5 × 10⁻¹ | ^{a}5.6 × 10⁻¹ |
| | +Agonist | | | ^{c}<2.2 × 10⁻¹⁶ | ^{c}9.1 × 10⁻⁴ |
| | +mGFP-pep | — | — | ^{a}7.9 × 10⁻¹ | ^{a}8.6 × 10⁻¹ |
| DOR | Control | — | — | — | — |
| | | ^{c}2.2 × 10⁻¹ | ^{c}6.3 × 10⁻¹ | ^{c}7.5 × 10⁻² | ^{c}8.1 × 10⁻¹ |
| | +FAM-pep-TAT | ^{a}<2.2 × 10⁻¹⁶ | ^{a}9.3 × 10⁻⁴ | ^{a}<2.2 × 10⁻¹⁶ | ^{a}7.3 × 10⁻⁴ |
| | +Agonist | ^{c}<2.2 × 10⁻¹⁶ | ^{c}8.9 × 10⁻⁴ | ^{c}<2.2 × 10⁻¹⁶ | ^{c}7.2 × 10⁻⁴ |
| | +Agonist | ^{c}<2.2 × 10⁻¹⁶ | ^{c}8.8 × 10⁻⁴ | ^{c}<2.2 × 10⁻¹⁶ | ^{c}8.3 × 10⁻⁴ |
| | +FAM-pep-TAT | ^{a}1.2 × 10⁻⁷ | ^{a}7.1 × 10⁻¹ | ^{a}4.1 × 10⁻¹ | ^{a}8.8 × 10⁻¹ |
| | +Agonist | | | ^{c}<2.2 × 10⁻¹⁶ | ^{c}8.7 × 10⁻⁴ |
| | +mGFP-pep | — | — | ^{a}9.6 × 10⁻¹ | ^{a}8.9 × 10⁻¹ |

### REFERENCES (HOMODIMER BLOCKER)

1. Darcq, E. & Kieffer, B. L. Opioid receptors: drivers to addiction? Nat. Rev. Neurosci. 19, 499-514 (2018).
2. Valentino, R. J. & Volkow, N. D. Untangling the complexity of opioid receptor function. Neuropsychopharmacology 43, 2514-2520 (2018).
3. Schmid, C. L. et al. Bias factor and therapeutic window correlate to predict safer opioid analgesics. Cell 171, 1165-1175.e1113 (2017).
4. Sungkaworn, T. et al. Single-molecule imaging reveals receptor—G protein interactions at cell surface hot spots. Nature 550, 543-547 (2017).
5. Rios, C. D., Jordan, B. A., Gomes, I. & Devi, L. A. G-protein-coupled receptor dimerization: modulation of receptor function. Pharmacol. Ther. 92, 71-87 (2001).
6. Terrillon, S. & Bouvier, M. Roles of G-protein-coupled receptor dimerization. EMBO reports 5, 30-34 (2004).
7. Ferre, S. et al. G protein-coupled receptor oligomerization revisited: functional and pharmacological perspectives. Pharmacol. Rev. 66, 413 (2014).
8. Milligan, G., Ward, R. J. & Marsango, S. GPCR homo-oligomerization. Curr. Opin. Cell Biol. 57, 40-47 (2019).
9. Moller, J. et al. Single-molecule analysis reveals agonist-specific dimer formation of μ-opioid receptors. Nat. Chem. Biol. 16, 946-954 (2020).
10. Cvejic, S. & Devi, L. A. Dimerization of the delta opioid receptor: implication for a role in receptor internalization. J. Biol. Chem. 272, 26959-26964 (1997).
11. George, S. R. et al. Oligomerization of mu- and delta-opioid receptors: Generation of novel functional properties. J. Biol. Chem. 275, 26128-26135 (2000).
12. McVey, M. et al. Monitoring receptor oligomerization using time-resolved fluorescence resonance energy transfer and bioluminescence resonance energy transfer: The human δ-opioid receptor displays constitutive oligomerization at the cell surface, which is not regulated by receptor occupancy. J. Biol. Chem. 276, 14092-14099 (2001).
13. He, L., Fong, J., von Zastrow, M. & Whistler, J. L. Regulation of opioid receptor trafficking and morphine tolerance by receptor oligomerization. Cell 108, 271-282 (2002).
14. Zheng, H. et al. Palmitoylation and membrane cholesterol stabilize μ-opioid receptor homodimerization and G protein coupling. BMC Cell Biol. 13, 6 (2012).
15. Jordan, B. A. & Devi, L. A. G-protein-coupled receptor heterodimerization modulates receptor function. Nature 399, 697-700 (1999).
16. Gomes, I., Filipovska, J., Jordan, B. A. & Devi, L. A. Oligomerization of opioid receptors. Methods 27, 358-365 (2002).
17. Wang, D., Sun, X., Bohn, L. M. & Sadee, W. Opioid receptor homo- and heterodimerization in living cells by quantitative bioluminescence resonance energy transfer. Mol. Pharmacol. 67, 2173-2184 (2005).
18. Meral, D. et al. Molecular details of dimerization kinetics reveal negligible populations of transient μ-opioid receptor homodimers at physiological concentrations. Sci. Rep. 8, 7705 (2018).
19. Gentzsch, C. et al. Selective and wash-resistant fluorescent dihydrocodeinone derivatives allow single-molecule imaging of μ-opioid receptor dimerization. Angew. Chem. Int. Ed. 59, 5958-5964 (2020).
20. Asher, W. B. et al. Single-molecule fret imaging of GPCR dimers in living cells. Nat. Methods 18, 397-405 (2021).
21. Cechova, K. et al. Kappa but not delta or mu opioid receptors form homodimers at low membrane densities. Cell. Mol. Life Sci. 78, 7557-7568 (2021).
22. Drakopoulos, A. et al. Investigation of inactive-state κ opioid receptor homodimerization via single-molecule microscopy using new antagonistic fluorescent probes. J. Med. Chem. 63, 3596-3609 (2020).
23. Kasai, R. S. et al. Full characterization of GPCR monomer-dimer dynamic equilibrium by single molecule imaging. J. Cell Biol. 192, 463-480 (2011).
24. Kasai, R. S. et al. The Class-A GPCR dopamine D2 receptor forms transient dimers stabilized by agonists: detection by single-molecule tracking. Cell Biochem. Biophys. 76, 29-37 (2018).
25. Kasai, R. S., Fujiwara, T. K. & Kusumi, A. Metastable GPCR dimers trigger the basal signal by recruiting G-proteins. bioRxiv, 2020.02.10.929588 (2020).
26. Hern, J. A. et al. Formation and dissociation of MI muscarinic receptor dimers seen by total internal reflection fluorescence imaging of single molecules. Proc. Natl. Acad. Sci. U.S.A. 107, 2693 (2010).
27. Calebiro, D. et al. Single-molecule analysis of fluorescently labeled G-protein-coupled receptors reveals complexes with distinct dynamics and organization. Proc. Natl. Acad. Sci. U.S.A. 110, 743 (2013).
28. Dijkman, P. M. et al. Dynamic tunable G protein-coupled receptor monomer-dimer populations. Nat. Commun. 9, 1710 (2018).
29. Johnston, J. M. et al. Making structural sense of dimerization interfaces of delta opioid receptor homodimers. Biochemistry 50, 1682-1690 (2011).
30. Filizola, M., Olmea, O. & Weinstein, H. Prediction of heterodimerization interfaces of G-protein coupled receptors with a new subtractive correlated mutation method. Protein Eng. Des. Sel. 15, 881-885 (2002).
31. Chakrabarti, S., Liu, N.-J. & Gintzler, A. R. Formation of μ-/κ-opioid receptor heterodimer is sex-dependent and mediates female-specific opioid analgesia. Proc. Natl. Acad. Sci. U.S.A. 107, 20115-20119 (2010).
32. Rozenfeld, R. & Devi, L. A. Receptor heterodimerization leads to a switch in signaling: β-arrestin2-mediated erk activation by μ-δ opioid receptor heterodimers. FASEB J. 21, 2455-2465 (2007).
33. Gomes, I. et al. A role for heterodimerization of μ and δ opiate receptors in enhancing morphine analgesia. Proc. Natl. Acad. Sci. U.S.A. 101, 5135 (2004).
34. Gomes, I. et al. Identification of a μ-δ opioid receptor heteromer-biased agonist with antinociceptive activity. Proc. Natl. Acad. Sci. U.S.A. 110, 12072 (2013).
35. Waldhoer, M. et al. A heterodimer-selective agonist shows in vivo relevance of G protein-coupled receptor dimers. Proc. Natl. Acad. Sci. U.S.A. 102, 9050 (2005).
36. Morise, J. et al. AMPA receptors in the synapse turnover by monomer diffusion. Nat. Commun. 10, 5245 (2019).
37. Tsunoyama, T. A. et al. Super-long single-molecule tracking reveals dynamic-anchorage-induced integrin function. Nat. Chem. Biol. 14, 497-506 (2018).
38. Koyama-Honda, I. et al. High-speed single-molecule imaging reveals signal transduction by induced transbilayer raft phases. J. Cell Biol. 219 (2020).
39. Stone, M. B. & Veatch, S. L. Steady-state cross-correlations for live two-colour super-resolution localization data sets. Nat. Commun. 6, 7347 (2015).
40. Wu, H. et al. Structure of the human κ-opioid receptor in complex with JDTic. Nature 485, 327-332 (2012).
41. Manglik, A. et al. Crystal structure of the μ-opioid receptor bound to a morphinan antagonist. Nature 485, 321-326 (2012).
42. Granier, S. et al. Structure of the δ-opioid receptor bound to naltrindole. Nature 485, 400-404 (2012).
43. Huang, W. et al. Structural insights into μ-opioid receptor activation. Nature 524, 315-321 (2015).
44. Filizola, M. & Devi, L. A. Structural biology: how opioid drugs bind to receptors. Nature 485, 314-317 (2012).
45. Meszaros, B., Erdos, G. & Dosztanyi, Z. IUPred2A: context-dependent prediction of protein disorder as a function of redox state and protein binding. Nucleic Acids Res. 46, W329-W337 (2018).
46. Xhani, S. et al. Intrinsic disorder controls two functionally distinct dimers of the master transcription factor PU.1. Sci. Adv. 6, eaay3178 (2020).
47. Inomata, K. et al. High-resolution multi-dimensional NMR spectroscopy of proteins in human cells. Nature 458, 106-109 (2009).
48. Gurevich, V. V. & Gurevich, E. V. GPCR signaling regulation: the role of GRKs and arrestins. Front. Pharmacol. 10 (2019).
49. Menard, L. et al. Synergistic regulation of beta2-adrenergic receptor sequestration: intracellular complement of beta-adrenergic receptor kinase and beta-arrestin determine kinetics of internalization. Mol. Pharmacol. 51 5, 800-808 (1997).
50. Camarda, V. & Calo', G. Chimeric G proteins in fluorimetric calcium assays: experience with opioid receptors. in Calcium Signaling Protocols (eds. Lambert, D. G. & Rainbow, R. D.) 293-306 (Humana Press, Totowa, N. J., 2013).
51. Yokoyama, T., Kato, N. & Yamada, N. Development of a high-throughput bioassay to screen melatonin receptor agonists using human melatonin receptor expressing CHO cells. Neurosci. Lett. 344, 45-48 (2003).
52. Han, Y. et al. Allosteric communication between protomers of dopamine class A GPCR dimers modulates activation. Nat. Chem. Biol. 5, 688-695 (2009).
53. Mahoney, J. P. & Sunahara, R. K. Mechanistic insights into GPCR-G protein interactions. Curr. Opin. Struct. Biol. 41, 247-254 (2016).
54. Schulz, R., Wehmeyer, A. & Schulz, K. Opioid receptor types selectively cointernalize with G protein-coupled receptor kinases 2 and 3. J. Pharmacol. Exp. Ther. 300, 376 (2002).
55. Shaner, N. C. et al. Improved monomeric red, orange and yellow fluorescent proteins derived from Discosoma sp. red fluorescent protein. Nat. Biotechnol. 22, 1567-1572 (2004).
56. Naruse, K., Sai, X., Yokoyama, N. & Sokabe, M. Uni-axial cyclic stretch induces c-src activation and translocation in human endothelial cells via SA channel activation. FEBS Lett. 441, 111-115 (1998).
57. Koyama-Honda, I. et al. Fluorescence imaging for monitoring the colocalization of two single molecules in living cells. Biophys. J. 88, 2126-2136 (2005).
58. Murakoshi, H. et al. Single-molecule imaging analysis of Ras activation in living cells. Proc. Natl. Acad. Sci. U.S.A. 101, 7317 (2004).
59. Fujiwara, T. K. et al. Confined diffusion of transmembrane proteins and lipids induced by the same actin meshwork lining the plasma membrane. Mol. Biol. Cell 27, 1101-1119 (2016).
60. Cowen, T., Haven, A. J. & Burnstock, G. Pontamine sky blue: a counterstain for background autofluorescence in fluorescence and immunofluorescence histochemistry. Histochemistry 82, 205-208 (1985).
61. Conklin, B. R. et al. Substitution of three amino acids switches receptor specificity of Gqα to that of Giα. Nature 363, 274-276 (1993).
62. Fujiwara, T., Ritchie, K., Murakoshi, H., Jacobson, K. & Kusumi, A. Phospholipids undergo hop diffusion in compartmentalized cell membrane. J. Cell Biol. 157, 1071-1082 (2002).
63. Komura, N. et al. Raft-based interactions of gangliosides with a GPI-anchored receptor. Nat. Chem. Biol. 12, 402-410 (2016).

### REFERENCES (HETERODIMER BLOCKER)

1. Schmid, C. L. et al. Bias factor and therapeutic window correlate to predict safer opioid analgesics. Cell 171, 1165-1175.e1113, doi:10.1016/j.cell.2017.10.035 (2017).
2. Sungkaworn, T. et al. Single-molecule imaging reveals receptor-G protein interactions at cell surface hot spots. Nature 550, 543-547, doi:10.1038/nature24264 (2017).
3. Darcq, E. & Kieffer, B. L. Opioid receptors: drivers to addiction? Nat. Rev. Neurosci. 19, 499-514, doi:10.1038/s41583-018-0028-x (2018).
4. Valentino, R. J. & Volkow, N. D. Untangling the complexity of opioid receptor function. Neuropsychopharmacology 43, 2514-2520, doi:10.1038/s41386-018-0225-3 (2018).
5. Ong, E. W. & Cahill, C. M. Molecular Perspectives for mu/delta Opioid Receptor Heteromers as Distinct, Functional Receptors. Cells 3, 152-179 (2014).
6. Gaborit, M. & Massotte, D. Therapeutic potential of opioid receptor heteromers in chronic pain and associated comorbidities. British Journal of Pharmacology n/a, doi:https://doi.org/10.1111/bph.15772.
7. George, S. R. et al. Oligomerization of mu- and delta-opioid receptors. Generation of novel functional properties. J. Biol. Chem. 275, 26128-26135, doi:10.1074/jbc.M000345200 (2000).
8. Gomes, I. et al. A role for heterodimerization of μ and δ opiate receptors in enhancing morphine analgesia. Proc. Natl. Acad. Sci. U.S.A. 101, 5135, doi:10.1073/pnas.0307601101 (2004).
9. Xie, W.-Y. et al. Disruption of Cdk5-associated phosphorylation of residue threonine-161 of the 8-Opioid Receptor: impaired receptor function and attenuated morphine antinociceptive tolerance. J. Neurosci. 29, 3551, doi:10.1523/JNEUROSCI.0415-09.2009 (2009).
10. Chefer, V. I. & Shippenberg, T. S. Augmentation of morphine-induced sensitization but reduction in morphine tolerance and reward in delta-opioid receptor knockout mice. Neuropsychopharmacology 34, 887-898, doi:10.1038/npp.2008.128 (2009).
11. Wang, D. et al. Functional divergence of delta and mu opioid receptor organization in CNS Pain Circuits. Neuron 98, 90-108.e105, doi:https://doi.org/10.1016/j.neuron.2018.03.002 (2018).
12. Jordan, B. A. & Devi, L. A. G-protein-coupled receptor heterodimerization modulates receptor function. Nature 399, 697-700, doi:10.1038/21441 (1999).
13. Waldhoer, M. et al. A heterodimer-selective agonist shows in vivo relevance of G protein-coupled receptor dimers. Proc. Natl. Acad. Sci. U.S.A. 102, 9050, doi:10.1073/pnas.0501112102 (2005).
14. Berg, K. A. et al. Allosteric interactions between δ and κ opioid receptors in peripheral sensory neurons. Mol. Pharmacol. 81, 264, doi:10.1124/mol.111.072702 (2012).
15. Jacobs, B. A. et al. Allosterism within δ opioid-κ Opioid Receptor heteromers in peripheral sensory neurons: regulation of κ opioid agonist efficacy. Mol. Pharmacol. 93, 376-386, doi:10.1124/mol.117.109975 (2018).
16. Jacobs, B. A. et al. Signaling characteristics and functional regulation of delta opioid-kappa opioid receptor (DOP-KOP) heteromers in peripheral sensory neurons. Neuropharmacology 151, 208-218, doi:https://doi.org/10.1016/j.neuropharm.2019.02.019 (2019).
17. Ansonoff, M. A., Portoghese, P. S. & Pintar, J. E. Consequences of opioid receptor mutation on actions of univalent and bivalent kappa and delta ligands. Psychopharmacology 210, 161-168, doi:10.1007/s00213-010-1826-7 (2010).
18. Gupta, A. et al. Increased abundance of opioid receptor heteromers after chronic morphine administration. Sci. Signal. 3, ra54-ra54, doi:10.1126/scisignal.2000807 (2010).
19. He, S.-Q. et al. Facilitation of μ-opioid receptor activity by preventing δ-opioid receptor-mediated codegradation. Neuron 69, 120-131, doi:https://doi.org/10.1016/j.neuron.2010.12.001 (2011).
20. Filizola, M., Olmea, O. & Weinstein, H. Prediction of heterodimerization interfaces of G-protein coupled receptors with a new subtractive correlated mutation method. Protein Eng. Des. Sel. 15, 881-885, doi:10.1093/protein/15.11.881 (2002).
21. Liu, X., Kai, M., Jin, L. & Wang, R. Computational study of the heterodimerization between μ and δ receptors. J. Comput. Aided Mol. Des. 23, 321-332, doi:10.1007/s10822-009-9262-7 (2009).
22. Yekkirala, A. S., Kalyuzhny, A. E. & Portoghese, P. S. Standard opioid agonists activate heteromeric opioid receptors: evidence for morphine and [d-Ala(2)-MePhe(4)-Glyol(5)]enkephalin as selective μ-δ agonists. ACS Chem. Neurosci. 1, 146-154, doi:10.1021/cn9000236 (2010).
23. Metcalf, M. D. et al. The δ opioid receptor agonist SNC80 selectively activates heteromeric μ-δ opioid receptors. ACS Chem. Neurosci. 3, 505-509, doi:10.1021/cn3000394 (2012).
24. Derouiche, L., Pierre, F., Doridot, S., Ory, S. & Massotte, D. Heteromerization of endogenous Mu and Delta opioid receptors induces ligand-selective co-targeting to Lysosomes. Molecules 25, 4493 (2020).
25. Gomes, I. et al. Identification of a μ-δ opioid receptor heteromer-biased agonist with antinociceptive activity. Proc. Natl. Acad. Sci. U.S.A. 110, 12072-12077, doi:10.1073/pnas. 1222044110 (2013).
26. Dijkman, P. M. et al. Dynamic tuneable G protein-coupled receptor monomer-dimer populations. Nat. Commun. 9, 1710, doi:10.1038/s41467-018-03727-6 (2018).
27. Johnston, J. M. et al. Making structural sense of dimerization interfaces of delta opioid receptor homodimers. Biochemistry 50, 1682-1690, doi:10.1021/bi101474v (2011).
28. Gomes, I. et al. Heterodimerization of mu and delta opioid receptors: A role in opiate synergy. J. Neurosci. 20, RC110-RC110, doi:10.1523/JNEUROSCI.20-22-j0007.2000 (2000).
29. Moller, J. et al. Single-molecule analysis reveals agonist-specific dimer formation of μ-opioid receptors. Nat. Chem. Biol. 16, 946-954, doi:10.1038/s41589-020-0566-1 (2020).

### REFERENCES (HOMODIMER BLOCKER SUPPLEMENTARY REFERENCES)

1. Conklin, B. R. et al. Substitution of three amino acids switches receptor specificity of Gqα to that of Giα. Nature 363, 274-276 (1993).
2. Yokoyama, T., Kato, N. & Yamada, N. Development of a high-throughput bioassay to screen melatonin receptor agonists using human melatonin receptor expressing CHO cells. Neurosci. Lett. 344, 45-48 (2003).
3. Han, Y. et al. Allosteric communication between protomers of dopamine class A GPCR dimers modulates activation. Nat. Chem. Biol. 5, 688-695 (2009).
4. Morise, J. et al. AMPA receptors in the synapse turnover by monomer diffusion. Nat. Commun. 10, 5245 (2019).
5. Tsunoyama, T. A. et al. Super-long single-molecule tracking reveals dynamic-anchorage-induced integrin function. Nat. Chem. Biol. 14, 497-506 (2018).
6. Kasai, R. S. et al. Full characterization of GPCR monomer-dimer dynamic equilibrium by single molecule imaging. J. Cell Biol. 192, 463-480 (2011).
7. Kasai, R. S. et al. The Class-A GPCR dopamine D2 receptor forms transient dimers stabilized by agonists: detection by single-molecule tracking. Cell Biochem. Biophys. 76, 29-37 (2018).
8. Kasai, R. S., Fujiwara, T. K. & Kusumi, A. Metastable GPCR dimers trigger the basal signal by recruiting G-proteins. bioRxiv, 2020.02.10.929588 (2020).
9. Andrei D. Polyanin & Nazaikinskii, V. E. Handbook of linear partial differential equations for engineers and scientists, (CRC press, Boca Raton, 2015).
10. NIST Digital Library of Mathematical Functions. https://dlmf.nist.gov

1-6. (canceled)

7. A method of prevention and/or treatment of opioid tolerance or opioid dependence comprising administering a peptide which inhibits the formation of heterodimers or homodimers formed from one or two types of opioid receptor selected from the group consisting of the μ-type (MOR), the κ-type (KOR) and the δ-type (DOR) to a subject.

8. The method of prevention and/or treatment of opioid tolerance or opioid dependence according to claim 7, wherein the peptide comprises any one amino acid sequence selected from the group consisting of the amino acid sequences of SEQ ID NOs: 1 to 40, amino acid sequences having one or more conservative amino acid substitutions in the amino acid sequences of SEQ ID NOs: 1 to 40, the amino acid sequences of SEQ ID NOs: 41 to 67, amino acid sequences having one or more conservative amino acid substitutions in the amino acid sequences of SEQ ID NOs: 41 to 67, and amino acid sequences having at least 80% amino acid sequence identity with any of these amino acid sequences.

9. The method of prevention and/or treatment of opioid tolerance or opioid dependence according to claim 7, wherein the peptide inhibits the formation of MOR and DOR heterodimers or KOR and DOR heterodimers.

10. The method of prevention and/or treatment of opioid tolerance or opioid dependence according to claim 9, wherein the peptide comprises any one amino acid sequence selected from the group consisting of the amino acid sequences of SEQ ID NOs: 1 to 30, amino acid sequences having one or more conservative amino acid substitutions in the amino acid sequence represented by SEQ ID NOs: 1 to 30, the amino acid sequences of SEQ ID NOs: 41 to 67, amino acid sequences having one or more conservative amino acid substitutions in the amino acid sequences of SEQ ID NOs: 41 to 67, and amino acid sequences having at least 80% amino acid sequence identity with any of these amino acid sequences.

11. The method of prevention and/or treatment of opioid tolerance or opioid dependence according to claim 7, wherein the peptide inhibits the formation of homodimers of MOR, KOR or DOR.

12. The method of prevention and/or treatment of opioid tolerance or opioid dependence according to claim 11, wherein the peptide comprises any one amino acid sequence selected from the group consisting of the amino acid sequences of SEQ ID NOs: 31 to 40, amino acid sequences having one or more conservative amino acid substitutions in the amino acid sequences of SEQ ID NOs: 31 to 40, and amino acid sequences having at least 80% amino acid sequence identity with any of these amino acid sequences.

13. A peptide comprising any one amino acid sequence selected from the group consisting of the amino acid sequences of SEQ ID NOs: 1 to 40, amino acid sequences having one or more conservative amino acid substitutions in the amino acid sequences of SEQ ID NOs: 1 to 40, the amino acid sequences of SEQ ID NOs: 41 to 67, amino acid sequences having one or more conservative amino acid substitutions in the amino acid sequences of SEQ ID NOs: 41 to 67, and amino acid sequences having at least 80% amino acid sequence identity with any of these amino acid sequences, wherein the peptide inhibits the formation of heterodimers or homodimers formed from one or two types of opioid receptor selected from the group consisting of the μ-type (MOR), κ-type (KOR), and δ-type (DOR).

14. The peptide according to claim 13 comprising any one amino acid sequence selected from the group consisting of the amino acid sequences of SEQ ID NOs: 1 to 30, amino acid sequences having one or more conservative amino acid substitutions in the amino acid sequences of SEQ ID NOs: 1 to 30, the amino acid sequences of SEQ ID NOs: 41 to 67, amino acid sequences having one or more conservative amino acid substitutions in the amino acid sequences of SEQ ID NOs: 41 to 67, and amino acid sequences having at least 80% amino acid sequence identity with any of these amino acid sequences, wherein the peptide inhibits the formation of MOR and DOR heterodimers, or KOR and DOR heterodimers.

15. The peptide according to claim 13 comprising any one amino acid sequence selected from the group consisting of the amino acid sequences of SEQ ID NOs: 31 to 40, amino acid sequences having one or more conservative amino acid substitutions in the amino acid sequences of SEQ ID NOs: 31 to 40, and amino acid sequences having at least 80% amino acid sequence identity with any of these amino acid sequences, wherein the peptide inhibits the formation of homodimers of MOR, KOR or DOR.

16. (canceled)

17. A method of enhancing opioid analgesia comprising administering a peptide which inhibits the formation of heterodimers or homodimers formed from one or two types of opioid receptor selected from the group consisting of the μ-type (MOR), the κ-type (KOR) and the δ-type (DOR) in combination with opioids to a subject who needs to use opioids, who uses opioids, or who plan to use opioids.
